SEQUENCE LISTING (82 sequences)

SEQ ID NO: 1 - protein, 22 aa, Homo sapiens
KMSPARPRTSCREACLQGWTEA

SEQ ID NO: 2 - protein, 24 aa, Homo sapiens
RRKMSPARPRTSCREACLQGWTEA

SEQ ID NO: 3 - protein, 421 aa, synthetic construct
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDNCRRMMRTKMRMRRMRRTRRKMRRKMSPARPRTSCREACLQGWTEADYKDDDDK

SEQ ID NO: 4 - protein, 408 aa, synthetic construct
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDELDYKDDDDK

SEQ ID NO: 5 - protein, 434 aa, synthetic construct
MRGSHHHHHHGMASMTGGQQMGRDLYDDDDKDRWGSELEAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 6 - protein, 407 aa, synthetic construct
MGSHHHHHHGSSGEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRSKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 7 - protein, 13 aa, synthetic construct
QSSQSVYNNNWLS

SEQ ID NO: 8 - protein, 7 aa, synthetic construct
DASKLES

SEQ ID NO: 9 - protein, 10 aa, synthetic construct
AGGFTGNVYT

SEQ ID NO: 10 - protein, 5 aa, synthetic construct
NNAIS

SEQ ID NO: 11 - protein, 10 aa, synthetic construct
GIDLSNNAIS

SEQ ID NO: 12 - protein, 9 aa, synthetic construct
VIGNTGDTY

SEQ ID NO: 13 - protein, 12 aa, synthetic construct
GPPSYSSSVKNI

SEQ ID NO: 14 - protein, 120 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSS

SEQ ID NO: 15 - protein, 450 aa, synthetic construct
EVQLLESGGGLVQPGGSLRLSCAVSGIDLSNNAISWVRQAPGKGLEYVGVIGNTGDTYYADSAKGRFTISRDSSKTTLYLQMNSLRAEDTAVYFCVRGPPSYSSSVKNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 16 - protein, 110 aa, synthetic construct
DIQMTQSPSSVSASVGDRVTITCQSSQSVYNNNWLSWLQQKPGKAPKRLIYDASKLESGVPSRFSGSGSGTDFTLTISSVQPEDAATYYCAGGFTGNVYTFGGGTKVEIK

SEQ ID NO: 17 - protein, 217 aa, synthetic construct
DIQMTQSPSSVSASVGDRVTITCQSSQSVYNNNWLSWLQQKPGKAPKRLIYDASKLESGVPSRFSGSGSGTDFTLTISSVQPEDAATYYCAGGFTGNVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 1416 bp, synthetic construct
  source 1..1416: mol_type = other DNA
atggccaggaagtccgctctgctcgctctggcacttctgcttctgggatttggacctgcttgggctgaggttcagctgctggaatctggcggaggattggttcagcctggcggctctctgagactgtcttgtgctgtgtctggcatcgacctgtccaacaacgccatctcctgggtccgacaggctcctggcaaaggcctggaatatgtgggcgtgatcggcaacaccggcgacacctactacgccgattctgccaagggcagattcaccatctctcgggactcctccaagaccacactgtacctgcagatgaactccctgagagccgaggataccgccgtgtacttttgtgtgcggggacctcctagctactcctccagcgtgaagaacatctggggccagggcacactggtcaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 19 - DNA, 717 bp, synthetic construct
  source 1..717: mol_type = other DNA
atggccaggaagtccgctctgctcgctctggcacttctgcttctgggatttggacctgcttgggctgacatccagatgacccagtctccatcctccgtgtctgcctctgtgggcgacagagtgaccatcacctgtcagtccagccaatccgtgtacaacaacaactggctgtcctggctgcagcagaagcctggcaaggctcccaagagactgatctacgacgcctccaagctggaatccggcgtgccctccagattttccggctctggctctggcaccgactttaccctgaccatctcctccgtgcagcctgaggatgccgccacctattattgtgctggaggcttcaccggcaacgtgtacacatttggcggaggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 20 - protein, 10 aa, synthetic construct
GFTFSRYNMN

SEQ ID NO: 21 - protein, 10 aa, synthetic construct
SISTSSNYIY

SEQ ID NO: 22 - protein, 7 aa, synthetic construct
GWGPFDY

SEQ ID NO: 23 - protein, 116 aa, synthetic construct
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGCGTLVTVSS

SEQ ID NO: 24 - protein, 11 aa, synthetic construct
RARQSIGTAIH

SEQ ID NO: 25 - protein, 7 aa, synthetic construct
YASESIS

SEQ ID NO: 26 - protein, 9 aa, synthetic construct
QQSGSWPYT

SEQ ID NO: 27 - protein, 107 aa, synthetic construct
DIQMTQSPSSLSASVGDRVTITCRARQSIGTAIHWYQQKPGCAPKLLIKYASESISGVPSRFSGSGSGTDFTLTISSLQPEDFATYYCQQSGSWPYTFGQGTKLEIK

SEQ ID NO: 28 - protein, 473 aa, synthetic construct
DIQMTQSPSSLSASVGDRVTITCRARQSIGTAIHWYQQKPGCAPKLLIKYASESISGVPSRFSGSGSGTDFTLTISSLQPEDFATYYCQQSGSWPYTFGQGTKLEIKGGGSGGSGGCPPCGGSGGEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1485 bp, synthetic construct
  source 1..1485: mol_type = other DNA
atggccaggaagtccgctctgctcgctctggcacttctgcttctgggatttggacctgcttgggctgacatccagatgacccagtctccatcctctctgtccgcctctgtgggcgacagagtgaccattacctgccgggccagacagtctatcggcaccgctatccactggtatcagcagaagcctggctgtgcccctaagctgctgattaagtacgcctccgagtccatctccggcgtgccctccagattttctggctctggatctggcaccgactttaccctgacaatctccagcctgcagcctgaggacttcgccacctactactgtcagcagtccggctcttggccttacacctttggtcagggcaccaagctggaaatcaagggcggaggttctggtggatctggcggatgtcctccttgtggtggaagcggcggagaggtgcagctggttgaatctggcggaggactggttaagcctggcggctctctgagactgtcttgtgctgcttctggcttcaccttcagccggtacaacatgaactgggtccgacaggctcctggcaaaggcctggaatgggtgtcctccatctccacctccagcaactacatctactacgccgactccgtgaagggcagattcaccttctccagagacaacgccaagaactccctggacctgcagatgtctggcctgagagctgaggacaccgctatctactactgcaccagaggctggggacccttcgattattggggctgtggaaccctggtcaccgtgtcatctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 30 - protein, 45 aa, Homo sapiens
TRRMMRTKMRMRRMRRTRRKMRRKMSPARPRTSCREACLQGWTEA

SEQ ID NO: 31 - protein, 46 aa, Homo sapiens
NCRRMMRTKMRMRRMRRTRRKMRRKMSPARPRTSCREACLQGWTEA

SEQ ID NO: 32 - protein, 47 aa, Homo sapiens
QRTRRMMRTKMRMRRMRRTRRKMRRKMSPARPRTSCREACLQGWTEA

SEQ ID NO: 33 - protein, 50 aa, Homo sapiens
RRRQRTRRMMRTKMRMRRMRRTRRKMRRKMSPARPRTSCREACLQGWTEA

SEQ ID NO: 34 - protein, 47 aa, Homo sapiens
GRTRRMMRTKMRMRRMRRTRRKMRRKMSPARPRTSCREACLQGWTEA

SEQ ID NO: 35 - protein, 49 aa, Homo sapiens
RRQRTRRMMRTKMRMRRMRRTRRKMRRKMSPARPRTSCREACLQGWTEA

SEQ ID NO: 36 - protein, 44 aa, Homo sapiens
RRMMRTKMRMRRMRRTRRKMRRKMSPARPRTSCREACLQGWTEA

SEQ ID NO: 37 - protein, 49 aa, Homo sapiens; identical to SEQ ID NO: 35

SEQ ID NO: 38 - protein, 48 aa, Homo sapiens
RQRTRRMMRTKMRMRRMRRTRRKMRRKMSPARPRTSCREACLQGWTEA

SEQ ID NO: 39 - protein, 46 aa, Homo sapiens
MCRRMMRTKMRMRRMRRTRRKMRRKMSPARPRTSCREACLQGWTEA

SEQ ID NO: 40 - protein, 50 aa, Homo sapiens
DQRQRTRRMMRTKMRMRRMRRTRRKMRRKMSPARPRTSCREACLQGWTEA

SEQ ID NO: 41 - protein, 51 aa, Homo sapiens
RRRRQRTRRMMRTKMRMRRMRRTRRKMRRKMSPARPRTSCREACLQGWTEA

SEQ ID NO: 42 - protein, 51 aa, Homo sapiens
QRRRQRTRRMMRTKMRMRRMRRTRRKMRRKMSPARPRTSCREACLQGWTEA

SEQ ID NO: 43 - protein, 50 aa, Homo sapiens; identical to SEQ ID NO: 33

SEQ ID NO: 44 - protein, 50 aa, Homo sapiens
RRRERTRRMMRTKMRMRRMRRTRRKMRRKMSPARPRTSCREACLQGWTEA

SEQ ID NO: 45 - protein, 50 aa, Homo sapiens
QRRQRTRRMMRTKMRMRRMRRTRRKMRRKMSPARPRTSCREACLQGWTEA

SEQ ID NO: 46 - protein, 49 aa, Homo sapiens
RRQWTRRMMRTKMRMRRMRRTRRKMRRKMSPARPRTSCREACLQGWTEA

SEQ ID NO: 47 - protein, 43 aa, Homo sapiens
RMMRTKMRMRRMRRTRRKMRRKMSPARPRTSCREACLQGWTEA

SEQ ID NO: 48 - protein, 48 aa, Homo sapiens; identical to SEQ ID NO: 38

SEQ ID NO: 49 - protein, 50 aa, Homo sapiens
GRRQRTRRMMRTKMRMRRMRRTRRKMRRKMSPARPRTSCREACLQGWTEA

SEQ ID NO: 50 - protein, 49 aa, Homo sapiens
AFKRTRRMMRTKMRMRRMRRTRRKMRRKMSPARPRTSCREACLQGWTEA

SEQ ID NO: 51 - protein, 53 aa, Homo sapiens
NAKRRRRQRTRRMMRTKMRMRRMRRTRRKMRRKMSPARPRTSCREACLQGWTE

SEQ ID NO: 52 - protein, 53 aa, Homo sapiens
CVRRRRQRTRRMMRTKMRMRRMRRTRRKMRRKMSPARPRTSCREACLQGWTEA

SEQ ID NO: 53 - protein, 49 aa, Homo sapiens; identical to SEQ ID NO: 35

SEQ ID NO: 54 - protein, 48 aa, Homo sapiens; identical to SEQ ID NO: 38

SEQ ID NO: 55 - protein, 53 aa, Homo sapiens; identical to SEQ ID NO: 51

SEQ ID NO: 56 - protein, 53 aa, Homo sapiens
CFAKRRRRQRTRRMMRTKMRMRRMRRTRRKMRRKMSPARPRTSCREACLQGWT

SEQ ID NO: 57 - protein, 44 aa, Homo sapiens; identical to SEQ ID NO: 36

SEQ ID NO: 58 - protein, 49 aa, Homo sapiens
PPLCLRRMMRTKMRMRRMRRTRRKMRRKMSPARPRTSCREACLQGWTEA

SEQ ID NO: 59 - protein, 48 aa, Homo sapiens
DHPCRRMMRTKMRMRRMRRTRRKMRRKMSPARPRTSCREACLQGWTEA

SEQ ID NO: 60 - protein, 47 aa, Homo sapiens
GNCRRMMRTKMRMRRMRRTRRKMRRKMSPARPRTSCREACLQGWTEA

SEQ ID NO: 61 - protein, 45 aa, Homo sapiens
CRRMMRTKMRMRRMRRTRRKMRRKMSPARPRTSCREACLQGWTEA

SEQ ID NO: 62 - protein, 45 aa, Homo sapiens; identical to SEQ ID NO: 61

SEQ ID NO: 63 - protein, 46 aa, Homo sapiens
TCRRMMRTKMRMRRMRRTRRKMRRKMSPARPRTSCREACLQGWTEA

SEQ ID NO: 64 - protein, 46 aa, Homo sapiens
ICRRMMRTKMRMRRMRRTRRKMRRKMSPARPRTSCREACLQGWTEA

SEQ ID NO: 65 - protein, 45 aa, Homo sapiens; identical to SEQ ID NO: 61

SEQ ID NO: 66 - protein, 8 aa, synthetic construct
QSVYNNNW

SEQ ID NO: 67 - protein, 16 aa, synthetic construct
VIGNTGDTYYADSAKG

SEQ ID NO: 68 - protein, 7 aa, synthetic construct
GIDLSNN

SEQ ID NO: 69 - protein, 5 aa, synthetic construct
GNTGD

SEQ ID NO: 70 - protein, 8 aa, synthetic construct
GIDLSNNA

SEQ ID NO: 71 - protein, 7 aa, synthetic construct
IGNTGDT

SEQ ID NO: 72 - protein, 14 aa, synthetic construct
VRGPPSYSSSVKNI

SEQ ID NO: 73 - protein, 5 aa, synthetic construct
RYNMN

SEQ ID NO: 74 - protein, 17 aa, synthetic construct
SISTSSNYIYYADSVKG

SEQ ID NO: 75 - protein, 7 aa, synthetic construct
GFTFSRY

SEQ ID NO: 76 - protein, 6 aa, synthetic construct
STSSNY

SEQ ID NO: 77 - protein, 8 aa, synthetic construct
GFTFSRYN

SEQ ID NO: 78 - protein, 8 aa, synthetic construct
ISTSSNYI

SEQ ID NO: 79 - protein, 9 aa, synthetic construct
TRGWGPFDY

SEQ ID NO: 80 - protein, 6 aa, synthetic construct
QSIGTA

SEQ ID NO: 81 - protein, 241 aa, synthetic construct
DIQMTQSPSSLSASVGDRVTITCRARQSIGTAIHWYQQKPGCAPKLLIKYASESISGVPSRFSGSGSGTDFTLTISSLQPEDFATYYCQQSGSWPYTFGQGTKLEIKGGGSGGSGGCPPCGGSGGEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGCGTLVTVSS

SEQ ID NO: 82 - protein, 22 aa, synthetic construct
KMSPARPRTSCREACLQGWTEA

(19) United States
(12) Patent Application Publication
(10) Pub. No.: US 2025/0376509 A1
(43) Pub. Date: Dec. 11, 2025

(54) METHODS FOR TREATMENT OF MYELOPROLIFERATIVE NEOPLASMS

(71) Applicant: Janssen Biotech, Inc., Horsham, PA (US)

(72) Inventors: Laura BARREYRO, Huntingdon Valley, PA (US); Anna KUCHNIO, Edegem (BE); Oliver LOMAS, Buckinghamshire (GB); Ulrike PHILIPPAR, Antwerpen (BE); Isha TANEJA, Lansdale, PA (US)

(21) Appl. No.: 18/955,404

(22) Filed: Nov. 21, 2024

Related U.S. Application Data
(60) Provisional application No. 63/601,323, filed on Nov. 21, 2023.
(60) Provisional application No. 63/648,951, filed on May 17, 2024.

(51) Int. Cl. C07K 16/18; A61K 39/00; C07K 16/28

(52) U.S. Cl. CPC C07K 16/18; C07K 16/2809; A61K 2039/505; C07K 2317/31; C07K 2317/92; C07K 2317/94

(57) ABSTRACT

Methods of inhibiting the growth or proliferation, or treating, myeloproliferative neoplasm using bi-specific molecules that bind to mutant calreticulin and CD3 are described.

### CROSS-REFERENCE TO RELATED APPLICATION

This application claims priority to each of U.S. Provisional Patent Application No. 63/601,323, filed Nov. 21, 2023, and U.S. Provisional Patent Application No. 63/648,951, filed May 17, 2024, the disclosure of each of the foregoing applications is incorporated herein by reference in its entirety.

### FIELD

The present application relates to bi-specific molecules that bind to mutant calreticulin and CD3 for use in methods of treating or reducing the severity of myeloproliferative neoplasms in a subject.

### REFERENCE TO SEQUENCE LISTING SUBMITTED ELECTRONICALLY

This application contains a sequence listing, which is submitted electronically. The information contained in the electronic sequence listing (name: JBI6861PCT1-Sequence-Listing.xml; size: 79.597 bytes; and date of creation: Mar. 12, 2025) is incorporated herein by reference in its entirety.

### BACKGROUND

Myeloproliferative Neoplasms (MPNs) are clonal disorders of hematopoiesis arising in the hematopoietic stem cell (HSC) compartment that are characterized by excessive production of mature blood cells of the myeloid lineage. Transformation to secondary acute myeloid leukemia (sAML) represents a significant cause of death among MPN patients. Current treatment options for MPN patients are limited to symptomatic treatment. At present, the only treatment in MPN that has the potential to cure the disease or prolong survival is stem cell transplantation (SCT). Transplant-related death or severe morbidity occurs in more than half of transplant recipients and therefore necessitates risk justification in the individual patient. Therefore, identification of novel therapeutic approaches with a disease-modifying effect for the treatment of MPNs and intercepting their progression to sAML is an unmet medical need.

Mutations in JAK2, thrombopoietin receptor (TPOR, also known as myeloproliferative leukemia protein or MPL), and calreticulin (CALR) are phenotypic drivers in the pathogenesis of MPN. CALR mutations (CALRmut) are the second most frequent in MPN. CALRmut are insertions or deletions resulting in a frameshift in the last exon of the gene, resulting in loss of the KDEL ER-retention motif and generation of a 36 amino acid positively charged C-terminal neoantigen. Due to loss of the KDEL motif, CALRmut is not confined to the ER. Specifically, a tetrameric complex formed between 2 CALRmut and 2 MPL proteins is shuttled through the Golgi apparatus and is presented on the cell membrane, leading to constitutive activation of downstream kinase JAK2. Cell surface presentation of CALRmut/MPL complex is required for constitutive activation of JAK2 and oncogenic transformation. In contrast to wild type cells, where MPL is only present on the cell surface with mature glycosylation, MPL in complex with CALRmut remains in an immature glycosylation status on the cell surface.

As CALRmut is expressed on the cell surface in MPNs, it can be considered as a target for immunotherapeutic treatment. There is a need for disease modifying treatments of MPN, which target CALRmut. Disclosed herein are bispecific antibodies, and antigen-binding fragments thereof, that bind to CALRmut and CD3 for use in treating or reducing the severity of myeloproliferative neoplasms in a subject.

### SUMMARY

Provided herein is a method of inhibiting the growth or proliferation of a myeloproliferative neoplasm (MPN) or treating the MPN, the method comprises administering to a subject, such as a human subject, in need thereof a treatment dose of 0.6-400 mg per administration of an anti-mutant calreticulin (CALRmut)/anti-CD3 bispecific antibody, wherein the anti-CALRmut/anti-CD3 bispecific antibody comprises a first antigen binding domain that binds specifically to CALRmut, and a second antigen binding domain that binds specifically to CD3ε.

In certain embodiments, the first antigen binding domain comprises a first HCDR1, a first HCDR2 and a first HCDR3 of a first heavy chain variable region (VH1) of SEQ ID NO: 14, and wherein the first antigen binding domain comprises a first light chain complementarity determining region (LCDR) 1, a first LCDR2, and a first LCDR3 of a first light chain variable region (VL1) of SEQ ID NO: 16. In certain embodiments, the second antigen binding domain comprises a second HCDR1, a second HCDR2, and a second HCDR3 of a second heavy chain variable region (VH2) of SEQ ID NO:23 and a second LCDR1, a second LCDR2, and a second LCDR3 of second light chain variable region (VL2) of SEQ ID NO:27.

In certain embodiments, the anti-CALRmut/anti-CD3 bispecific antibody useful for the invention comprises a heavy chain, a light chain, and a stapled single chain fragment variable (spFv) chain,
wherein the heavy chain associates with the light chain to form an antigen binding site that immunospecifically binds to a mutant calreticulin (CALRmut);
wherein the spFv chain immunospecifically binds to CD3; and wherein
a) the heavy chain comprises a heavy chain complementarity determining region (CDR) 1 (CDR1) comprising the amino acid sequence of SEQ ID NO: 11, a heavy chain CDR2 comprising the amino acid sequence of SEQ ID NO: 12, and a heavy chain CDR3 comprising the amino acid sequence of SEQ ID NO: 13;
the light chain comprises a light chain CDR1 comprising the amino acid sequence of SEQ ID NO: 7, a light chain CDR2 comprising the amino acid sequence of SEQ ID NO: 8, and a light chain CDR3 comprising the amino acid sequence of SEQ ID NO: 9; and
the spFv chain comprises a light chain CDR1 comprising the amino acid sequence of SEQ ID NO: 24, a light chain CDR2 comprising the amino acid sequence of SEQ ID NO: 25, a light chain CDR3 comprising the amino acid sequence of SEQ ID NO: 26, a heavy chain CDR1 comprising the amino acid sequence of SEQ ID NO: 20, a heavy chain CDR2 comprising the amino acid sequence of SEQ ID NO: 21, and a heavy chain CDR3 comprising the amino acid sequence of SEQ ID NO: 22;
b) the heavy chain comprises a heavy chain CDR1 comprising the amino acid sequence of SEQ ID NO: 68, a heavy chain CDR2 comprising the amino acid sequence of SEQ ID NO: 69, and a heavy chain CDR3 comprising the amino acid sequence of SEQ ID NO: 13;
the light chain comprises a light chain CDR1 comprising the amino acid sequence of SEQ ID NO: 7, a light chain CDR2 comprising the amino acid sequence of SEQ ID NO: 8, and a light chain CDR3 comprising the amino acid sequence of SEQ ID NO: 9; and
the spFv chain comprises a light chain CDR1 comprising the amino acid sequence of SEQ ID NO: 24, a light chain CDR2 comprising the amino acid sequence of SEQ ID NO: 25, a light chain CDR3 comprising the amino acid sequence of SEQ ID NO: 26, a heavy chain CDR1 comprising the amino acid sequence of SEQ ID NO: 75, a heavy chain CDR2 comprising the amino acid sequence of SEQ ID NO: 76, and a heavy chain CDR3 comprising the amino acid sequence of SEQ ID NO: 22;
c) the heavy chain comprises a heavy chain CDR1 comprising the amino acid sequence of SEQ ID NO: 10, a heavy chain CDR2 comprising the amino acid sequence of SEQ ID NO: 67, and a heavy chain CDR3 comprising the amino acid sequence of SEQ ID NO: 13;
the light chain comprises a light chain CDR1 comprising the amino acid sequence of SEQ ID NO: 7, a light chain CDR2 comprising the amino acid sequence of SEQ ID NO: 8, and a light chain CDR3 comprising the amino acid sequence of SEQ ID NO: 9; and
the spFv chain comprises a light chain CDR1 comprising the amino acid sequence of SEQ ID NO: 24, a light chain CDR2 comprising the amino acid sequence of SEQ ID NO: 25, a light chain CDR3 comprising the amino acid sequence of SEQ ID NO: 26, a heavy chain CDR1 comprising the amino acid sequence of SEQ ID NO: 73, a heavy chain CDR2 comprising the amino acid sequence of SEQ ID NO: 74, and a heavy chain CDR3 comprising the amino acid sequence of SEQ ID NO: 22; or
d) the heavy chain comprises a heavy chain CDR1 comprising the amino acid sequence of SEQ ID NO: 70, a heavy chain CDR2 comprising the amino acid sequence of SEQ ID NO: 71, and a heavy chain CDR3 comprising the amino acid sequence of SEQ ID NO: 72;
the light chain comprises a light chain CDR1 comprising the amino acid sequence of SEQ ID NO: 66, a light chain CDR2 comprising the amino acid sequence of DAS, and a light chain CDR3 comprising the amino acid sequence of SEQ ID NO: 9; and
the spFv chain comprises a light chain CDR1 comprising the amino acid sequence of SEQ ID NO: 81, a light chain CDR2 comprising the amino acid sequence of YAS, a light chain CDR3 comprising the amino acid sequence of SEQ ID NO: 26, a heavy chain CDR1 comprising the amino acid sequence of SEQ ID NO: 78, a heavy chain CDR2 comprising the amino acid sequence of SEQ ID NO: 79, and a heavy chain CDR3 comprising the amino acid sequence of SEQ ID NO: 80.

In certain embodiments, the heavy chain comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 14. In preferred embodiments, the VH comprises the amino acid sequence of SEQ ID NO: 14.

In certain embodiments, the light chain comprises a light chain variable region (VL) comprising an amino acid sequence that is at least 90%, 91%, 92%, 93%, 94%, 95%, 96%, 97%, 98%, or 99% identical to the amino acid sequence of SEQ ID NO: 16. In preferred embodiments, the VL comprises the amino acid sequence of SEQ ID NO: 16.

In certain embodiments, the heavy chain variable region (VH) comprises the amino acid sequence of SEQ ID NO: 14, and the light chain variable region (VL) comprises the amino acid sequence of SEQ ID NO: 16.

In certain embodiments, the bispecific antibody is an IgG. In certain embodiments, the bispecific antibody comprises an IgG1 isotype Fc region. In certain embodiments, the bispecific antibody further comprises L234A, L235A, and D265S substitutions in the Fc region. In the certain embodiments, the bispecific antibody further comprises knob-into-hole (KiH) substitutions. In certain embodiments, the bispecific antibody further comprises H435R and Y436F substitutions in the Fc region.

In certain embodiments, the heavy chain comprises the amino acid sequence of SEQ ID NO: 15. In certain embodiments, the light chain comprises the amino acid sequence of SEQ ID NO: 17. In certain embodiments, the spFv chain comprises the amino acid sequence of SEQ ID NO: 28.

In certain embodiments, the subject is administered a treatment dose of at least 0.6 mg, such as 0.6-400 mg, 5-100 mg, 20-200 mg of the bispecific antibody or bispecific antigen-binding fragment thereof per administration.

In certain embodiment, the method further comprises administering to the subject one or more step-up doses of the anti-CALRmut/anti-CD3 bispecific antibody prior to the administration of the treatment dose, wherein none of the step-up doses exceeds the treatment dose. In certain embodiment, a step-up dose, e.g., 0.6 mg, 1 mg, or 1.2 mg per administration, of the anti-CALRmut/anti-CD3 bispecific antibody is administered to the subject 3-8 days, such as 3, 4, 5, 6, 7, or 8 days, prior to the initial administration of the treatment dose, e.g., to mitigate injection site reactions and CRS.

In certain embodiments, the bispecific antibody or bispecific antigen-binding fragment thereof is administered once every 1, 2, 3, 4, 5, 6, 7 or 8 weeks, preferably the bispecific antibody or bispecific antigen-binding fragment thereof is administered once every 3 weeks.

In certain embodiments, the application relates to a method of treating a MPN, the method comprising subcutaneously administering to a human subject in need thereof, once every three weeks, a treatment dose of 1.2-400 mg, such as 5-100 mg or 20-200 mg, per administration of an anti-CALRmut/anti-CD3 bispecific antibody, wherein the anti-CALRmut/anti-CD3 bispecific antibody comprises a first heavy chain having the amino acid sequence of SEQ ID NO: 15, a first light chain having the amino acid sequence of SEQ ID NO: 17 and a second heavy chain having the amino acid sequence of SEQ ID NO:28, optionally, the method further comprises subcutaneously administering to the subject a step-up dose of 0.6 or 1.2 mg per administration of the anti-CALRmut/anti-CD3 bispecific antibody one week before the initial administration of the treatment dose.

In certain embodiments, the MPN is characterized by the presence of a mutant calreticulin (CALR). In certain embodiments, the subject in need of the treatment has one or more CALR mutations that are Type 1, Type 1-like, Type 2, or Type 2-like. Other types of mutation patterns are also envisioned in some aspects of the invention, such as those mutation patterns described by, e.g., Pietra et al. Leukemia. 2016 February; 30(2):431.

In certain embodiments, the subject has a splenectomy. In other embodiments, the subject has an allograft, e.g., an allogeneic bone marrow or stem cells transplant.

In certain embodiments, the subject is ineligible, intolerant or resistant to JAK inhibitor therapy.

In certain embodiments, the subject has been administered a prior therapy. For example, the subject can be treated with one or more lines of other treatments, such as a treatment with a JAK inhibitor and/or hydroxyurea. Optionally, the subject has failed one or more lines of prior treatments.

In certain embodiments, the MPN is selected from the group consisting of chronic myelogenous leukemia, polycythemia vera, primary myelofibrosis (MF), essential thrombocythemia (ET), chronic neutrophilic leukemia, and chronic eosinophilic leukemia. In certain embodiments, the subject has a myelodysplastic syndrome selected from ET, prefibrotic MF, overt primary MF, and accelerated blast phase MF.

In certain embodiments, the subject is diagnosed with ET, particularly an ET with high-risk of thrombosis or hemorrhage and intolerant or resistant or refractory to hydroxyurea. Preferably, the method results in at least one of: 1) normal spleen size on imaging; and 2) platelet count ≤400×10⁹/L and/or white cell count ≤10×10⁹/L in peripheral blood.

In certain embodiments, the subject is diagnosed with MF. For example, the subject can have a primary MF, such as a primary MF with a Dynamic International Prognostic Scoring System (DIPSS, Passamonti 2010, Blood. 115:1703-1708) risk score of Intermediate 1 (Int-1), Intermediate 2 (Int-2) or High-Risk (HR), optionally with a blast percentage not consistently exceeding 20% in blood or bone marrow. In certain embodiments, the subject has post-ET MF, such as a post-ET MF with a Myelofibrosis Secondary to PV and ET-Prognostic Model (MYSEC-PM, Passamonti 2017, Leukemia. 31:2726-2731) risk score of Int-1, Int-2 or HR, optionally with a blast percentage not consistently exceeding 20% in blood or bone marrow. Preferably, a method of the application results in a reduction in splenic volume of the subject in need of treating MF compared to a baseline splenic volume measured before administration of the anti-CALRmut/anti-CD3 bispecific antibody, preferably the splenic volume is reduced by at least 35% as compared to a baseline splenic volume measured before administration of the anti-CALRmut/anti-CD3 bispecific antibody.

In certain embodiments, the MPN is MF and the method results in at least one of: 1) bone marrow age-adjusted normocellularity, 2) <5% blasts, and 3) Hemoglobin ≥10 g/dL, neutrophil count ≥1×10⁹/L, and/or platelet count ≥100×10⁹/L in peripheral blood. In certain embodiments, the method modified the MF disease, e.g., it exerts a clinically meaningful impact on survival outcomes and/or restoration of normal hematopoiesis in the subject in conjunction with improvement in bone marrow fibrosis through a substantial and durable reduction in the clonal burden of disease. See, e.g., Pemmaraju et al 2022, J Clin Oncol. 40(26):3032-3036, for disease modification in MF, the relevant content of which is incorporated herein by reference in its entirety.

In certain embodiments, the method decreases soluble CALRmut levels in serum of the subject, preferably the soluble CALRmut levels are reduced by at least 50%, compared to a baseline level measured before administration of the anti-CALRmut/anti-CD3 bispecific antibody.

In another embodiment, the method results in a reduction of CALRmut positive cells in bone marrow samples.

In other embodiments, the method results in improved bone marrow architecture, such as reduction in bone marrow reticulin fibrosis and bone marrow cellularity.

In certain embodiments, a method of the application further comprises administering to the subject at least one first additional therapeutic prior to being administered with the treatment dose of the anti-CALRmut/anti-CD3 bispecific antibody, or prior to being administered with the set-up dose of the anti-CALRmut/anti-CD3 bispecific antibody. For example, at least one first additional therapeutic can be a glucocorticosteroid, antihistamine, antipyretic, antiemetic, or any combination thereof.

In certain embodiments, a method of the application further comprises administering to the subject at least one second additional therapeutic prior to, and optionally after, being administered with the treatment dose of the anti-CALRmut/anti-CD3 bispecific antibody. For example, the at least one second additional therapeutic can be an H1 antagonist, H2 antagonist or a Leukotriene inhibitor, or any combination thereof.

The application also relates to the anti-CALRmut/anti-CD3 bispecific antibody for use in the method of treating MPN of any of the foregoing embodiments of the application.

Further aspects, features and advantages of the present invention will be better appreciated upon a reading of the following detailed description of the invention and claims.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1 depicts an anti-CALRmut x CD3 bispecific antibody, wherein AAS (L234A, L235A, D265S); CALRmut, mutant calreticulin; CD, cluster of differentiation; Fab, fragment antigen-binding; Fc, fragment crystallizable; RF, H435R, Y436F; spFv, stapled single chain fragment variable.

FIG. 2 depicts the antitumor efficacy of an anti-CALRmut/anti-CD3 bispecific antibody (C3CRB73) in SC ELF-153 MPL CALRdel52 xenografts in T cell-humanized NSG mice. T cell-humanized (T cell injection indicated by first arrow) NSG mice injected SC with ELF-153 MPL CALRdel52 tumors were dosed IP with 1, 5, or 20 mg/kg C3CRB73 (dosing indicated by second to last arrows). Tumor volume was measured twice weekly, and results are presented as the mean tumor volume SEM for each group. Tumor volume curves are shown only when two thirds of animals in each group were still in study.

FIGS. 3A and 3B depict the antitumor efficacy of C3CRB73 in disseminated OCI-M2 MPL CALRins5 xenografts in T cell-humanized NSG mice. FIG. 3A depicts animal body weight loss data and FIG. 3B depicts survival data. T cell-humanized (T cell injection indicated by first arrow) NSG mice injected IV with OCI-M2 MPL CALRins5 tumors were dosed IP with indicated doses and antibodies (dosing indicated by second to last arrows). Negative clinical signs and/or ≥20% body weight loss were used as a surrogate endpoint for death. Body weight curves are shown only when two thirds of animals in each group were still in study.

FIGS. 4A and 4B depict the mutational pattern of CALR mutations in MPN patients. The wide black bar represents exon 9 of CALR, the narrow bar the 3′ UTR of the gene, the thin line intronic and intergenic regions. Indicated in FIG. 4A are the cDNA sequences in the beginning and end of exon 9. Below the cDNA sequences are the amino acid sequences derived from the three alternative reading frames. FIG. 4B shows that the three reading frames result in different peptide compositions, especially with respect to the charge of amino acids. FIG. 4C illustrates the spectrum of CALRmut myeloproliferative disease defined by CALRmut, degree of fibrosis and blood counts and their treatments.

FIG. 5 depicts serum concentration-time profile following a single 0.5 mg/kg IV dose of C3CRB73 in male cynomolgus monkeys (n=3/group).

FIGS. 6A-6F show that T-cell activation mediated by C3CRB73 only occurred in the presence of CALRmut-positive tumor cells. FIGS. 6A and 6B show that no cytotoxicity was observed in healthy hematopoietic cells. FIGS. 6C-6F show that C3CRB73 mediated potent T-cell cytotoxicity to CALRmut patient-derived CD34+ cells ex vivo.

FIG. 7 depicts how to incorporate a step-up dosing schedule in the Schedule of Activities if 1 or more step-up doses are administered.

FIG. 8 depicts a schematic overview of the study design.

FIG. 9 depicts cohort results on the recovery of hemoglobin following treatment with COMPOUND 1.

### DETAILED DESCRIPTION

The present disclosure provides embodiments for bispecific antibodies that bind to CALRmut and CD3 for use in treating or reducing the severity of myeloproliferative neoplasms (MPN) in a subject.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +/−10% and remain within the scope of the disclosed embodiments. Additionally, although a value may be preceded by the term “about” the exact value is also provided for herein, i.e., without the term “about”.

“Antigen” refers to any molecule (e.g., protein, peptide, polysaccharide, glycoprotein, glycolipid, nucleic acid, portions thereof, or combinations thereof) capable of being bound by an antigen binding domain or a T-cell receptor that is capable of mediating an immune response. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, and killed or inactivated whole cells or lysates.

“Antigen binding fragment” or “antigen binding domain” refers to a portion of the protein that binds an antigen. Antigen binding fragments may be synthetic, enzymatically obtainable or genetically engineered polypeptides and include portions of an immunoglobulin that bind an antigen, such as VH, the VL, the VH and the VL, Fab, Fab′, F(ab′)2, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv), stapled single chain Fv (spFv), or diabody. In some embodiments, an antibody fragment includes stapled single chain Fv (or spFv). Antigen binding fragments may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each HC is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes: IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

The term “antibody” molecule also encompasses whole or antigen binding fragments of domain, or single domain, antibodies, which can also be referred to as “sdAb” or “VHH”. Domain antibodies comprise either VH or VL that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference in its entirety). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO 2004/041862 and WO 2016/065323, each of which is hereby incorporated by reference in its entirety.

As used herein, the term “bispecific antibody” refers to an antibody that binds no more than two epitopes or two antigens. A bispecific antibody is characterized by a first variable heavy and light chain pair which has binding specificity for a first epitope (e.g., an epitope on a CALRmut antigen) and a second variable heavy and light chain pair that has binding specificity for a second epitope (e.g., an epitope on a T cell (e.g., CD3). As used herein, a “bispecific antibody” encompasses a bispecific antibody comprising one or more immunoglobulin (Ig) constant regions, as well as one or more bispecific antigen-binding fragments thereof.

“Immunospecifically” when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a Kd of less than about 1×10-8 M, as measured by a surface plasmon resonance assay or a cell binding assay. Phrases such as “anti-[antigen] antibody” (e.g., anti-CALRmut antibody) are meant to convey that the recited antibody specifically binds the recited antigen.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another, or another domain or peptide sequence can be between the two domains or sequences and such sequences would still be considered fused or linked to one another. In some embodiments, the various domains or proteins provided for herein are linked or fused directly to one another or a linker sequence(s), such as a glycine/serine, glycine/alanine linker or other types of peptide linkers generally known to link the two domains together. Two peptide sequences are linked directly if they are directly connected to one another or indirectly if there is a linker or other structure that links the two regions. A linker can be directly linked to two different peptide sequences or domains.

As used herein, the terms “variable region” and “variable domain” refer to the portions of the light and heavy chains of an antibody that include amino acid sequences of complementary determining regions (CDRs, e.g., CDR L1, CDR L2, CDR L3, CDR H1, CDR H2, and CDR H3) and framework regions (FRs). According to the methods used in this disclosure, the amino acid positions assigned to CDRs and FRs are defined according to Kabat (Sequences of Proteins of Immunological Interest, 5th Ed. Public Health Service, National Institutes of Health, Bethesda, MD. (1991)). Using this numbering system, the actual linear amino acid sequence may contain fewer or additional amino acids corresponding to a shortening of, or insertion into, a CDR (defined further herein) or FR (defined further herein) of the variable region. For example, a heavy chain variable region may include a single inserted residue (i.e., residue 52a according to Kabat) after residue 52 of CDR H2 and inserted residues (i.e., residues 82a, 82b, 82c, etc. according to Kabat) after residue 82 of heavy chain FR. The Kabat numbering of residues may be determined for a given antibody by alignment at regions of homology of the sequence of the antibody with a “standard” Kabat numbered sequence.

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton J Bmol Biol 263: 800-15, 1996). The correspondence between the various delineations and variable region numbering is described (see e.g., Lefranc et al. (2003) Dev Comp Immunol 27: 55-77; Honegger and Pluckthun, J Mol Biol (2001) 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Encode” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Fab” or “Fab fragment” refers to an antibody fragment composed of VH, CH1, VL and CL domains.

“Fv” or “Fv fragment” refers to an antibody fragment composed of the VH and the VL domains from a single arm of the antibody.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“Stapled single chain Fv” or “spFv”, described in WO2021030657A1, refer to a scFv that comprises one or more disulfide bonds between the VH and the linker or the VL and the linker. Typically, the spFv may comprise one disulfide bond between the VH and the linker, one disulfide bond between the VL and the linker, or two disulfide bonds between the VH and the linker and the VL and the linker. scFv molecules which comprise disulfide bonds between the VH and the VL are excluded from the term “spFv”.

In some embodiments, antibodies described herein may comprise mutations (e.g., amino acid substitutions, additions, and/or deletions) outside of the CDRs (i.e., in framework regions (FRs)). An amino acid substitution, addition, and/or deletion can be a substitution, addition, and/or deletion of one or more amino acids (e.g., 1, 2, 3, 4, 5, 6, 7, 8, or more). An amino acid substitution, addition, and/or deletion can be a substitution, addition, and/or deletion of eight or fewer, seven or fewer, six or fewer, five or fewer, four or fewer, three or fewer, or two or fewer single amino acids. In some embodiments, antibodies described herein may include amino acid substitutions, additions, and/or deletions in the constant regions (e.g., Fc region) of the antibody that, e.g., lead to decreased effector function, e.g., decreased complement-dependent cytolysis (CDC) antibody-dependent cell-mediated cytolysis (ADCC), antibody-dependent cell-mediated phagocytosis (ADCP), and/or decreased B-cell killing. For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcγRs or the complement factors. The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Mutations can be introduced in IgG1, IgG2 and IgG4 to reduce or silence Fc functionalities. Silencing mutations can include, but are not limited to IgG1 AA (F234A, L235A), IgG4 PAA (S228P, F234A, L235A), IgG2 AA (V234A, G237A), IgG1 FEA (L234F, L235E, D265A), or IgG1 FES (L234F/L235E/P33IS). In some embodiments, the disclosed antibody or antigen-binding fragment thereof can contain the IgG1 AA (F234A, L235A) mutation. In some embodiments, the disclosed antibody or antigen-binding fragment thereof can contain the IgG4 PAA (S228P, F234A, L235A) mutation. In some embodiments, the disclosed antibody or antigen-binding fragment thereof can contain the IgG2 AA (V234A, G237A) mutation. In some embodiments, the disclosed antibody or antigen-binding fragment thereof can contain the IgG1 FEA (L234F, L235E, D265A) mutation. In some embodiments, the disclosed antibody or antigen-binding fragment thereof can contain the IgG1 FES (L234F/L235E/P33IS) mutation. In some embodiments, the disclosed antibody or antigen-binding fragment thereof can contain the IgG1 L234A, L235A, and/or F405L mutations. In some embodiments, the disclosed antibody or antigen-binding fragment thereof can contain the S228P, L234A, L235A, F405L, and/or R409K mutations. In some embodiments, the disclosed antibody or antigen-binding fragment thereof can contain the IgG-AA Fc-L234A, L235A, and F405L.

The disclosed antibodies or antigen-binding fragments thereof can comprise an Fc region with one or more of the following properties: (a) reduced effector function when compared to the parent Fc; (b) reduced affinity to Fcγ RI, Fcγ RIIa, Fcγ RIIb, Fcγ RIIIb and/or Fcγ RIIIa; (c) reduced affinity to FcγRI; (d) reduced affinity to FcγRIIa; I reduced affinity to FcγRIIb; (f) reduced affinity to Fcγ RIIIb; or (g) reduced affinity to FcγRIIIa.

The term “CD3” refers to the human CD3 protein multi-subunit complex. The CD3 protein multi-subunit complex is composed to 6 distinctive polypeptide chains. These include a CD3γ chain (SwissProt P09693), a CD36 chain (SwissProt P04234), two CD3ε chains (SwissProt P07766), and one CD3 ζ chain homodimer (SwissProt 20963), and which is associated with the T cell receptor α and β chain. The term “CD3” includes any CD3 variant, isoform and species homolog which is naturally expressed by cells (including T cells) or can be expressed on cells transfected with genes or cDNA encoding those polypeptides, unless noted.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Also included are “NKT cells”, which refer to a specialized population of T cells that express a semi-invariant ap T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation and were found to be an important source of IL-17 and to induce robust CD8+ cytotoxic T cell response. Also included are “regulatory T cells” or “Tregs” which refer to T cells that suppress an abnormal or excessive immune response and play a role in immune tolerance. Tregs are typically transcription factor Foxp3-positive CD4+ T cells and can also include transcription factor Foxp3-negative regulatory T cells that are IL-10-producing CD4+ T cells.

The terms “mutant calreticulin” or “CALRmut” refer to mutant calreticulin identified and found to be associated with myeloid malignancies; see, Klampfl et al. (N Engl J Med 2013; 369:2379-2390 Dec. 19, 2013) and Nangalia et al. (N Engl J Med 2013; 369:2391-2405; EP 14 18 4835.8; PCT/EP2014/069638 and U.S. application Ser. No. 14/486,973). The terms “CALR” or “CALRwt” may be used interchangeably and refers to wild type calreticulin.

As used herein, the term “percent (%) identity” refers to the percentage of amino acid (or nucleic acid) residues of a candidate sequence, e.g., an antibody, or antigen-binding fragment thereof, of the present disclosure, that are identical to the amino acid (or nucleic acid) residues of a reference sequence, e.g., a wild-type antibody, or antigen-binding fragment thereof, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

$$100 \times ( fraction of A / B )$$

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In some embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position. A position may be altered by a substitution, deletion, or insertion. A substitution, deletion, or insertion may comprise a certain number of amino acids (e.g., 1, 2, 3, 4, 5, 6, 7, 8, 9, 10, 11, 12, 13, 14, 15, or more). When describing a substitution, deletion, or insertion of no more than n amino acids, this is meant that the substitution, deletion, or insertion comprises, e.g., 1, 2, 3, 4, 5, 6, 7, 8, 9, 10, 11, 12, 13, 14, 15, or n amino acids. The number or substitutions, deletions, or insertions can comprise a percent of the total sequence (e.g., 1%, 5%, 10%, 15%, 20%, or more) where the number of substitutions, deletions, or insertions alters 5%, 10%, 15%, 20% or more, of the amino acids in the total sequence.

“Polynucleotide” or “nucleic acid” refers to a synthetic molecule comprising a chain of nucleotides covalently linked by a sugar-phosphate backbone or other equivalent covalent chemistry. cDNA is a typical example of a polynucleotide. Polynucleotide may be a DNA or a RNA molecule.

“Vector” refers to an expression vector that can be utilized in a biological system or in a reconstituted biological system to direct the translation of a polypeptide encoded by a polynucleotide sequence present in the expression vector. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “individual,” “subject,” or “patient,” can be used interchangeably, means any animal, including mammals, such as mice, rats, other rodents, rabbits, dogs, cats, swine, cattle, sheep, horses, or primates, such as humans. As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the phrase “in need thereof” means that the subject has been identified as having a need for the method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from X to Y” means 1, 2, 3, 4, or 5.

### Anti-CALRmut x CD3 Bispecific Antibodies

In some embodiments, the bispecific antibody comprises one binding arm that binds to CD3ε and another binding arm that binds to CALRmut. In some embodiments, the CD3ε binding arm comprises a stapled single chain fragment variable (spFv) chain. In some embodiments, the CD3ε binding arm is described in WO 2021/240388, which is incorporated by reference in its entirety. In some embodiments, the CALRmut binding arm is any binding section disclosed herein.

In some embodiments, the anti-mutant calreticulin (CALRmut)/anti-CD3 bispecific antibody comprises a first antigen binding domain that binds specifically to CALRmut, and a second antigen binding domain that binds specifically to CD3R, wherein the first antigen binding domain comprises a first HCDR1, a first HCDR2 and a first HCDR3 of a first heavy chain variable region (VH1) of SEQ ID NO: 14, and wherein the first antigen binding domain comprises a first light chain complementarity determining region (LCDR) 1, a first LCDR2, and a first LCDR3 of a first light chain variable region (VL1) of SEQ ID NO: 16. In certain embodiments, the second antigen binding domain comprises a second HCDR1, a second HCDR2, and a second HCDR3 of a second heavy chain variable region (VH2) of SEQ ID NO:23 and a second LCDR1, a second LCDR2, and a second LCDR3 of second light chain variable region (VL2) of SEQ ID NO:27.

In some embodiments, the bispecific antibody comprises a heavy chain, a light chain, and a spFv chain,
wherein the heavy chain comprises a heavy chain CDR1 comprising the amino acid sequence of SEQ ID NO: 11, a heavy chain CDR2 comprising the amino acid sequence of SEQ ID NO: 12, and a heavy chain CDR3 comprising the amino acid sequence of SEQ ID NO: 13;
wherein the light chain comprises a light chain CDR1 comprising the amino acid sequence of SEQ ID NO: 7, a light chain CDR2 comprising the amino acid sequence of SEQ ID NO: 8, and a light chain CDR3 comprising the amino acid sequence of SEQ ID NO: 9;
wherein the spFv chain comprises a light chain CDR1 comprising the amino acid sequence of RARQSIGTAIH (SEQ ID NO: 24), a light chain CDR2 comprising the amino acid sequence of YASESIS (SEQ ID NO: 25), a light chain CDR3 comprising the amino acid sequence of QQSGSWPYT (SEQ ID NO: 26), a heavy chain CDR1 comprising the amino acid sequence of GFTFSRYNMN (SEQ ID NO: 20), a heavy chain CDR2 comprising the amino acid sequence of SISTSSNYIY (SEQ ID NO: 21), and a heavy chain CDR3 comprising the amino acid sequence of GWGPFDY (SEQ ID NO: 22);
wherein the heavy chain associates with the light to form an antigen binding site that immunospecifically binds to a mutant calreticulin (CALRmut); and
wherein the spFv chain immunospecifically binds to CD3.

In some embodiments, the bispecific antibody comprises a heavy chain, a light chain, and a spFv chain,
wherein the heavy chain comprises a heavy chain CDR1 comprising the amino acid sequence of SEQ ID NO: 68, a heavy chain CDR2 comprising the amino acid sequence of SEQ ID NO: 69, and a heavy chain CDR3 comprising the amino acid sequence of SEQ ID NO: 13;
wherein the light chain comprises a light chain CDR1 comprising the amino acid sequence of SEQ ID NO: 7, a light chain CDR2 comprising the amino acid sequence of SEQ ID NO: 8, and a light chain CDR3 comprising the amino acid sequence of SEQ ID NO: 9;
wherein the spFv chain comprises a light chain CDR1 comprising the amino acid sequence of RARQSIGTAIH (SEQ ID NO: 24), a light chain CDR2 comprising the amino acid sequence of YASESIS (SEQ ID NO: 25), a light chain CDR3 comprising the amino acid sequence of QQSGSWPYT (SEQ ID NO: 26), a heavy chain CDR1 comprising the amino acid sequence of GFTFSRY (SEQ ID NO: 75), a heavy chain CDR2 comprising the amino acid sequence of STSSNY (SEQ ID NO: 76), and a heavy chain CDR3 comprising the amino acid sequence of GWGPFDY (SEQ ID NO: 22);
wherein the heavy chain associates with the light to form an antigen binding site that immunospecifically binds to a mutant calreticulin (CALRmut); and
wherein the spFv chain immunospecifically binds to CD3.

In some embodiments, the bispecific antibody comprises a heavy chain, a light chain, and a spFv chain,
wherein the heavy chain comprises a heavy chain CDR1 comprising the amino acid sequence of SEQ ID NO: 10, a heavy chain CDR2 comprising the amino acid sequence of SEQ ID NO: 67, and a heavy chain CDR3 comprising the amino acid sequence of SEQ ID NO: 13;
wherein the light chain comprises a light chain CDR1 comprising the amino acid sequence of SEQ ID NO: 7, a light chain CDR2 comprising the amino acid sequence of SEQ ID NO: 8, and a light chain CDR3 comprising the amino acid sequence of SEQ ID NO: 9;
wherein the spFv chain comprises a light chain CDR1 comprising the amino acid sequence of RARQSIGTAIH (SEQ ID NO: 24), a light chain CDR2 comprising the amino acid sequence of YASESIS (SEQ ID NO: 25), a light chain CDR3 comprising the amino acid sequence of QQSGSWPYT (SEQ ID NO: 26), a heavy chain CDR1 comprising the amino acid sequence of RYNMN (SEQ ID NO: 73), a heavy chain CDR2 comprising the amino acid sequence of SISTSSNYIYYADSVKG (SEQ ID NO: 74), and a heavy chain CDR3 comprising the amino acid sequence of GWGPFDY (SEQ ID NO: 22);
wherein the heavy chain associates with the light to form an antigen binding site that immunospecifically binds to a mutant calreticulin (CALRmut); and
wherein the spFv chain immunospecifically binds to CD3.

In some embodiments, the bispecific antibody comprises a heavy chain, a light chain, and a spFv chain,
wherein the heavy chain comprises a heavy chain CDR1 comprising the amino acid sequence of SEQ ID NO: 70, a heavy chain CDR2 comprising the amino acid sequence of SEQ ID NO: 71, and a heavy chain CDR3 comprising the amino acid sequence of SEQ ID NO: 72;
wherein the light chain comprises a light chain CDR1 comprising the amino acid sequence of SEQ ID NO: 66, a light chain CDR2 comprising the amino acid sequence of DAS, and a light chain CDR3 comprising the amino acid sequence of SEQ ID NO: 9;
wherein the spFv chain comprises a light chain CDR1 comprising the amino acid sequence of QSIGTA (SEQ ID NO: 81), a light chain CDR2 comprising the amino acid sequence of YAS, a light chain CDR3 comprising the amino acid sequence of QQSGSWPYT (SEQ ID NO: 26), a heavy chain CDR1 comprising the amino acid sequence of GFTFSRYN (SEQ ID NO: 78), a heavy chain CDR2 comprising the amino acid sequence of ISTSSNYI (SEQ ID NO: 79), and a heavy chain CDR3 comprising the amino acid sequence of TRGWGPFDY (SEQ ID NO: 80);
wherein the heavy chain associates with the light to form an antigen binding site that immunospecifically binds to a mutant calreticulin (CALRmut); and
wherein the spFv chain immunospecifically binds to CD3

In some embodiments, the heavy chain CDR1 consists of the amino acid sequence of SEQ ID NO: 11, the heavy chain CDR2 consists of the amino acid sequence of SEQ ID NO: 12, and the heavy chain CDR3 consists of the amino acid sequence of SEQ ID NO: 13. In some embodiments, the light chain CDR1 consists of the amino acid sequence of SEQ ID NO: 7, the light chain CDR2 consists of the amino acid sequence of SEQ ID NO: 8, and the light chain CDR3 consists of the amino acid sequence of SEQ ID NO: 9. In some embodiments, the spFv light chain CDR1 consists of the amino acid sequence of SEQ ID NO: 24, the spFv light chain CDR2 consists of the amino acid sequence of SEQ ID NO: 25, the spFv light chain CDR3 consists of the amino acid sequence of SEQ ID NO: 26, the spFv heavy chain CDR1 consists of the amino acid sequence of SEQ ID NO: 20, the spFv heavy chain CDR2 consists of the amino acid sequence of SEQ ID NO: 21, and the spFv heavy chain CDR3 consists of the amino acid sequence of SEQ ID NO: 22.

In some embodiments, the heavy chain CDR1 consists of the amino acid sequence of SEQ ID NO: 68, the heavy chain CDR2 consists of the amino acid sequence of SEQ ID NO: 69, and the heavy chain CDR3 consists of the amino acid sequence of SEQ ID NO: 13. In some embodiments the light chain CDR1 consists of the amino acid sequence of SEQ ID NO: 7, the light chain CDR2 consists of the amino acid sequence of SEQ ID NO: 8, and the light chain CDR3 consists of the amino acid sequence of SEQ ID NO: 9. In some embodiments, the spFv light chain CDR1 consists of SEQ ID NO: 24, the spFv light chain CDR2 consist of the amino acid sequence of SEQ ID NO: 25, the spFv light chain CDR3 consists of the amino acid sequence of SEQ ID NO: 26, the spFv heavy chain CDR1 consists of the amino acid sequence of SEQ ID NO: 75, the spFv heavy chain CDR2 consists of the amino acid sequence of SEQ ID NO: 76, and the spFv heavy chain CDR3 consists of the amino acid sequence of SEQ ID NO: 22.

In some embodiments, the heavy chain CDR1 consists of the amino acid sequence of SEQ ID NO: 10, the heavy chain CDR2 consists of the amino acid sequence of SEQ ID NO: 67, and the heavy chain CDR3 consists of the amino acid sequence of SEQ ID NO: 13. In some embodiments the light chain CDR1 consists of the amino acid sequence of SEQ ID NO: 7, the light chain CDR2 consists of the amino acid sequence of SEQ ID NO: 8, and the light chain CDR3 consists of the amino acid sequence of SEQ ID NO: 9. In some embodiments, the spFv light chain CDR1 consists of SEQ ID NO: 24, the spFv light chain CDR2 consist of the amino acid sequence of SEQ ID NO: 25, the spFv light chain CDR3 consists of the amino acid sequence of SEQ ID NO: 26, the spFv heavy chain CDR1 consists of the amino acid sequence of SEQ ID NO: 73, the spFv heavy chain CDR2 consists of the amino acid sequence of SEQ ID NO: 74, and the spFv heavy chain CDR3 consists of the amino acid sequence of SEQ ID NO: 22.

In some embodiments, the heavy chain CDR1 consists of the amino acid sequence of SEQ ID NO: 70, the heavy chain CDR2 consists of the amino acid sequence of SEQ ID NO: 71, and the heavy chain CDR3 consists of the amino acid sequence of SEQ ID NO: 72. In some embodiments the light chain CDR1 consists of the amino acid sequence of SEQ ID NO: 66, the light chain CDR2 consists of the amino acid sequence of DAS, and the light chain CDR3 consists of the amino acid sequence of SEQ ID NO: 9. In some embodiments, the spFv light chain CDR1 consists of SEQ ID NO: 81, the spFv light chain CDR2 consist of the amino acid sequence of YAS, the spFv light chain CDR3 consists of the amino acid sequence of SEQ ID NO: 26, the spFv heavy chain CDR1 consists of the amino acid sequence of SEQ ID NO: 78, the spFv heavy chain CDR2 consists of the amino acid sequence of SEQ ID NO: 79, and the spFv heavy chain CDR3 consists of the amino acid sequence of SEQ ID NO: 80.

In some embodiments, the heavy chain of the bispecific antibody comprises a VH comprising an amino acid sequence that is at least 90%, 91%, 92%, 93%, 94%, 95%, 96%, 97%, 98%, or 99% identical to the amino acid sequence of SEQ ID NO: 14. In some embodiments, the heavy chain VH comprises the amino acid sequence of SEQ ID NO: 14. In some embodiments, the heavy chain VH consists of the amino acid sequence of SEQ ID NO: 14. In some embodiments, the light chain of the bispecific antibody, or bispecific antigen-binding fragment thereof, comprises a light chain VL comprising an amino acid sequence that is at least 90%, 91%, 92%, 93%, 94%, 95%, 96%, 97%, 98%, or 99% identical to the amino acid sequence of SEQ ID NO: 16. In some embodiments, the light chain VL comprises the amino acid sequence of SEQ ID NO: 16. In some embodiments, the light chain VL consists of the amino acid sequence of SEQ ID NO: 16.

In some embodiments, the spFv chain of the bispecific antibody, or bispecific antigen-binding fragment thereof, comprises a spFv VH comprising an amino acid sequence that is at least 90%, 91%, 92%, 93%, 94%, 95%, 96%, 97%, 98%, or 99% identical to the amino acid sequence of SEQ ID NO: 23. In some embodiments, the spFv VH comprises the amino acid sequence of SEQ ID NO: 23. In some embodiments, the spFv VH consists of the amino acid sequence of SEQ ID NO: 23. In some embodiments, the spFv chain of the bispecific antibody, or bispecific antigen-binding fragment thereof, comprises a spFv VL comprising an amino acid sequence that is at least 90%, 91%, 92%, 93%, 94%, 95%, 96%, 97%, 98%, or 99% identical to the amino acid sequence of SEQ ID NO: 27. In some embodiments, the spFv VL comprises the amino acid sequence of SEQ ID NO: 27. In some embodiments, the spFv VL consists of the amino acid sequence of SEQ ID NO: 27.

In some embodiments, the bispecific antibody comprises a heavy chain VH comprising the amino acid sequence of SEQ ID NO: 14, and a light chain VL comprising the amino acid sequence of SEQ ID NO: 16. In some embodiments, the bispecific antibody comprises a heavy chain VH consisting of the amino acid sequence of SEQ ID NO: 14, and a light chain VL consisting of the amino acid sequence of SEQ ID NO: 16.

In some embodiments, the bispecific antibody comprises a heavy chain VH comprising the amino acid sequence of SEQ ID NO: 14, a light chain VL comprising the amino acid sequence of SEQ ID NO: 16, a spFv VH comprising the amino acid sequence of SEQ ID NO: 23, and a spFv VL comprising the amino acid sequence of SEQ ID NO: 27. In some embodiments, the bispecific antibody comprises a heavy chain VH consisting of the amino acid sequence of SEQ ID NO: 14, a light chain VL consisting of the amino acid sequence of SEQ ID NO: 16, a spFv VH consisting of the amino acid sequence of SEQ ID NO: 23, and a spFv VL consisting of the amino acid sequence of SEQ ID NO: 27.

In some embodiments, the bispecific antibody is an IgG. In some embodiments, the bispecific antibody is an IgG1, IgG2, IgG3, or IgG4 isotype. In some embodiments, the bispecific antibody is an IgG1 isotype, or comprises an IgG1 isotype Fc region.

In some embodiments, the bispecific antibody further comprises L234A, L235A, and D265S substitutions in the Fc region. In some embodiments, these substitutions, known as AAS substitutions, in the Fc region are known to limit or abolish antibody interactions with Fc receptors.

In some embodiments, the bispecific antibody further comprises knob-into-hole (KiH) substitution in the Fc region. One Fc region comprises an T366W substitution (the “knob” Fc region), while the other Fc region comprises T366S, L368A, and Y407V substitutions (the “hole” Fc region).

In some embodiments, the bispecific antibody further comprises H435R and Y436F substitutions in the Fc region. In some embodiments, these substitutions in the Fc region are known to disrupt protein A binding of monomeric and homodimerized of “hole” Fc regions.

In some embodiments, the bispecific antibody comprises a heavy chain comprising an amino acid sequence that is at least 90%, 91%, 92%, 93%, 94%, 95%, 96%, 97%, 98%, or 99% identical to the amino acid sequence of SEQ ID NO: 15. In some embodiments, the heavy chain comprises the amino acid sequence of SEQ ID NO: 15. In some embodiments, the heavy chain consists of the amino acid sequence of SEQ ID NO: 15.

In some embodiments, the bispecific antibody comprises a light chain comprising an amino acid sequence that is at least 90%, 91%, 92%, 93%, 94%, 95%, 96%, 97%, 98%, or 99% identical to the amino acid sequence of SEQ ID NO: 17. In some embodiments, the heavy chain comprises the amino acid sequence of SEQ ID NO: 17. In some embodiments, the heavy chain consists of the amino acid sequence of SEQ ID NO: 17.

In some embodiments, the bispecific antibody comprises a spFv chain comprising an amino acid sequence that is at least 90%, 91%, 92%, 93%, 94%, 95%, 96%, 97%, 98%, or 99% identical to the amino acid sequence of SEQ ID NO: 28. In some embodiments, the spFv chain comprises the amino acid sequence of SEQ ID NO: 28. In some embodiments, the spFv chain consists of the amino acid sequence of SEQ ID NO: 28.

In some embodiments, the bispecific antibody comprises a heavy chain comprising the amino acid sequence of SEQ ID NO: 15, a light chain comprising the amino acid sequence of SEQ ID NO: 17, and a spFv chain comprising the amino acid sequence of SEQ ID NO: 28, wherein the heavy chain associates with the light chain to form an antigen binding site that immunospecifically binds to a mutant calreticulin (CALRmut); and wherein the spFv chain immunospecifically binds to CD3.

In some embodiments, the bispecific antibody comprises a heavy chain consisting of the amino acid sequence of SEQ ID NO: 15, a light chain consisting of the amino acid sequence of SEQ ID NO: 17, and a spFv chain consisting of the amino acid sequence of SEQ ID NO: 28, wherein the heavy chain associates with the light chain to form an antigen binding site that immunospecifically binds to a mutant calreticulin (CALRmut); and wherein the spFv chain immunospecifically binds to CD3.

### Vectors, Host Cells, and Antibody Production

The antibodies disclosed herein, including but not limited to the bispecific antibody disclosed herein, can be produced from a host cell. A host cell refers to a vehicle that includes the necessary cellular components, e.g., organelles, needed to express the polypeptides and constructs described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, etc.). The choice of nucleic acid vectors depends in part on the host cells to be used. Generally, host cells are of either prokaryotic (e.g., bacterial) or eukaryotic (e.g., mammalian) origin.

### Nucleic Acid Vector Construction and Host Cells

A nucleic acid sequence encoding the amino acid sequence of the antibodies disclosed herein, including but not limited to the bispecific antibody disclosed herein, may be prepared by a variety of methods known in the art. These methods include, but are not limited to, oligonucleotide-mediated (or site-directed) mutagenesis and PCR mutagenesis. A nucleic acid molecule encoding an antibody disclosed herein may be obtained using standard techniques, e.g., gene synthesis. Alternatively, a nucleic acid molecule encoding an antibody disclosed herein may be mutated to contain specific amino acid substitutions using standard techniques in the art, e.g., QuikChange™ mutagenesis. Nucleic acid molecules can be synthesized using a nucleotide synthesizer or PCR techniques.

In some embodiments, the nucleotide sequence encoding the anti-CALRmut heavy chain comprises SEQ ID NO: 18.

In some embodiments, the nucleotide sequence encoding the anti-CALRmut light chain comprises SEQ ID NO: 19.

In some embodiments, the nucleotide sequence encoding the anti-CD3 spFv chain comprises SEQ ID NO: 29.

Nucleic acid sequences encoding the disclosed antibodies can be inserted into a vector capable of replicating and expressing the nucleic acid molecules in prokaryotic or eukaryotic host cells. Many vectors are available in the art and can be used for the purpose of the disclosure. Each vector may contain various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells are used as host cells for the disclosure. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7030, and HsS78Bst cells. In other embodiments, E. coli cells are used as host cells for the disclosure. Examples of E. coli strains include, but are not limited to, E. coli 294 (ATCC® 31,446), E. coli, 1776 (ATCC® 31,537, E. coli BL21 (DE3) (ATCC® BAA-1025), and E. coli RV308 (ATCC® 31,608). Different host cells have characteristic and specific mechanisms for the posttranslational processing and modification of protein products. Appropriate cell lines or host systems may be chosen to ensure the correct modification and processing of the antibody expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 (Jul. 20, 2004) and Vladimir Voynov and Justin A. Caravella (eds.) Therapeutic Proteins: Methods and Protocols (Methods in Molecular Biology) Humana Press; 2nd ed. 2012 (Jun. 28, 2012). In certain embodiments, the antibodies of the disclosure are isolated from at least one component of a sample (e.g., a cell culture) from which the antibody is obtained.

### Pharmaceutical Compositions, Preparations, and Kits

The disclosure features pharmaceutical compositions that include one or more bispecific anti-antibodies described herein. In addition to a therapeutically effective amount of the antibody, the pharmaceutical compositions can contain one or more pharmaceutically acceptable carriers or excipients, which can be formulated by methods known to those skilled in the art.

Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

In some embodiments, any bispecific antibody disclosed herein can be packaged in a kit. In some embodiments, the kit comprises a polynucleotide encoding any antibody, or antigen-binding fragment, disclosed herein. In some embodiments, the kit comprises any pharmaceutical composition comprising any antibody, or antigen-binding fragment, disclosed herein. In some embodiments, the kit comprises one or more of any bispecific antibody disclosed herein, any pharmaceutical composition disclosed herein, a polynucleotide disclosed herein, or any combination thereof.

### Methods of Treatment and Indications

In some embodiments, an anti-CALRmut/anti-CD3 bispecific antibody described herein is administered as part of a pharmaceutical composition. In some embodiments, the pharmaceutical composition comprises a therapeutically effective amount of the bispecific antibody.

As used herein, the term “therapeutically effective amount” refers to an amount, e.g., pharmaceutical dose, effective in inducing a desired biological effect in a subject or patient or in treating a patient having a condition or disorder described herein. It is also to be understood that a “therapeutically effective amount” may be interpreted as an amount giving a desired therapeutic effect, either taken in one dose or in any dosage or route, taken alone or in combination with other therapeutic agents.

As used herein, the term “no more than” refers to an amount that is less than or equal to. This may be an amount in integers. For example, no more than two substitutions can refer to 0, 1, or 2 substitutions.

As used herein, the terms “treatment” or “treating” refer to reducing, decreasing, decreasing the risk of, or decreasing the side effects of a particular disease or condition. Reducing, decreasing, decreasing the risk of, or decreasing the side effects of are relative to a subject who did not receive treatment, e.g., a control, a baseline, or a known control level or measurement.

In some embodiments, a method of redirecting a T cell to a cell expressing a mutant calreticulin is provided, the method comprising administering to a subject in need thereof a therapeutically effective amount of any antibody, or antigen-binding fragment thereof, disclosed herein to redirect a T cell to the cell expressing a mutant calreticulin.

Myeloproliferative neoplasms (MPNs) are a group of rare blood cancers in which abnormal and uncontrolled excess red blood cells, white blood cells or platelets are produced in the bone marrow. In some embodiments, the MPN is selected from the group consisting of chronic myelogenous leukemia, polycythemia vera, primary myelofibrosis, secondary myelofibrosis, secondary acute myeloid leukemia, essential thrombocythemia, chronic neutrophilic leukemia, and chronic eosinophilic leukemia.

MPNs are characterized by cytokine-independent activation of the Janus kinase (JAK)—signal transducer and activator of transcription (STAT) signaling pathway. Somatic mutations in hematopoietic stem cells in JAK2, thrombopoietin receptor (also known as MPL), and CALR (calreticulin) are phenotypic drivers in the pathogenesis of MPN. These mutations lead to pathological and constitutive activation of the JAK2-STAT5 pathway, resulting in clonal expansion of myeloid progenitors that no longer need cytokine activation for cell proliferation and differentiation. CALR is the second most frequently mutated gene, after JAK2, in MPN. Thus, in some embodiments, MPN is characterized by the presence of a mutant calreticulin (CALRmut).

CALR wild-type protein is a major chaperone and Ca2+-binding protein of the endoplasmic reticulum (ER). It plays major roles in protein folding, regulation of cell survival, and immune response. Its protein structure has 3 domains: (i) an amino domain, which is essential for chaperone function via its lectin binding sites and contains an ER signal peptide sequence, (ii) a proline-rich P domain, which binds to calcium and has a chaperone lectin-binding site, (iii) and a carboxyl domain, which also functions in calcium binding and includes an ER-retention signal (KDEL motif).

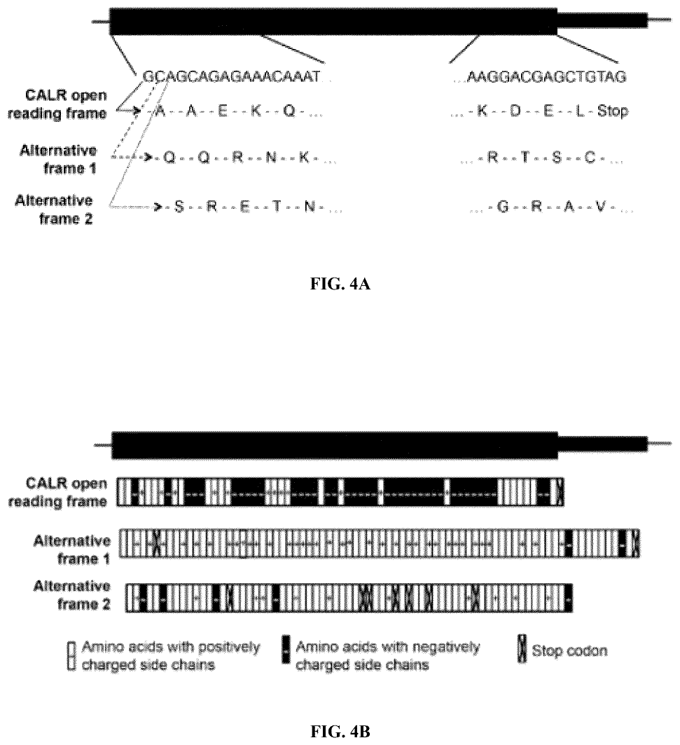

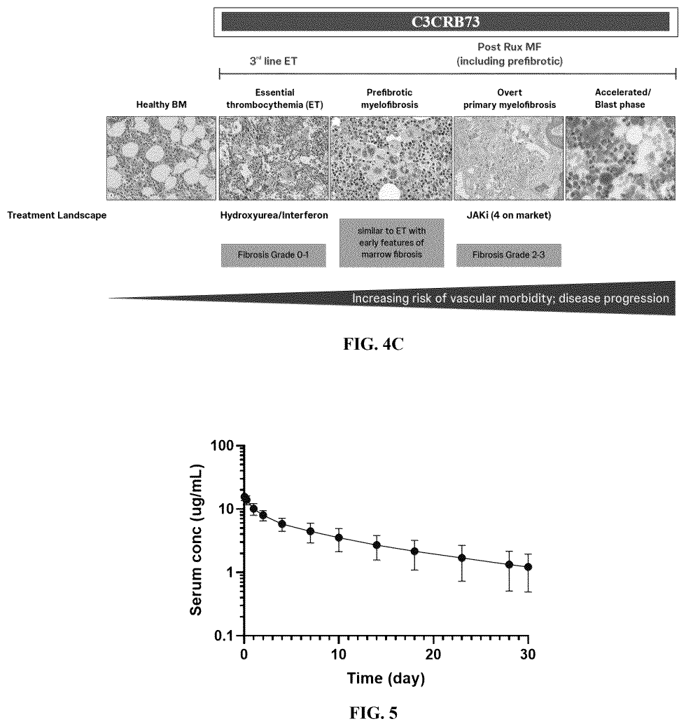

All CALR mutations are insertions or deletions resulting in a frameshift in the last exon (i.e., Exon 9) of the gene, resulting in loss of the KDEL motif and generation of a mutant-specific 36 amino acid positively charged C terminal tail (shared between the majority of known CALR mutations) (see FIG. 4A and FIG. 4B). Due to loss of the KDEL motif, CALRmut is not confined to the ER and through interaction of MPL is trafficked to the cell surface. Thus far, more than 50 different types of mutations in CALR have been detected, but a 52 base pair deletion (CALRdel52; Type 1 mutation) and a 5 base pair insertion (CALRins5; Type 2 mutation) are the most frequent types, overall being found in more than 80% of all patients with CALRmut. The other mutations include type 1 like or type 2 like, depending on the extent of amino acid deletion. See, e.g., Pietra et al. Leukemia. 2016 February; 30(2):431, on mutation subtypes in CALR-mutant myeloproliferative neoplasms, the content of which is incorporated herein by reference in its entirety. The spectrum of CALRmut myeloproliferative disease can be defined by CALRmut, degree of fibrosis and blood counts and their treatments (FIG. 4C).

Exemplary C-terminal amino acid residues/sequences of mutant calreticulin proteins comprise the amino acid sequences of SEQ ID NOs: 30-65.

It is envisaged that the herein provided antibodies can specifically bind to a fragment or part of the C-terminus of mutant calreticulin protein. It is preferred that the herein provided antibodies specifically bind to RRKMSPARPRTSCREACLQGWTEA (SEQ ID NO: 2).

CALRmut lacks a transmembrane domain and requires binding to the extracellular domain of MPL for activation of downstream signaling and for its oncogenic transformation. CALRmut binding to MPL depends on binding to both immature asparagine-linked glycan at residue N117 and to several negatively charged patches of MPL in the ER. This leads to the formation of a tight complex and charge-driven interactions providing the specificity of CALRmut for MPL. CALRmut protects N117-linked glycans of MPL from further processing in the Golgi apparatus. The tetrameric complex formed between 2 CALRmut and 2 MPL proteins is shuttled through the Golgi apparatus to the cell membrane, leading to constitutive activation of downstream kinase JAK2. MPL in complex with CALRmut remains in an immature glycosylation status on the cell surface. Recent genetic and focused pharmacological screens identified the N-glycosylation pathway, more particularly immature glycosylation of MPL, as essential for CALRmut-driven oncogenesis. In contrast in CALR wild-type cells, MPL is expressed in a mature glycosylation state on the cell membrane that cannot be bound or stimulated by soluble CALRmut.

In some embodiments, a method of inhibiting the growth or proliferation of a myeloproliferative neoplasm (MPN) is provided, the method comprising administering to a subject in need thereof a therapeutically effective amount of the bispecific antibody, or bispecific antigen-binding fragment thereof, disclosed herein to inhibit the growth or proliferation of the MPN.

In some embodiments, a method of treating a MPN is provided, the method comprising administering to a subject in need thereof any antibody, or antigen-binding fragment thereof, disclosed herein to the subject for a time sufficient to treat the cancer.

In some embodiments, the subject is diagnosed with essential thrombocythemia or myelofibrosis.

“Essential thrombocythemia” or “ET” as used herein is an MPN that is characterized by megakaryocyte proliferation in the bone marrow which results in thrombocythemia, an elevated concentration of platelets in the peripheral circulation. The presence of CALRmut in peripheral blood mononuclear cells (PBMNCs) has been used for diagnosis (WHO Classification of Tumours Editorial Board 2022) and account for up to 25% of cases of ET (Klampfl 2013, N Engl J Med. 369:2379-2390).

Hydroxyurea-resistant population of ET patients (resistant to cytoreductive therapy with hydroxyurea) already at high vascular risk represent a patient population without a clear standard of care for whom disease-modifying therapy may yield particular benefit. Indeed, HU-resistance in this high vascular risk group is associated with an increased progression of disease and reduced overall survival, with a cumulative 10-year risk progression of 13% and transformation to leukemia of 8.5% (Hernandez-Boluda 2011, Br J Haematol. 152(1):81-88)

In certain embodiments, the subject is resistant to cytoreductive therapy with hydroxyurea.

In certain embodiments, a method of the application is used to treat ET, particularly an ET with high-risk of thrombosis or hemorrhage and intolerant or resistant or refractory to hydroxyurea. Preferably, the method results in at least one of: 1) normal spleen size on imaging; and 2) platelet count ≤400×10⁹/L and/or white cell count ≤10×10⁹/L in peripheral blood.

“Primary Myelofibrosis” or “PMF” as used herein is characterized by the proliferation of megakaryocytes and granulocytes in the bone marrow. This increase in marrow fibrosis and osteosclerosis drives extra-medullary hematopoiesis, which leads to significant hepato-splenomegaly. Splenomegaly is common, progressive, and often massive. PMF is associated with a greater burden of concomitant genetic mutations and chromosomal abnormalities than ET, which in turn predispose to disease progression and leukemic transformation. Symptoms of PMF derive from a hypermetabolic state, fatigue, anorexia, weight loss, as well as symptoms attributed to splenomegaly including abdominal discomfort and early satiety. PMF risk-stratification can be performed using the IPSS at diagnosis (Cervantes 2009, Blood. 113:2895-2901). The Dynamic International Prognostic Scoring System (DIPSS) can be used to predict survival during follow-up by incorporating prognostic information from karyotype, platelet count, and transfusion status. The DIPSS is described in Passamonti 2010, Blood. 115:1703-1708), the relevant content of which is incorporated herein by reference. In some embodiment, a method of the application is used to treat a primary MF with a DIPSS risk score of Intermediate 1 (Int-1), Intermediate 2 (Int-2) or High-Risk (HR). In some embodiment, the subject with primary MF has a blast percentage not consistently exceeding 20% in blood or bone marrow.

“Post-essential thrombocythemia Myelofibrosis” or “post-ET MF” refers to secondary MF from the progression of ET. The median time to progression in CALRmut ET was 12.1 years, with a 15-year cumulative incidence of 13.4% (Mora 2018, Leuk Res. 69:100-102). The Myelofibrosis Secondary to PV and ET-Prognostic Model (MYSEC-PM) has defined 4 risk categories based on overall survival. The MYSEC-PM is described in Passamonti 2017, Leukemia. 31:2726-2731, the relevant content of which is incorporated herein by reference. In some embodiment, a method of the application is used to treat a post-ET MF, such as a post-ET MF with a MYSEC-PM risk score of Int-1, Int-2 or HR. In some embodiment, the subject with post-ET MF has a blast percentage not consistently exceeding 20% in blood or bone marrow.

In certain embodiments, a method of the application is used to treat MF in a subject in need thereof, such as a PMF or post-ET MF. The method results in a reduction in splenic volume of a subject with MF compared to a baseline splenic volume measured before administration of the anti-mutant calreticulin (CALRmut)/anti-CD3 bispecific antibody, preferably the splenic volume is reduced by at least 35%, such 35%, 40%, 45%, and 50% or any value in between, as compared to a baseline splenic volume measured before administration of the anti-mutant calreticulin (CALRmut)/anti-CD3 bispecific antibody. In certain embodiments, the treatment results in at least one of 1) bone marrow age-adjusted normocellularity, 2) <5% blasts, and 3) Hemoglobin ≥10 g/dL, neutrophil count ≥1×10⁹/L, and/or platelet count ≥100×10⁹/L in peripheral blood. In some embodiments, the method modifies the MF disease, e.g., it exerts a clinically meaningful impact on survival outcomes and/or restoration of normal hematopoiesis in the subject in conjunction with improvement in bone marrow fibrosis through a substantial and durable reduction in the clonal burden of disease. See, e.g., Pemmaraju et al 2022, J Clin Oncol. 40(26):3032-3036, for disease modification in MF, the relevant content of which is incorporated herein by reference in its entirety.

In certain embodiments, a method of the application results in a decreases soluble CALRmut levels in serum of the subject, preferably the soluble CALRmut levels are reduced by at least 50%, such as 50%, 55%, 65%, 70%, 75%, 80%, 85%, 90%, 95%, 100%, or any value in between, compared to a baseline level measured before administration of the anti-mutant calreticulin (CALRmut)/anti-CD3 bispecific antibody. In some embodiment, the method results in a reduction of CALRmut positive cells in bone marrow of the subject. In some embodiment, the method results in improved bone marrow architecture, such as reduction in bone marrow reticulin fibrosis and bone marrow cellularity.

In certain embodiments, the subject is ineligible, intolerant or resistant to JAK inhibitor therapy. Ineligible indicates that JAKi is contraindicated due to prior history of severe infections such as tuberculosis, progressive multifocal leukoencephalopathy, and skin malignancies that are known to be associated or exacerbated by JAKi, or other significant considerations as documented by the treating physician. In tolerant indicates hematologic toxicity such as platelet count <50×10⁹/L and/or neutrophils ≤0.5×10⁹/L despite recommended dose adjustments and interruptions. Evidence of resistant and/or refractory would be persistent splenomegaly, lack of symptom improvement, persistent leukoerythroblastosis, anemia <10 g/dL or leukocytosis >11×10⁹/L after administration with JAK inhibitor.

In certain embodiments, the subject has been administered a prior therapy. In certain embodiments, wherein the subject is diagnosed with essential thrombocythemia, the prior therapy comprises at least 2 lines of prior cytoreductive therapy, at least 1 of which must have been hydroxyurea. In certain embodiments, wherein the subject is diagnosed with myelofibrosis, the prior therapy comprises at least 1 prior JAK inhibitor (JAKi) therapy. In certain embodiments, prior therapy comprises Interferon-α (pegylated or standard preparation), anagrelide, busulfan. In other embodiments, the prior therapy comprises JAKi, immunomodulatory drug therapy (such as thalidomide), danazol, or other therapy intended to lead to disease modification.

### EXAMPLES

### Example 1: CALRmut x CD3 Bispecific IgG Antibody Profile

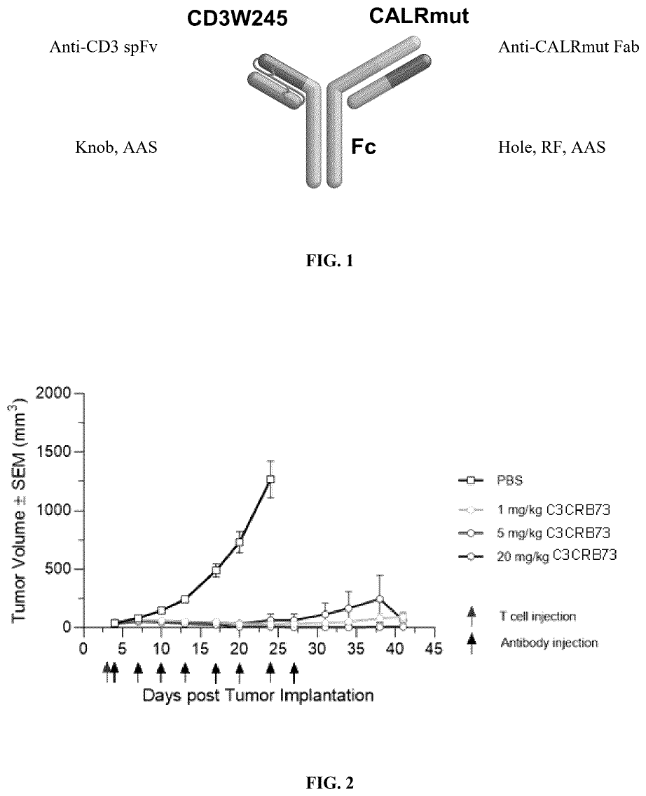

FIG. 1 shows the general molecular architecture of an exemplary bispecific molecule against mutant calreticulin (CALRmut) and CD3 (CALRmut x CD3) useful for the invention described herein. In particular, C3CRB73 is an IgG1-based bispecific antibody that can simultaneously bind to CD3e (CD3R; Uniprot ID: P07766) on T cells and to CALRmut on tumor cells. The antibody features mutations of L234A, L235A, and D265S (i.e., AAS) in the fragment crystallizable (Fc) constant region to abolish interaction with Fc receptors, and heterodimerization is enhanced using the knob-into-hole (KiH) mutations. It comprises an anti-CD3ε spFv fused to the N terminus of the ‘knob’ Fc region (i.e., T366W). The ‘hole’ chain (i.e., T366S, L368A, Y407V) features an anti-CALRmut Fab and contains ‘RF’ mutations (i.e., H435R, Y436F) to disrupt Protein A binding of monomeric and homodimerized hole chains.

### A. Structure

C3CRB73 was generated by co-expression of the anti-CD3ε scFv “knob” heavy chain (HC) with the anti-CALRmut Fab heavy chain containing the “hole” and RF mutations paired with its cognate light chain (LC). The amino acid sequences for C3CRB73 heavy chains and light chain, as deduced from the cDNA sequence and confirmed by peptide mapping and mass spectrometry, are shown below with complementarity-determining regions (CDRs) listed according to AbM, Chothia, Kabat or IMGT definition. The CD3 heavy chain sequence includes the stapled CD3 ‘spFv’ arm. The heterodimerization mutations (KiH, T366W in the CD3 spFv arm and T366S, L368A and Y407V in the CALRmut heavy chain) and H435R and Y436F mutations in the CALRmut heavy chain are present, as is the N104S mutation in CDR-H3 in the CALRmut heavy chain.

TABLE 1
Amino Acid Sequences for C3CRB73, a CALRmut x CD3 Bispecific Antibody

| SEQ ID | | |
|---|---|---|
| NO | Description | Sequence |
| 11 | CALRmut Heavy CDR1 AbM | GIDLSNNAIS |
| 12 | CALRmut Heavy CDR2 AbM | VIGNTGDTY |
| 13 | CALRmut Heavy CDR3 AbM | GPPSYSSSVKNI |
| 68 | CALRmut Heavy CDR1 Chothia | GIDLSNN |
| 69 | CALRmut Heavy CDR2 Chothia | GNTGD |
| 13 | CALRmut Heavy CDR3 Chothia | GPPSYSSSVKNI |
| 10 | CALRmut Heavy CDR1 Kabat | NNAIS |
| 67 | CALRmut Heavy CDR2 Kabat | VIGNTGDTYYADSAKG |
| 13 | CALRmut Heavy CDR3 Kabat | GPPSYSSSVKNI |
| 70 | CALRmut Heavy CDR1 IMGT | GIDLSNNA |
| 71 | CALRmut Heavy CDR2 IMGT | IGNTGDT |
| 72 | CALRmut Heavy CDR3 IMGT | VRGPPSYSSSVKNI |
| 14 | CALRmut VH | EVQLLESGGGLVQPGGSLRLSCAVSGIDLSNNAISWV |
| | | RQAPGKGLEYVGVIGNTGDTYYADSAKGRFTISRDSS |
| | | KTTLYLQMNSLRAEDTAVYFCVRGPPSYSSSVKNIWG |
| | | QGTLVTVSS |
| 15 | CALRmut Heavy Chain | EVQLLESGGGLVQPGGSLRLSCAVSGIDLSNNAISWV |
| | | RQAPGKGLEYVGVIGNTGDTYYADSAKGRFTISRDSS |
| | | KTTLYLQMNSLRAEDTAVYFCVRGPPSYSSSVKNIWG |
| | | QGTLVTVSSASTKGPSVFPLAPSSKSTSGGTAALGCLV |
| | | KDYFPEPVTVSWNSGALTSGVHTFPAVLQSSGLYSLS |
| | | SVVTVPSSSLGTQTYICNVNHKPSNTKVDKKVEPKSC |
| | | DKTHTCPPCPAPEAAGGPSVFLFPPKPKDTLMISRTPE |
| | | VTCVVVSVSHEDPEVKFNWYVDGVEVHNAKTKPREE |
| | | QYNSTYRVVSVLTVLHQDWLNGKEYKCKVSNKALP |
| | | APIEKTISKAKGQPREPQVYTLPPSREEMTKNQVSLSC |
| | | AVKGFYPSDIAVEWESNGQPENNYKTTPPVLDSDGSF |
| | | FLVSKLTVDKSRWQQGNVFSCSVMHEALHNRFTQKS |
| | | LSLSPGK |
| 7 | CALRmut Light CDR1 AbM | QSSQSVYNNNWLS |
| 8 | CALRmut Light CDR2 AbM | DASKLES |
| 9 | CALRmut Light CDR3 AbM | AGGFTGNVYT |
| 7 | CALRmut Light CDR1 Chothia | QSSQSVYNNNWLS |
| 8 | CALRmut Light CDR2 Chothia | DASKLES |
| 9 | CALRmut Light CDR3 Chothia | AGGFTGNVYT |
| 7 | CALRmut Light CDR1 Kabat | QSSQSVYNNNWLS |
| 8 | CALRmut Light CDR2 Kabat | DASKLES |
| 9 | CALRmut Light CDR3 Kabat | AGGFTGNVYT |
| 66 | CALRmut Light CDR1 IMGT | QSSQSVYNNNWLS |
| — | CALRmut Light CDR2 IMGT | DAS |
| 9 | CALRmut Light CDR3 IMGT | AGGFTGNVYT |
| 16 | CALRmut VL | DIQMTQSPSSVSASVGDRVTITCQSSQSVYNNNWLSW |
| | | LQQKPGKAPKRLIYDASKLESGVPSRFSGSGSGTDFTL |
| | | TISSVQPEDAATYYCAGGFTGNVYTFGGGTKVEIK |
| 17 | CALRmut Light Chain | DIQMTQSPSSVSASVGDRVTITCQSSQSVYNNNWLSW |
| | | LQQKPGKAPKRLIYDASKLESGVPSRFSGSGSGTDFTL |
| | | TISSVQPEDAATYYCAGGFTGNVYTFGGGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| 20 | CD3 spFv Heavy CDR1 AbM | GFTFSRYNMN |
| 21 | CD3 spFv Heavy CDR2 AbM | SISTSSNYIY |
| 22 | CD3 spFv Heavy CDR3 AbM | GWGPFDY |
| 75 | CD3 spFv Heavy CDR1 Chothia | GFTFSRY |
| 76 | CD3 spFv Heavy CDR2 Chothia | STSSNY |
| 22 | CD3 spFv Heavy CDR3 Chothia | GWGPFDY |
| 73 | CD3 spFv Heavy CDR1 Kabat | RYNMN |
| 74 | CD3 spFv Heavy CDR2 Kabat | SISTSSNYIYYADSVKG |
| 22 | CD3 spFv Heavy CDR3 Kabat | GWGPFDY |
| 78 | CD3 spFv Heavy CDR1 IMGT | GFTFSRYN |
| 79 | CD3 spFv Heavy CDR2 IMGT | ISTSSNYI |
| 80 | CD3 spFv Heavy CDR3 IMGT | TRGWGPFDY |
| 23 | CD3 spFv VH | EVQLVESGGGLVKPGGSLRLSCAASGFTFSRYNMNW |
| | | VRQAPGKGLEWVSSISTSSNYIYYADSVKGRFTFSRD |
| | | NAKNSLDLQMSGLRAEDTAIYYCTRGWGPFDYWGC |
| | | GTLVTVSS |
| 24 | CD3 spFv Light CDR1 AbM | RARQSIGTAIH |
| 25 | CD3 spFv Light CDR2 AbM | YASESIS |
| 26 | CD3 spFv Light CDR3 AbM | QQSGSWPYT |
| 24 | CD3 spFv Light CDR1 Chothia | RARQSIGTAIH |
| 25 | CD3 spFv Light CDR2 Chothia | YASESIS |
| 26 | CD3 spFv Light CDR3 Chothia | QQSGSWPYT |
| 24 | CD3 spFv Light CDR1 Kabat | RARQSIGTAIH |
| 25 | CD3 spFv Light CDR2 Kabat | YASESIS |
| 26 | CD3 spFv Light CDR3 Kabat | QQSGSWPYT |
| 81 | CD3 spFv Light CDR1 IMGT | QSIGTA |
| — | CD3 spFv Light CDR2 IMGT | YAS |
| 26 | CD3 spFv Light CDR3 IMGT | QQSGSWPYT |
| 27 | CD3 spFv VL | DIQMTQSPSSLSASVGDRVTITCRARQSIGTAIHWYQQ |
| | | KPGCAPKLLIKYASESISGVPSRFSGSGSGTDFTLTISSL |
| | | QPEDFATYYCQQSGSWPYTFGQGTKLEIK |
| 82 | CD3 spFv VL-VH | DIQMTQSPSSLSASVGDRVTITCRARQSIGTAIHWYQQ |
| | | KPGCAPKLLIKYASESISGVPSRFSGSGSGTDFTLTISSL |
| | | QPEDFATYYCQQSGSWPYTFGQGTKLEIKGGGSGGSG |
| | | GCPPCGGSGGEVQLVESGGGLVKPGGSLRLSCAASGF |
| | | TFSRYNMNWVRQAPGKGLEWVSSISTSSNYIYYADSV |
| | | KGRFTFSRDNAKNSLDLQMSGLRAEDTAIYYCTRGW |
| | | GPFDYWGCGTLVTVSS |
| 28 | CD3 spFv | DIQMTQSPSSLSASVGDRVTITCRARQSIGTAIHWYQQ |
| | | KPGCAPKLLIKYASESISGVPSRFSGSGSGTDFTLTISSL |
| | | QPEDFATYYCQQSGSWPYTFGQGTKLEIKGGGSGGSG |
| | | GCPPCGGSGGEVQLVESGGGLVKPGGSLRLSCAASGF |
| | | TFSRYNMNWVRQAPGKGLEWVSSISTSSNYIYYADSV |
| | | KGRFTFSRDNAKNSLDLQMSGLRAEDTAIYYCTRGW |
| | | GPFDYWGCGTLVTVSSEPKSSDKTHTCPPCPAPEAAG |
| | | GPSVFLFPPKPKDTLMISRTPEVTCVVVSVSHEDPEVK |
| | | FNWYVDGVEVHNAKTKPREEQYNSTYRVVSVLTVLH |
| | | QDWLNGKEYKCKVSNKALPAPIEKTISKAKGQPREPQ |
| | | VYTLPPSREEMTKNQVSLWCLVKGFYPSDIAVEWESN |
| | | GQPENNYKTTPPVLDSDGSFFLYSKLTVDKSRWQQG |
| | | NVFSCSVMHEALHNHYTQKSLSLSPGK |

### B. Immunogenicity Risk Assessment

CALRmut is a neoantigen present in malignant MPN clonal cells but not found in normal cells. Therefore, C3CRB73, with its CALRmut-targeting arm, is not expected to bind or impact normal cells. In vivo animal models were not considered relevant for nonclinical safety testing. This is due to an absence of the CALRmut target in nonclinical species (ie, mice, rats, dogs, NHPs), the lack of cross reactivity to wild-type CALR, and absence of the CD3 binder's (CD3 W245) cross-reactivity in nonclinical species including NHPs. For this reason, the nonclinical safety risk assessment is based primarily on in silico, in vitro, and ex vivo data. Specifically, the nonclinical safety assessment consists of data confirming the specificity of C3CRB73 and the absence of CALRmut in normal human and NHP tissues as well as an evaluation of the potential effect of soluble CALRmut on the binding, T-cell activation, and cytotoxicity of C3CRB73.

Studies conducted to assess the specificity of C3CRB73 included a human protein screening array (Retrogenix Assay), a Good Laboratory Practices (GLP)-compliant Tissue Cross-Reactivity Study, and in vitro-based cellular functional assays. C3CRB73 was specific to the CALRmut antigen as tested in the Retrogenix human plasma membrane array screen, the tumor-antigen-negative cellular functional assay, and in the GLP-compliant tissue cross-reactivity study. Importantly, C3CRB73 did not bind to CALR wild-type protein.

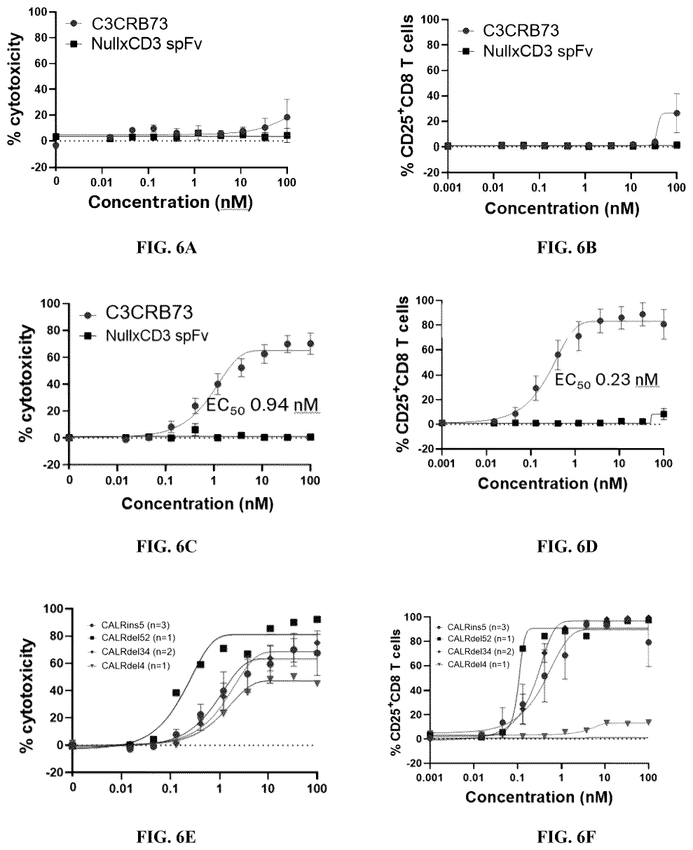

In vitro, C3CRB73-mediated CALRmut-selective T-cell mediated cytotoxicity to CALRmut-engineered cell lines or CALRmut patient-derived CD34+ cells (FIGS. 6C-6F). No cytotoxicity was observed in cells without CALRmut (FIGS. 6A and 6B). T-cell activation only occurred in the presence of CALRmut-positive tumor cells. The presence of soluble CALRmut, using either CALRmut patient-derived or recombinant CALRmut protein, did not affect C3CRB73 binding or T-cell-mediated cytotoxicity to cancer cells expressing MPL and CALRmut or the MPL alone. No evidence of off-target binding of C3CRB73 in the presence of soluble CALRmut was found in several other target-negative cells tested.

The antibody sequences were analyzed for potential immunogenicity using the T-regulatory (Treg) adjusted scores from the EpiVax EpiMatrix in silico immunogenicity prediction program (Table 2). The EpiVax program computationally calculates the binding potential to the most common HLA molecules within each of the “supertypes”. The report provides results that are representative of >90% of human populations worldwide without the necessity of testing each haplotype individually. The EpiVax score is calculated by aggregating the EpiMatrix scores of all predicted T-cell epitopes contained within a given protein sequence and adjusting for expected T-cell epitope content and protein length. Although based on extensive in silico analysis, the EpiVax EpiMatrix score has limited experimental ability to predict immunogenicity in patients (Table 3). Results in Table 2 indicate that both the anti-CD3 arm v-region and the anti-CALRmut v-region have acceptable scores. Thus, C3CRB73 is not considered to present a significant risk for immunogenicity.

TABLE 2
Immunogenicity assessment

| Description | EpiVax Score |
|---|---|
| CD3 spFv VL | −2.39 |
| CD3 spFv VH | −42.70 |
| CALRmut VL | −34.58 |
| CALRmut VH | −10.91 |

TABLE 3
EpiVax score criteria and interpretation
for potential immunogenicity

| EpiVax Score | Immunogenicity Criteria |
|---|---|
| <−20 | Ideal |
| −20 to +20 | Acceptable |
| >+20 | At risk |

### Example 2: In Vivo Pharmacology and Immunogenicity Studies of CALRmut x CD3 Bispecific Antibody

### A. PK Study of C3CRB73 in Female Cynomolgus Monkeys

The PK of C3CRB73 was studied in female cynomolgus monkeys. Three animals (n=3) were administered a single dose of 0.5 mg/kg C3CRB73 IV. Blood samples were collected for 30 days post-dose. PK parameters were estimated using non-compartmental analysis. Mean Cmax was 15.63±2.02 μg/mL and mean area under the time-concentration curve (AUCinf) was 104.18±35.82 μg·day/mL.

B. C3CRB73-induced T cell-mediated efficacy of CALRmut Xenografts

The antitumor efficacy of C3CRB73 was evaluated in SC human megakaryoplastic leukemia ELF-153 MPL CALRdel52 and disseminated acute myeloid leukemia (AML) OCI-M2 MPL CALRins5 xenografts in T cell-humanized mice. For all studies, female NSG mice (Charles River Labs, Lyon, France) were used to provide a suitable host for reconstituting a human immune system using human donor CD3+ pan T cells. Mice were inoculated SC with 3×106 ELF 153 MPL CALRdel52 or intravenously (IV) with 0.5×106 OCI-M2 MPL CALRins5 tumor cells prior to T cell engraftment. The tumor implantation day was designated as Day 0 of the study.

Expanded human pan T cells were inoculated intraperitoneally (IP) into NSG mice to humanize the immune system. Humanization of mice with CD3-expressing T cells provided effector cells to facilitate C3CRB73-mediated cytotoxicity to human CALRmut-expressing tumor cells. T cell-humanized mice were given Fc block antibody and intravenous immunoglobulin (IVIg) IP at least 30 minutes prior to C3CRB73 dosing to correct for the low Ig environment in NSG mice.

In the prevention study, NSG mice were randomly assigned into groups of n=10 animals, implanted SC with ELF-153 MPL CALRdel52 cells, humanized with pan T cells on Day 3 post tumor cell implantation, and treated twice weekly for a total of 8 doses with 1, 5, or 20 mg/kg C3CRB73 IP starting on Day 4 post cell implantation.

Percent tumor growth inhibition (TGI) of SC ELF-153 MPL CALRdel52 xenografts was calculated on Day 24 post tumor implantation, when two thirds of vehicle-treated (i.e., phosphate-buffered saline [PBS]) control animals remained on study. Statistically significant TGI was observed with C3CRB73 at 1, 5, and 20 mg/kg resulting in 97.4% (p<0.0001), 99.3% (p<0.0001), and 94.9% (p<0.0001) TGI, respectively, as compared with vehicle-treated controls (FIG. 2). No signs of graft-versus-host disease (GvHD)-related morbidity due to the humanization with T cells was observed in this study.

In the efficacy study, NSG mice were randomized into groups of n=10 animals and humanized with T cells 6 days post OCI-M2 MPL CALRins5 tumor cell implantation. IP treatment with 1, 5, or 20 mg/kg C3CRB73 was initiated on Day 7 post tumor cell implantation twice weekly for a total of 10 doses. Mice were monitored daily for negative clinical signs (i.e., lethargy, ruffled and matted coat, hunched posture, hind limb paralysis, cyanotic extremities, or dyspnea) or excessive body weight loss (i.e., ≥20%) as compared to initial body weight. Animals showing negative clinical signs or excessive body weight loss, were removed from the study and humanely euthanized.

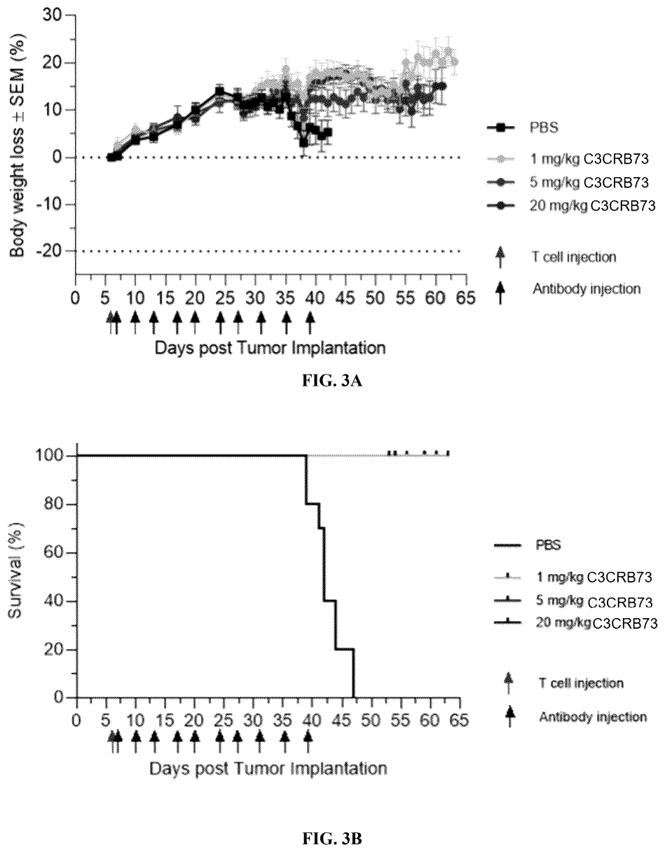

All animals in the vehicle-treated (i.e., PBS) control group were removed from study by Day 47 due to significant body weight loss and tumor burden morbidity (hind limb paralysis). Mice treated with 1, 5, or 20 mg/kg of C3CRB73 remained on study until termination of the experiment (Day 63) or were removed from study due to GvHD caused by human T cells reacting to mouse tissue antigens. Treatment with C3CRB73 significantly increased lifespan (ILS) with >50% ILS (p<0.05) for all dose levels as compared to vehicle-treated control mice (FIGS. 3A and 3B). As defined by National Cancer Institute criteria, ≥25% ILS is considered biologically significant.

Increasing levels of soluble CALRmut protein in plasma has been reported to correlate with the disease progression. In the OCI-M2 MPL CALRins5 disseminated model, limited soluble CALRmut levels (˜0.2 ng/mL) were detected in plasma of animals treated with 1, 5, and 20 mg/kg C3CRB73, while soluble CALRmut levels of 2.55 ng/mL (corresponding to 12.75 ng/mL in human setting) were measured in PBS-treated control mice. These results further confirm antitumor activity of C3CRB73.

In both studies, treatment with 1, 5, or 20 mg/kg C3CRB73 did not show signs of body weight loss or other signs of gross toxicity. First signs of GvHD-related morbidity due to the humanization with T cells was observed on Day 53.

### B. Summary and Conclusions

In vivo, C3CRB73 prevented tumor growth in the ELF-153 MPL CALRdel52 SC xenograft model and significantly increased lifespan in the OCI-M2 MPL CALRins5 disseminated xenograft model.

### Example 3: Pharmacokinetics and Pharmacodynamic Analysis

### A. Pharmacokinetics in Cynomolgus Monkeys

The pharmacokinetics (PK) properties of C3CRB73 were characterized in female cynomolgus monkeys following a single intravenous (IV) dose of C3CRB73 at 0.5 mg/kg (n=3/group) in a non-GLP study. Serum samples were collected at 0.04, 0.25, 1, 2, 4, 7, 10, 14, 18, 23, 28, and 30 days post dose. A qualified research method, Meso Scale Discovery (MSD) electro-chemiluminescent immunoassay (ECLIA) with a lower limit of quantitation (LLOQ) of 0.08 μg/mL in cynomolgus monkey serum, was used to quantitate concentrations of total C3CRB73. A biotinylated and ruthenium-labeled mouse anti-human IgG monoclonal antibody was employed for capture and detection of C3CRB73.

The serum concentration-time profile of C3CRB73 in monkey is illustrated in FIG. 5. Serum PK parameters, which were estimated using non-compartmental analysis (NCA), are shown in Table 4.

TABLE 4
PK parameters of C3CRB73 in female cynomolgus monkey
serum following a single 0.5 mg/kg IV dose.

| Dose | | | | |
|---|---|---|---|---|
| mg/kg | Cₘₐₓ | AUCₗₐₛₜ | AUC_{inf} | CL |
| (route) | (μg/mL) | (μg · day/mL) | (μg · day/mL) | (mL/day/kg) |
| 0.5 (IV) | 15.63 (2.02) | 104.18 (35.82) | 130.25 (57.47) | 4.3 (1.58) |

AUC_{inf}, area under the concentration-time curve extrapolated to infinity; AUCₗₐₛₜ, area under the concentration-time curve up to the last sampling point; CL, total clearance; Cₘₐₓ, maximum serum concentration; IV, intravenous. Values are mean with standard deviation between brackets (n = 3/group).

In addition, serum concentration-time data were fitted to a linear 2 compartment PK model for PK parameter estimation. No apparent anti-drug antibodies (ADA) was observed in any animal.

### B. Preliminary Efficacious Dose Prediction

Allometric scaling was used to predict human PK of C3CRB73 based on PK parameter estimates from monkeys assuming a body weight of 70 kg for human and 3 kg for cynomolgus monkey. Scaling factors were 0.85 for clearance-associated parameters (total clearance [CL] and distribution clearance [Q]), 1.0 for distribution-volume-associated parameters (volume of the central compartment [Vc] and volume of the peripheral compartment [Vt]). Model-estimated cynomolgus monkey parameters, as well as projected human PK parameters are listed in Table 5. The estimated T1/2 in cynomolgus monkeys is 10.9 days using a 2-compartment model and predicted systemic T1/2 of C3CRB73 in humans is approximately 17.7 days. The SC bioavailability (ie, 70%) and absorption rate constant (i.e., 0.3 L/day) in humans were assumed based on internal clinical experience with CD3 redirectors.

TABLE 5
Model-estimated parameters of C3CRB73 in cynomolgus
monkeys and projected human PK parameters

| | Monkey | Predicted |
|---|---|---|
| Parameter | estimate | human |
| (unit) | (% RSE) | parameter |
| V_{c} (mL/kg) | 31.59 (4.10) | 31.59 |
| Q (mL/day/kg) | 11.95 (26.90) | 7.37 |
| CL (mL/day/kg) | 4.16 (11.65) | 2.57 |
| Vₜ (mL/kg) | 28.81 (25.49) | 28.81 |
| F (%) | — | 70 (assumed) |
| kₐ (1/day) | — | 0.3 (assumed) |

CL, total clearance; F, bioavailability; kₐ, absorption rate constant; PK, pharmacokinetics; Q, distribution clearance; RSE, relative standard error; V_{c}, volume of the central compartment; Vₜ, volume of the peripheral compartment.

The preliminary dose projections are based on the following 3 main assumptions.

The first assumption is that human IV PK is predictable using allometric scaling based on body weight from IV bolus data in cynomolgus monkey.

Secondly, since neither the anti-CALRmut arm nor the anti-CD3 arm of C3CRB73 was cross-reactive with cynomolgus monkey, target-mediated drug disposition (TMDD) was not incorporated in the current PK model. However, TMDD might be anticipated in humans, especially at low dose levels.

Finally, in vitro T cell activation, cytotoxicity, and cytokine release in ELF 153 MPL CALRmut cells at an E:T ratio of 1:1 in patient whole blood is considered a relevant system to CALRmut-positive tumors that was used for FIH starting dose prediction. The E:T ratio data used for dose predictions is physiologically relevant.

C. Preliminary Recommended range for human Phase 1 Trial

A MABEL-based approach was used to provide a preliminary estimate of the FIH starting dose of C3CRB73. The preliminary MABEL concentration of 0.64 nM was determined from the median EC50 value of the ELF-153 MPL CALRins5 cells cytotoxicity using patient whole blood, the most sensitive and relevant assay based on the current data sets (see U.S. Provisional Application No. 63/516,520). A single SC administration at 0.6 mg flat dose (assuming 70 kg mean human body weight) is predicted to result in a maximum serum concentration (Cmax) of C3CRB73 right below the median EC50 value of the most sensitive in vitro assay.

### Example 4: A First-in-Human Study of the Safety, Pharmacokinetics, and Pharmacodynamics of a CALRmutxCD3 Bispecific Antibody for CALR-mutated Myeloproliferative Neoplasms

### A. Protocol Summary

The clinical protocol is an open-label, Phase 1 study to evaluate the safety, pharmacokinetics (PK), pharmacodynamics (PD), and preliminary clinical activity of COMPOUND 1, a clinical batch of C3CRB73, a T-cell redirecting bispecific antibody for CALR-mutated myeloproliferative neoplasms, e.g., with adult participants with CALRmut MPNs diagnosed as either essential thrombocythemia (ET) or myelofibrosis (MF).

### B. Objectives and Endpoints

Below is the list of objectives and endpoints that are evaluated in this study, delineating the details in alignment with the general objectives listed.

TABLE 6
Objectives and Endpoints

| | |
|---|---|
| Objectives | Endpoints |

Primary

| | |
|---|---|
| Part 1 (Dose Escalation) | Frequency and severity of AEs (including DLTs) |
| To characterize safety and to determine the | |
| putative RP2D(s) and optimal dosing | |
| schedule(s) of COMPOUND 1 | |
| Part 2 (Cohort Expansion) | Frequency and severity of AEs |
| To further characterize the safety of | |
| COMPOUND 1 at the putative RP2D(s) | |

Secondary

| | |
|---|---|
| To characterize the PK of COMPOUND 1 | Serum COMPOUND 1 concentrations and estimation of PK |
| | parameters for |
| | COMPOUND 1 including but not limited to AUC_{τ}, Cₘₐₓ, |
| | and Cₘᵢₙ |
| To assess the immunogenicity of | Presence of anti-COMPOUND 1 antibodies |
| COMPOUND 1 | |
| Evaluate the preliminary clinical activity of | Overall response rate (CR + PR), CR rate, time to response, and |
| COMPOUND 1 in participants with ET or MF | DOR per investigator assessment. Response assessed according |
| who have become therapy intolerant or | to: |
| resistant | Modified Response criteria for ET and polycythemia |
| | vera: result of European LeukemiaNet consensus |
| | conference (Barosi 2009, Blood. 113: 4829-4833) |
| | Modified response criteria for myelofibrosis: |
| | International Working Group-Myeloproliferative |
| | Neoplasm Research and Treatment (IWG-MRT) and |
| | European LeukemiaNet consensus report (Tefferi 2013, |
| | Blood. 122; 1395-1398) |
| | (Part 2 Only): Patient Reported Outcome |
| | measure of symptom burden of disease during |
| | Cohort Expansion |

Exploratory
Explore biomarkers of response to COMPOUND 1 which may include, but are not limited to, mutation and
expression profile associated with response/resistance, cytokine profile, and immune cell subsets.
Assessment of cytokine profile and T-cell margination, and characterization to demonstrate therapeutic
engagement and associations with MOA and MOR.
IHC/IF to confirm T-cell distribution in relation to megakaryocytes in bone marrow.
Explore the relationships between PK, PD (soluble CALRmut), RO, biomarkers, AE profiles, and clinical
activity or resistance to COMPOUND 1.
Abbreviations: AE = adverse event; AUC_{τ}.= area under the serum concentration vs. time curve during a dosing interval (τ) at steady state; Cₘₐₓ = maximum serum drug concentration; Cₘᵢₙ = minimum serum drug concentration; CR = complete remission; DLT = dose-limiting toxicity; DOR = duration of response; ET = essential thrombocythemia; IF = immunofluorescence; IHC = immunohistochemistry; MF = myelofibrosis; MOA = mechanism of action; MOR = mechanism of resistance; PD = pharmacodynamic; PK = pharmacokinetic; PR = partial remission; RO = receptor occupancy; RP2D = recommended Phase 2 dose.

### C. Overall Design

The study will be conducted in 2 parts: Part 1 (Dose Escalation) followed by Part 2 (Cohort Expansion) (FIG. 8). Part 1 is designed to determine putative RP2D(s) and optimal dosing schedule(s) based on safety, PK, PD, and preliminary assessment of efficacy across several dose regimens. The putative RP2D(s) and optimal dosing schedule(s) determined in Part 1 will be further studied in Part 2 in participants with specific presentations and risk profiles of CALRmut MPN.

The study treatment will be administered subcutaneously (SC) at a dose(s) assigned by the sponsor according to the dose escalation or cohort expansion strategy outlined below and in FIG. 8. Study treatment will be administered as an outpatient but with a hospital stay of a minimum of 72 hours for observation of early inflammatory adverse effects after each step-up dose and the first treatment dose of COMPOUND 1.

In Part 1, it is anticipated that 40 to 60 participants will be enrolled in separate disease specific cohorts (e.g., CALRmut MF and ET), and the total number of participants will depend on the number of dose levels explored to identify the putative RP2D(s). Initial enrollment efforts will focus on CALRmut MF (Int-2 and high risk) only, with consideration to enroll CALRmut MF (Int-1) and eventually CALRmut ET participants upon review of preliminary safety data with SET endorsement. In Part 2, approximately 20 participants will be enrolled for each candidate RP2D disease specific expansion cohort. The total number of participants enrolled in the clinical trial is anticipated to be approximately 100 participants (DLT evaluable).

A Study Evaluation Team (SET) will be commissioned for this study. During the study, safety will be monitored by the SET at each dose escalation step and at regular intervals during Part 2. Cumulative data from subsequent treatment cycles will also be monitored for late-onset toxicities, including prolonged cytopenias. SET decisions will be based on the review of all available data including, but not limited to safety data, PK, PD, and preliminary antitumor activity. The SET may propose dose escalation, dose modifications, changes to the treatment and procedure schedule(s), or modifications in study conduct. For example, target dose escalation levels, starting at 0.6 mg subcutaneous injection, will no more than double between dose escalations, as described in Table 7.

TABLE 7
Maximum Target Dose Escalations

| Target Dose Series | Maximum Dose |
|---|---|
| 1 | 0.6 mg |
| 2 | 1.2 mg |
| 3 | 2.4 mg |
| 4 | 4.8 mg |
| 5 | 9.6 mg |
| 6 | 19.2 mg |
| 7 | 38.4 mg |
| 8 | 76.8 mg |

### Part 1

Part 1 (Dose Escalation) is designed to determine putative RP2D(s) and optimal dosing schedule(s) for further study in Part 2 for COMPOUND 1. The overall study population will comprise participants with CALRmut ET or MF into separate dose escalation cohorts who have exhausted therapeutic options. However, initial enrollment efforts will focus on CALRmut Intermediate-2 (Int-2) and High-risk MF participants only. Once preliminary safety data is available in this CALRmut MF population (including frequency and severity of CRS, coagulopathy, and myelosuppression), with SET review and endorsement of favorable risk/benefit profile, inclusion of Intermediate-1 (Int-1) MF patients who have failed prior JAKi therapy may be considered for enrollment.

The study will explore successively higher doses, beginning at the MABEL-based starting dose of COMPOUND 1 initially in CALRmut MF participants only aligned with the approach described above, across successive, respective cohorts. Enrollment of mutCALR ET participants will be considered only after review of available clinical data for at least 3 MF cohorts and a minimum of 12 MF participants with SET endorsement to inform a safe dose escalation approach in ET participants. The initial starting dose for ET participants will not exceed 2.4 mg.

The assessment of safety, efficacy, PK, and PD will be reviewed by the SET and the sponsor, who will agree before any escalation of the dose. The assessment of safety for a given cohort will include review and discussion of the AEs for the participants in that cohort. Safety for a cohort will also be assessed by the proportion of participants in the cohort that experience a DLT.

In Part 1, the SET and sponsor will follow the Bayesian Optimal Interval (BOIN) design, which will guide against escalation of the dose if the proportion of participants in the DLT Evaluable Analysis Set who experience a DLT in the current cohort is >25%.

Part 1 will explore the initial starting dose of 0.6 mg SC administered every 3 weeks. With dose escalation beginning at the MABEL-based starting dose, dose escalation decisions will be guided by the BOIN design with SET endorsement.

One or more putative RP2D(s) and optimal dosing schedule(s) may be determined by pooling and evaluating all available PK, PD, target engagement, efficacy, safety, and tolerability data across the disease-specific cohorts and studied further in additional participants in Part 2 (Cohort Expansion) upon agreement of the SET and sponsor. The DLT evaluation period begins with administration of the first dose of COMPOUND 1 (either step-up or target dose) and encompasses the period before administration of the scheduled 2nd administration of target dose. The DLT evaluation periods for various dosing scenarios that may be explored in Part 1 are described in the table below. Any participant not completing the DLT evaluation period for reasons other than toxicity will be considered non-evaluable for DLTs and may be replaced for DLT evaluation. A DLT is any adverse event attributed to study treatment that meets the criteria for severity and duration in Table 9, and that occurs during the evaluation periods summarized in Table 8 unless it can be incontrovertibly attributed to disease or other extraneous cause such as an accident.

TABLE 8
DLT Evaluation Periods for Q3W Scheduling as Required

| | | | | | | | Projected DLT |
|---|---|---|---|---|---|---|---|
| | Dose Day Schedule | | | | | | Evaluation |
| | Day 1 | Day 8 | Day 15 | Day 22 | Day 29 | Day 36 | Period (days) |
| Q3W | Target | | | Target | | | 21 |
| | Dose #1 | | | Dose #2 | | | |
| Q3W-SU1 | Step-up | Target | | | Target | | 28 |
| | #1 | Dose #1 | | | Dose #2 | | |
| Q3W-SU2 | Step-up | Step-up | Target | | | Target | 35 |
| | #1 | #2 | Dose #1 | | | Dose #2 | |

Abbreviations: DLT = dose-limiting toxicity; Q2W = once every 2 weeks; Q3W = once every 3 weeks; SU = step-up.

TABLE 9
Dose-Limiting Toxicity Criteria

General
Any toxicity that would require discontinuation of treatment
Any Grade 5 toxicity

| Criteria for Non-hematological Toxicity | Exceptions |
|---|---|
| Grade 3 or 4 laboratory abnormalities | Grade 3 lasting ≤5 days or Grade 4 lasting <24 hours |
| | if not associated with clinical complications |
| | unless the abnormality is part of a syndrome (eg, CRS), |
| | in which case the grade of the syndrome should dictate |
| | the DLT assessment (see also specific chemistries below) |
| Grade 3 or 4 Tumor Lysis Syndrome | Recovers in ≤5 days |
| Hy's Law with modification | Hy's law modification for participants with baseline AST or |
| ALT or AST value >3x ULN, and total | ALT > ULN, eg, due to liver extra-medullary disease, ALT or |
| bilirubin >2x ULN, and ALP ≤2x ULN, with no | AST >3x baseline or AST or ALT >8x ULN, whichever is |
| alternative etiology. | lower, combined with total bilirubin >2x baseline AND >2x |
| | ULN, with no alternative etiology, will be considered as |
| | meeting modified Hy's law. |
| Any other Grade 3 | First occurrence of CRS or sARR that resolves to |
| | Grade ≤2 within 72 hours |
| | Asthenia, anorexia, fever, constipation, nausea, vomiting, |
| | or diarrhea lasting 72 hous. |
| Any other Grade 4 | None |

| Criteria for Hematological Toxicity | Exceptions |
|---|---|
| ET | |
| Neutrophil count decreased Grade 4 >7 days | |
| despite supportive care according to | |
| institutional standards. | |
| MF | |
| Neutrophil count decreased Grade 4 >14 days | |
| despite supportive care according to | |
| institutional standards | |
| Grade 4 platelet count decrease for >7 days, or | Transfusions given for Grade 3 thrombocytopenia without |
| Grade ≥3 with Grade ≥2 bleeding | clinically significant bleeding will be reviewed by the SET to |
| | determine relevance as a DLT |
| Grade 4 anemia that is unexplained by | |
| underlying disease. | |

Abbreviations: ALP = alkaline phosphatase; ALT = alanine aminotransferase; AST = aspartate aminotransferase; CRS = cytokine release syndrome; DLT = dose-limiting toxicity; sARR = systemic administration-related reaction; ULN = upper limit of normal.

### Part 2

The objective of a cohort in Part 2 is to characterize further the safety, as well as to evaluate preliminarily the efficacy of a putative RP2D and dosing schedule in a particular disease category. 1 or more putative RP2D(s) and optimal dosing schedule(s) may be selected for Part 2.

Respective cohorts of approximately 20 participants each may be enrolled for participants with high-risk cases of ET or MF who are resistant to or intolerant of existing therapies at each dose and schedule.

Based on emerging data, disease categories may be added, excluded, or revised to other histologies, as determined by the SET. The objective of a cohort in Part 2 is to further characterize the safety as well as preliminarily evaluate the efficacy of a putative RP2D and optimal dosing schedule in a particular disease category.

### Step-Up Dose

Step-up dose strategies have previously been effectively utilized for T-cell redirecting bispecific antibodies to mitigate acute cytokine-mediated toxicities associated with first dose administration.

In this study, step-up dose(s) may be explored to mitigate the occurrence of cytokine-mediated toxicities based on evaluation of the totality of clinical and supportive data as outlined by the SET. Step-up dosing will be guided by the experience of earlier dosing cohorts that have cleared their DLT periods. Given the SC route of administration and anticipated therapeutic range for this molecule, initiation of step-up dosing may precede the clinical observance of these types of AEs. Step-up will involve administration of 1 or more doses prior to a subsequent target dose. The step-up dose(s) will be less than or equal to a dose that has been previously safe cleared by the SET upon review of emerging safety data (e.g., DLTs) supporting continued dose escalation through application of the BOIN design.

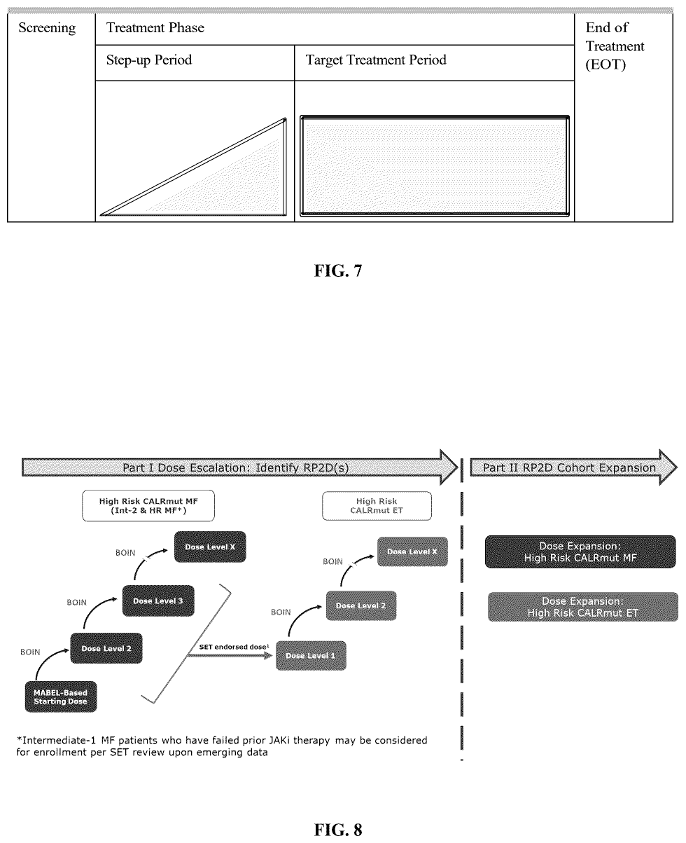

FIG. 7 illustrates how to incorporate a step-up dosing schedule in the Schedule of Activities (SoA) if 1 or more step-up doses are administered.

### Study Interventions

The study will be initiated with flat dosing and SC injection as route of administration. Dose escalation will be initiated at a starting dose of 0.6 mg SC administered every 21 days (Q3W), and will be guided by BOIN design and follow dose increment rules. Other dose schedules (e.g., once weekly, Q2W, Q4W, with or without one or more step-up dose) can be evaluated based on emerging safety and PK data after approval by the SET.

Multiple SC injections may be administered within the administration visit. Doses will be administered at alternating locations on the abdomen (other locations may be acceptable based on treating physician/sponsor's medical monitor discretion). Maximum volume to be administered for each SC injection site will be 2 mL. In case of significant discomfort during manual injection, the total dose may be given in multiple separate locations.

The observation period begins after the SC injection. All participants must be hospitalized for a minimum of 72 hours after the end of administration of each step-up dose and first treatment dose of COMPOUND 1. Participants should be observed for at least 6 hours after each study treatment administration, for the first 4 target doses (the SET may review and increase or decrease the observation period as required).

### D. Study Population

Screening for eligible participants will be performed within 30 days before administration of the study treatment. The inclusion and exclusion criteria for enrolling participants in this study are described below.

### Inclusion Criteria

Each potential participant must satisfy all of the following criteria to be enrolled in the study:
1. Be ≥18 years of age (or the legal age of majority in the jurisdiction in which the study is taking place, whichever the greater) at the time of informed consent.
2. Have a diagnosis of either ET or MF as defined by the 2022 WHO criteria (WHO Classification of Tumours Editorial Board 2022) that meets the stated risk criteria:

### Essential Thrombocythemia

High-risk of thrombosis or hemorrhage, defined as any 1 of the following:
   Age >60 years
   Platelet count >1500×10⁹/L at any point during the participant's disease
   Previous documented thrombosis (including transient ischemic attack [TIA]), erythromelalgia, or migraine (severe, recurrent, requiring medications, and felt to be secondary to the MPN) either after diagnosis or within 10 years before diagnosis and considered to be disease-related.
   Previous hemorrhage or coagulopathy related to ET
   Diabetes mellitus or hypertension requiring pharmacological therapy >6 months AND
Intolerant or resistant or refractory to hydroxyurea (HU), defined as any 1 of the following according to NCCN Guidelines Version 1.2023:
   Platelet count >600×10⁹/L after 3 months of at least 2 g/day or maximum tolerated dose (MTD) of HU (2.5 g/day in participants with a body weight >80 kg)
   Platelet count >400×10⁹/L and WBC <2.5×10⁹/L at any dose of HU (for a period of at least 3 months)
   Platelet count >400×10⁹/L and hemoglobulin <10 g/dL at any dose of HU (for a period of at least 3 months)
   Presence of leg ulcers or other unacceptable mucocutaneous manifestations at any dose of HU
   HU-related fever
      Myelofibrosis (primary or post-ET)

### Primary Myelofibrosis

Dynamic International Prognostic Scoring System (DIPSS; Passamonti 2010, Blood. 115:1703-1708) Intermediate 2 or High-Risk with a blast percentage not consistently exceeding 20% in blood or bone marrow. SET to consider enrollment of Intermediate-1 based on emergent data.

### Post-ET MF

MYSEC-PM (Passamonti 2017, Leukemia. 31:2726-2731) Intermediate 2 or High-Risk with a blast percentage not consistently exceeding 20% in blood or bone marrow. SET to consider enrollment of Intermediate-1 based on emergent data.
And
Ineligible or intolerant or resistant/refractory to JAK inhibitor (JAKi) therapy.
   Ineligible
   JAKi contraindicated due to prior history of severe infections such as tuberculosis, progressive multifocal leukoencephalopathy, and skin malignancies that are known to be associated or exacerbated by JAKi, or other significant considerations as documented by the treating physician.
   Intolerant
   a) Hematologic toxicity—platelet count <50×10⁹/L and/or neutrophils ≤0.5×10⁹/L despite recommended dose adjustments and interruptions; or
   b) ≥Grade 3 non-hematologic toxicity as per the Common Terminology Criteria for Adverse Events (CTCAE) Version 5.0, 2017
   Resistant Refractory
   Evidence would include:
      a) persistent splenomegaly, or
      b) lack of symptom improvement, or
      c) persistent leukoerythroblastosis, or
      d) anemia <10 g/dL, or
      e) leukocytosis >11×10⁹/L
3. Positive for a CALR driver mutation of ET or MF.
4. Have received prior therapy(ies):
   Essential Thrombocythemia
   At least 2 lines (unless unavailable or contraindicated) of prior cytoreductive therapy, at least 1 of which must have been HU.
   Myelofibrosis
   At least 1 prior JAKi therapy unless ineligible as described in Criterion 2.
5. Have discontinued concurrent use of the following therapies:
   Essential Thrombocythemia
   Interferon-α (pegylated or standard preparation), anagrelide, busulfan.
   Exception: HU is permitted.
   Mvelofibrosis
   JAKi, immunomodulatory drug therapy (such as thalidomide), danazol, or other therapy intended to lead to disease modification.
6. Have an ECOG performance status grade of ≤2 (Oken 1982, Am J Clin Oncol. 5:649-655)
7. Have the following clinical hematology laboratory values predose:
   a. Hemoglobin ≥8.0 g/dL
   b. Neutrophils ≥0.75×10⁹/L without the assistance of granulocyte growth factors within 4 weeks of the first dose of study drug
   c. Platelet counts between 50×10⁹/L and 600×10⁹/L without the assistance of thrombopoietic factors or transfusions
8. Participants should have the following clinical chemistry laboratory values predose:
   a. ALT: ≤3×ULN
   b. AST: ≤3×ULN
   c. Direct bilirubin: ≤1.5×ULN
   d. Renal function: Estimated or measured glomerular filtration rate ≥40 mL/min per CKD-EPI formula
9. Known HIV-positive participants are eligible if they meet all of the following:
   a. No detectable viral load (ie, <50 copies/mL) at screening
   b. CD4+ count >300 cells/mm3 at screening
   c. No AIDS-defining opportunistic infection within 6 months of screening
   d. Receiving highly active antiretroviral therapy (HAART). Any changes in HAART due to resistance/progression should occur at least 3 months prior to screening. A change in HAART due to toxicity is allowed up to 4 weeks prior to screening.
   Note: HAART that could interfere with study treatment is excluded (consult the sponsor for a review of medications prior to enrollment).
10. A participant of childbearing potential must have a negative highly sensitive serum (eg, beta-human chorionic gonadotropin [0-hCG]) pregnancy test at screening and within 72 hours of the first dose of study treatment and must agree to further serum or urine pregnancy tests during the study.
11. A participant of childbearing potential must practice at least 1 highly effective method of contraception throughout the study and at least 90 days after the last dose of study treatment.
   Note: If the participant becomes of childbearing potential after the start of the study, the participant must comply with this criterion.
12. A participant using oral contraceptives must use an additional barrier contraceptive method.
13. A participant must agree not to be pregnant, breastfeeding, or planning to become pregnant while enrolled in this study or within 90 days after the last dose of study treatment.
14. A participant must agree not to donate gametes (ie, eggs or sperm) or freeze for future use for the purposes of assisted reproduction during the study and for a period of 6 months after receiving the last dose of study treatment. Participants should consider preservation of gametes prior to study treatment as anticancer treatments may impair fertility.
15. A participant must wear a condom when engaging in any activity that allows for passage of ejaculate to another person during the study and for at least 90 days after receiving the last dose of study treatment.
If a participant's partner is of childbearing potential, the participant must use condoms (with or without spermicide) and the partner must also be practicing a highly effective method of contraception. A vasectomized male participant must still use a condom (with or without spermicide), but the partner is not required to use contraception.
16. A participant must agree not to plan to father a child while enrolled in this study or within 3 months after the last dose of study treatment.
17. Must sign an ICF indicating that the participant understands the purpose of, and procedures required for, the study and is willing to participate in the study.
18. Be willing and able to adhere to the lifestyle restrictions specified in this protocol.

### Exclusion Criteria

Any potential participant who meets any of the following criteria will be excluded from participating in the study:
1. Known allergies, hypersensitivity, or intolerance to the excipients of the study treatment.
2. Chemotherapy, cytoreductive therapy, targeted therapy, or immunotherapy at 5 half-lives or 2 weeks, whichever is shorter, prior to the planned first dose of study treatment. EXCEPTIONS:
3. Any prior treatment with CALRmut-targeted therapy. Exception: participants with prior exposure to CALRmut-targeted therapy may be considered for enrollment with Sponsor approval.
4. Concurrent or recently diagnosed or treated malignancies present at the time of participant screening. Exceptions are squamous and basal cell carcinoma of the skin, carcinoma in situ of the cervix, and any malignancy that is considered cured or has minimal risk of recurrence within 1 year of first dose of study treatment in the opinion of both the investigator and sponsor's medical monitor. Participants cured of another malignant disease with no sign of relapse ≥3 years after treatment ended are allowed to enter the study.
5. Prior solid organ transplantation.
6. Either of the following regarding hematopoietic stem cell transplantation:
   a. Prior treatment with allogenic stem cell transplant ≤6 months before the first dose of COMPOUND 1 or
   b. Evidence of graft versus host disease (GVHD) that requires immunosuppressant therapy
7. Active autoimmune disease that requires systemic immunosuppressive medications (eg, chronic corticosteroid, methotrexate, or tacrolimus).
8. Toxicities from previous anticancer therapies that have not resolved to baseline levels, or to Grade 1 or less, or to Grade ≤2 for alopecia, peripheral neuropathy, and vitiligo.
9. History of clinically significant cardiovascular disease within 6 months prior to the first dose of study treatment including, but not limited to:
   a. Myocardial infarction
   b. Severe or unstable angina
   c. Clinically significant ventricular arrhythmias or unexplained syncope, not believed to be vasovagal in nature or due to dehydration
   d. History of severe non-ischemic cardiomyopathy
   e. Congestive heart failure (New York Heart Association class III-IV)
   f Uncontrolled (persistent) hypertension: systolic blood pressure >159 mm Hg OR diastolic blood pressure >99 mm Hg
   g. Stroke or transient ischemic attack
   h. Pericarditis or clinically significant pericardial effusion
   i. Myocarditis
   j. Endocarditis
   k. Acute ischemic limb
10. Clinically significant pulmonary compromise, particularly the need for supplemental oxygen use to maintain adequate oxygenation.
11. Evidence of active viral (including chronic Epstein-Barr virus), bacterial, or uncontrolled systemic fungal infection requiring systemic treatment within 7 days before the first dose of study treatment.
12. Fever (body temperature ≥38.0° C./100.4° F.) in the 48 hours prior to first dose of study treatment.
13. Trauma or major surgery (e.g., requiring general anesthesia) within 28 days prior to the first dose of study treatment. Note: Participants with planned surgical procedures to be conducted under local anesthesia may participate.
14. Any serious underlying medical or psychiatric condition (eg, alcohol or drug abuse), dementia or altered mental status; or any issue that would impair the ability of the participant to receive or tolerate the planned treatment at the investigational site, to understand informed consent, or that in the opinion of the investigator would contraindicate the participation in the study or confound the protocol-specified assessments or results of the study.
15. A prohibited medication that cannot be discontinued or substituted, or temporally interrupted during the study.
16. Vaccination with a live, attenuated vaccine within 4 weeks before the first administration of study treatment.
17. Body weight is <40 kg at screening and/or at the time of their first administration of study treatment.
18. Active infective hepatitis:
   Seropositive for hepatitis B: defined by a positive test for hepatitis B surface antigen (HBsAg). Participants with resolved infection (ie, participants who are HbsAg negative with antibodies to total hepatitis B core antigen [anti-HBc] with or without the presence of hepatitis B surface antibody [anti-HBs]) must be screened using real-time polymerase chain reaction (RT-PCR) measurement of hepatitis B virus (HBV) DNA levels. Those who are RT-PCR positive will be excluded. Participants with serologic findings suggestive of HBV vaccination (anti-HBs positivity as the only serologic marker) AND a known history of prior HBV vaccination, do not need to be tested for HBV DNA by RT-PCR.
   Known hepatitis C infection or positive serologic testing for hepatitis C virus (anti-HCV antibody).
   Positive hepatitis C antibody test result at screening or within 3 months prior to starting study treatment. NOTE: Participants with positive hepatitis C antibody due to prior resolved disease can be enrolled only if a confirmatory negative hepatitis C RNA test is obtained. Positive hepatitis C RNA test result at screening or within 3 months prior to first dose of study treatment. NOTE: Test is optional and participants with negative hepatitis C antibody test are not required to also undergo hepatitis C RNA testing.
   Other known clinically active liver disease of infectious origin.
19. History of Hemophagocytic Lymphohistiocytosis (HLH)/Macrophage Activation Syndrome (MAS).
20. History of pneumonitis or interstitial lung disease.
21. Those without evidence of stable anticoagulant therapy, defined as ≥4 weeks of unmodified anticoagulant therapy prior to the first administration of study treatment.

### E. Concomitant Therapy

Prior to the first dose of study treatment, corticosteroid, antihistamine, and antipyretic pre-medications will be administered to minimize the risks associated with CRS and sARR. The premedication dose(s) or schedule(s) may be reduced or omitted for subsequent doses based on emerging data after the sponsor or SET review of available data. For participants who experience a Grade 2 or higher CRS, sARR, or neurotoxicity, pretreatment corticosteroid will be required for at least 1 subsequent dose administered to that participant.

Participants in this study should receive pretreatment medication as noted below (9). Pretreatment medications may be changed based on emerging safety and other data as determined by the SET in Part 1 or 2.

TABLE 10
Pretreatment Medications

| | | | |
|---|---|---|---|
| Premedication | Dose | Administration | Required/Optional |

Glucocorticoid Medication
Note: Administer full (16 mg) dose of dexamethasone (or equivalent) noted below for first full treatment dose and,
prior to associated step-up dose(s). If no reactions are observed after the first treatment dose, then no further
glucocorticoids are required; however, an optional 8 mg of dexamethasone may be administered at the discretion
of the investigator for the second treatment dose.
If a Grade ≥2 CRS or sARR occurs, administer full (16 mg) dose of dexamethasone (or equivalent) prior to the next
study treatment administration. If no reactions are observed then administer half (i.e., 8 mg) the glucocorticoid
dose for following study treatment administration. Glucocorticoids may be omitted if no further CRS event occurs
after 2 consecutive study treatment administrations.
All medications to be dosed one hour ±30 minutes prior to CALRmut.

| | | | |
|---|---|---|---|
| Glucocorticoid | dexamethasone | IV or Oral - administer prior to | Required prior to all step-up doses and |
| | (16 mg) | study treatment administration | first treatment dose^{a} |
| Glucocorticoid | dexamethasone | IV or Oral - administer prior to | Optional - see above |
| | (8 mg) | study treatment administration | |

Other Medications

| | | | |
|---|---|---|---|
| Antihistamine | diphenhydramine | IV or Oral - administer prior to | Required prior to all step-up doses and |
| | (50 mg) or | study treatment | first treatment dose^{a}, |
| | equivalent | | Beyond these timepoints it is optional |
| Antipyretic | acetaminophen | IV or Oral - administer prior to | Required |
| | (paracetamol) | study treatment | prior to all step-up doses and first |
| | dosing per | | treatment dose^{a}, |
| | institutional | | Beyond these timepoints it is optional |
| | practice | | |
| Antiemetic | ondansetron | IV or Oral - administer prior to | Optional |
| | (16 mg) or | study treatment | |
| | equivalent | | |
| Second | loratadine or | Oral - administer day after first | Optional |
| generation | cetirizine | step-up dose and daily through | |
| antihistamine | (10 mg) or | 7 days after the first treatment | |
| (H₁ antagonist) | equivalent | dose then as needed. | |
| H₂ antagonist | ranitidine per | IV or Oral - administer prior to | Optional |
| | institutional | study treatment | |
| | practice | Oral - administer day after first | |
| | | step-up dose and daily through | |
| | | 7 days after the first treatment | |
| | | dose then as needed. | |
| Leukotriene | montelukast | Oral - administer prior to study | Optional |
| inhibitor | (10 mg) or | treatment and then daily | |
| | equivalent | through 7 days after the first | |
| | | treatment dose then as needed. | |

Abbreviations: CRS = cytokine release syndrome; IV = intravenous; sARR = systemic administration-related reaction.
^{a}Pretreatment medications are only required up to and including the first treatment dose and the step-up dose(s), if administered.

### F. Study Assessments

### Overview

Each of the 2 parts of this study is divided into 3 periods: screening, treatment, and post-treatment follow-up. All planned assessments, including clinical laboratory tests, must be completed and the results reviewed at each site visit. If multiple assessments are scheduled for the same timepoint, it is recommended that procedures be performed in the following sequence: ECG, vital signs, blood draw.

Treatment decisions will be based on safety and disease assessments performed at the local laboratory. More frequent study visits may be performed, and clinical evaluations may be repeated more frequently, if clinically indicated. Specimen collections for PK, PD, and biomarker assessments should be kept as close to the specified time as possible. Other measurements may be done earlier than specified timepoints, if needed. Actual dates and times of assessments will be recorded in the source documentation and eCRF or laboratory requisition form. Repeat or unscheduled samples (i.e., PK, PD, biomarkers) may be taken for safety reasons or for technical issues with the samples. Additional serum or urine pregnancy tests may be performed, as determined necessary by the investigator, or required by local regulation, to establish the absence of pregnancy at any time during the participation in the study.

All PRO assessments, in Part 2 only, must be conducted/completed before any tests, procedures, or other consultations to prevent influencing participant responses. Refer to the PRO completion guidelines for instructions on the administration of PROs. Symptom burden will be assessed regularly by the MPN-SAF-TSS in all participants in Part 2 and, at the Sponsor's discretion, selected participants in Part 1.

### Screening Phase

All participants must sign an ICF prior to the conduct of any study-related procedures. The screening phase begins when the first screening assessment is performed and ≤30 days before the first dose of the study treatment. If an assessment were performed as part of the participant's routine clinical evaluation and not specifically for the study, then this does not need to be repeated after signed, informed consent has been obtained provided that the assessments fulfill the study requirements and are performed within the specified timeframe prior to the first dose of study treatment. A bone marrow biopsy sample is required at screening to establish a baseline histological status of the underlying MPN, but not to confirm enrollment to the study.

A complete transfusion history for the 12 weeks prior to enrollment will be taken during the Screening Phase to include the date, type (eg, whole blood, platelets, packed cells), number of units of the transfusion as well as the hemoglobin concentration or platelet count at the time of the transfusion.

The presence of a diagnostic CALRmut genetic test is adequate for enrollment to the trial. The results of molecular testing during screening will not need to be reviewed prior to enrollment but will be used for comparison with later disease assessments. After all screening assessments have been performed, the results reviewed, and eligibility (inclusion/exclusion) criteria confirmed, participants will enter the treatment phase.

### Treatment Phase

The Treatment Phase begins with the first administration of the study treatment and continues until the completion of the end of treatment (EOT) visit. To facilitate safety monitoring, participants may be hospitalized. Prior to and after study treatment administration, vital signs (including but not limited to pulse rate, blood pressure, oxygen saturations and temperature) measurements will be monitored at intervals noted in the SoA (Table 9). The participant will be evaluated for possible toxicities at each site visit. Bone marrow biopsy tissue will be collected during the study from participants according to the SoA (Table 9) in Parts 1 and 2 and will be shared with the local investigator to allow for central review of marrow histopathology and local disease assessment. These samples will be sent to a central laboratory designated by the sponsor.

An assessment of transfusion events will be collected on Day 1 of every cycle and at the EOT visit as per the SoA. Participants may continue to receive study treatment until any of the treatment discontinuation criteria are met. Upon discontinuation of the study treatment, the participants clinically able to return for evaluation will complete an EOT visit.

### End of Treatment Visit

The EOT visit will be completed 30±14 days after the last dose of study treatment or prior to the start of subsequent anticancer therapy, whichever comes first. The EOT visit can be completed later in case a decision is postponed due to underlying toxicity that requires longer (>30 days) follow-up. The EOT visit is required for all participants, including those who discontinue study treatment for any reason, except for lost follow-up, death, or withdrawal of consent for study participation.

### Efficacy Assessment

Efficacy assessments will include the procedures outlined in Table 9. These assessments should be performed throughout the study at each timepoint using the identical methodology used to assess disease at baseline. The investigator will perform tests that will allow evaluation of response to therapy for the corresponding disease criteria and according to the schedule outlined in Table 9. The results of these assessments should be made available preferably before the start of the next treatment target dose.

The efficacy evaluations and review of hematology and clinical chemistry results will be performed at the site level, as determined by the investigator. Response to treatment will be assessed by the investigator at the site and the results will be recorded in the eCRF. Efficacy evaluations are to be conducted until disease progression, the start of a subsequent anticancer therapy, withdrawal of consent from study participation, or the end of study, whichever comes first.

### Hematologic and Molecular Response

PB samples (approximately 10 mL of whole blood at each time point) will be collected for the assessment of hematologic and molecular burden responses from all participants at Day 1 of dosing, at 3, 6, 12, and 18 months from Day 1, and then annually or when there is suspicion of progression (+1 week) according to Table 9. Additional unscheduled laboratory monitoring should be conducted as clinically indicated to enable recognition and management of emerging signs or symptoms that may be associated with hematologic or molecular responses.

Automated cell enumeration will be performed locally. A PB smear or film will be assessed locally for morphological characterization and distribution of cells including platelets. Molecular Response assessment comprises the measurement of the CALRmut allele burden using a focused next-generation sequencing assay and will be assessed by the central laboratory.

Hemoglobin concentration in human blood can be determined before and during the treatment using methods known in the art in view of the present disclosure. See, e.g., Recommendations for reference method for haemoglobinometry in human blood (ICSH standard 1995) and specifications for international haemiglobincyanide standard (4^{th} edition). Zwart et al., 7 Clin Pathol 1996; 49:271-274, the content of which is incorporated by reference in its entirety.

### Bone Marrow Assessment

The purpose of repeated bone marrow biopsy is to permit the longitudinal assessment of megakaryocytic morphology and histological grading of fibrosis, according to European consensus grading (Thiele 2005). Bone marrow biopsies will be collected from all participants during the screening period and during treatment at 3, 6, 12, and 18 months from Day 1, and then annually or when there is suspicion of progression (+1 week) according to the SoA in Table 9. Additional unscheduled bone marrow assessments should be conducted as clinically indicated to enable recognition and management of emerging signs or symptoms that may be associated with hematologic or molecular responses.

The biopsy performed during Screening to assess morphology, immunohistochemistry (IHC), and fibrosis of core biopsies does not represent an assessment of eligibility for study enrollment but as a baseline against which later disease response assessments are judged. The provision of bone marrow biopsy specimens in the form of formalin-fixed paraffin-embedded (FFPE) blocks is requested to facilitate centralized staining and review. Unstained sections mounted on slides may be accepted with prior sponsor approval.

### Splenic Volume

A physical examination should be performed for all participants at baseline/Screening assessment, at 3, 6, 12, and 18 months from Day 1, and then annually or when there is suspicion of progression (+1 week) according to Table 9. Particular attention should be paid to assessment for enlargement of the spleen and liver. In splenectomized participants, measurements by imaging are not indicated.

Splenic volume will be assessed using computed tomography or magnetic resonance imaging (MRI) during Parts 1 and 2 of the study. With prior sponsor approval, ultrasound imaging may be used to assess splenic volume.

The method of splenic volume assessment at the site should be consistent throughout the study. Typically, a volumetric segmentation method would be used with a resulting overall spleen volume calculation. This result needs to be noted by the radiologist in the radiology report. If a volumetric method is not available at the site, the following formula should be used to estimate volume of the spleen by measuring 3 dimensions by CT or MRI: craniocaudal (L), maximum size in axial plane (D), and the maximum thickness in the axial plane (T) (Prassopoulos 1997, Eur Radiol. 7:246-248).

$$Volume ( mL ) = 30 + ( 0.58 \times L \times D \times T )$$

If ultrasound imaging is used, the volume may be estimated by the following formula comprising width (W), thickness (T), maximum length (ML) and craniocaudal length (CCL) (Yeller 2003, AJR Am J Roentgenol. 181(6):1615-20).

$$Volume ( mL ) = 0.524 \times W \times T \times ( M L + C C L ) / 2$$

In Part 2, a spleen assessment by physical examination should be performed for all participants at baseline/screening assessment and during all disease evaluation visits. This will include measurements of organomegaly below the costal margin.

### Assessment of Disease Response and Progressive Disease

Efficacy assessments for the study analyses will be performed according to modified ELN criteria for Essential Thrombocythemia (Barosi 2009, Blood. 113:4829-4833) and Myelofibrosis (Tefferi 2013, Blood. 122; 1395-1398) (see Table 11). Efficacy assessments will be performed and continue until study discontinuation criteria are met.

### Table 11: Response Assessment Criteria

TABLE 11a
Response Criteria for Participants with Essential Thrombocythemia

| Response | Parameter | | |
|---|---|---|---|
| Grade | Hematologic | Histologic | Molecular |
| Complete | Platelet count ≤400 × 10⁹/L; | Absence of | Reduction of CALRmut burden to |
| | normal spleen size on | megakaryocyte | undetectable levels |
| | imaging and white cell | hyperplasia | |
| | count ≤10 × 10⁹/L | | |
| Partial | In participants who do not | Not Applicable | 1) A reduction of ≥50% from baseline value |
| | fulfill the criteria for | | in participants with <50% mutant allele |
| | complete response, platelet | | burden at baseline OR |
| | count ≤600 × 10⁹/L or | | 2) reduction of ≥25% from baseline value in |
| | decrease >50% form | | participants with >50% mutant allele burden |
| | baseline | | at baseline |
| None | Any response that does not | Any response that | Any response that does not satisfy a partial |
| | satisfy a partial response | does not satisfy a | response |
| | | complete response | |

Abbreviations: CALRmut = calreticulin mutant.
Adapted from Barosi 2009, Blood. 113: 4829-4833

TABLE 11 b
Response Criteria for Participants with Myelofibrosis (Part 1 only)

| | Parameter | | |
|---|---|---|---|
| Response Grade | Hematologic | Histologic | Molecular |
| Complete | Hemoglobin ≥100 g/L and <ULN; | Age-adjusted | Eradication of pre-existing |
| | Neutrophil count ≥1 × 10⁹/L | normocellularity; <5% | abnormality |
| | and <ULN; | blasts; ≤Grade | |
| | Platelet count >100 × 10⁹/L | 1 MF | |
| | and <ULN | | |
| Partial | Hemoglobin ≥85 and <100 g/L | Age-adjusted | ≥50% decrease in allele burden |
| | and <ULN; | normocellularity; <5% | (if burden ≥20% at baseline) |
| | Neutrophil count ≥1 × 10⁹/L | blasts; ≤Grade | |
| | and <ULN; | 1 MF | |
| | Platelet count ≥50 and <100 | | |
| | ×10⁹/L and <ULN | | |

| | |
|---|---|
| Clinical | The achievement of anemia, spleen, or symptoms response without progressive disease or |
| Improvement | increase in severity of anemia, thrombocytopenia, or neutropenia. |
| Spleen Response | ≥35% spleen volume reduction as confirmed by imaging |
| Symptoms Response | A ≥50% reduction in the MPN-SAF TSS |
| Progressive Disease | Appearance of a new splenomegaly that is palpable at least 5 cm below the left costal |
| | margin or |
| | A ≥100% increase in palpable distance, below left costal margin, for baseline splenomegaly |
| | of 5-10 cm or |
| | A 50% increase in palpable distance, below LCM, for baseline splenomegaly of >10 cm |
| | (requires confirmation by MRI or CT showing a ≥25% increase in spleen volume from |
| | baseline) or |
| | Leukemic transformation confirmed by a bone marrow blast count of ≥20% or |
| | A peripheral blood blast content of >20% associated with an absolute blast count |
| | of >1 × 10⁹/L that lasts for at least 2 weeks |
| Stable disease | Not meeting criteria for any of the response categories listed above. |
| Relapse | No longer meeting criteria for at least clinical improvement after achieving CR, PR, or |
| | clinical improvement; or |
| | Loss of anemia response persisting for at least 1 month; or |
| | Loss of spleen response persisting for at least 1 month. |

Abbreviations: CT = computed tomography; LCM = left costal margin; MF = myelofibrosis; MRI = magnetic resonance imaging; ULN = upper limit of normal.
Adapted from Tefferi 2013, Blood. 122; 1395-1398

TABLE 11 c
Response Criteria for Participants with Myelofibrosis (Part 2 only)

| | Required criteria (for all response categories, benefit must last ≥12 weeks to |
|---|---|
| Response Categories | qualify) |
| Complete Remission | Bone marrow^{a} Age-adjusted normocellularity, <5% blasts, ≤Grade 1 MF, and |
| | Peripheral blood: Hemoglobin ≥10 g/dL and <ULN, neutrophil count ≥1 × 10⁹/L |
| | and <ULN, Platelet count ≥100 × 10⁹/L and <ULN, <2% immature myeloid cells^{b}, resolution |
| | of leukoerythroblastosis and |
| | Clinical: Resolution of disease symptoms, spleen and liver not palpable, no evidence of |
| | EMH |
| Partial Remission | Peripheral blood: Hemoglobin ≥10 g/dL and <ULN, neutrophil count ≥1 × 10⁹/L |
| | and <ULN, platelet count ≥100 × 10⁹/L |
| | and <ULN, <2% immature myeloid cells^{b}, and |
| | Clinical: Resolution of disease symptoms, spleen and liver not palpable, no evidence of |
| | EMH, or |
| | Bone marrow: Age-adjusted normocellularity, <5% blasts, ≤Grade 1 MF, and |
| | Peripheral blood: Hemoglobin ≥8.5 but <10 mg/L and <ULN, neutrophil count ≥1 × |
| | 10⁹/L and <ULN, platelet count ≥50, but <100 × 10⁹/L and <ULN, <2% immature |
| | myeloid cells^{b}, and |
| | Clinical: Resolution of disease symptoms, spleen and liver not palpable, no evidence of |
| | EMH |
| Clinical Improvement | The achievement of anemia, spleen, or symptoms response without progressive disease |
| | or increase in severity of anemia, thrombocytopenia, or neutropeniac. |
| Anemia Response | Transfusion-independent patients: ≥2 g/dL increase in hemoglobin level^{d} Transfusion- |
| | dependent patients: becoming transfusion-independent. |
| Spleen Response^{e} | A baseline splenomegaly that is palpable at 5-10 cm, below the LCM, becomes not |
| | palpable^{f} or |
| | A baseline splenomegaly that is palpable at >10 cm, below the LCM, decreases by ≥50%^{f} |
| | A baseline splenomegaly that is palpable at <5 cm, below the LCM, is not eligible for |
| | spleen response |
| | A spleen response requires confirmation by ultrasound or other imaging modality |
| | showing ≥35% spleen volume reduction |
| Symptoms Response | A ≥50% reduction in the MPN-SAF TSS |
| Progressive Disease | Appearance of a new splenomegaly that is palpable at least 5 cm below the left costal |
| | margin or |
| | A ≥100% increase in palpable distance, below left costal margin, for baseline |
| | splenomegaly of 5-10 cm or |
| | A 50% increase in palpable distance, below LCM, for baseline splenomegaly of >10 |
| | cm or |
| | Leukemic transformation confirmed by a bone marrow blast count of ≥20% or |
| | A peripheral blood blast content of >20% associated with an absolute blast count |
| | of >1 × 10⁹/L that lasts for at least 2 weeks |
| Stable disease | Belonging to none of the above listed response categories |
| Relapse | No longer meeting criteria for at least clinical improvement after achieving CR, PR, or |
| | clinical improvement; or |
| | Loss of anemia response persisting for at least 1 month; or |
| | Loss of spleen response persisting for at least 1 month. |

Recommendations for assessing treatment-induced cytogenetic and molecular changes

| | |
|---|---|
| Cytogenetic Remission | At least 10 metaphases must be analyzed for cytogenetic response evaluation and requires |
| | confirmation by repeat testing within 6 months window |
| | CR: Eradication of a pre-existing abnormality |
| | PR: ≥50% reduction in abnormal metaphases |
| | (partial response applies only to patients with at least 10 abnormal metaphases at |
| | baseline) |
| Molecular Remission | Molecular response evaluation must be analyzed in peripheral blood granulocytes and |
| | requires confirmation by repeat testing within 6 months window |
| | CR: Eradication of a pre-existing abnormality |
| | PR: ≥50% decrease in allele burden |
| | (partial response applies only to patients with at least 20% mutant allele burden at |
| | baseline) |
| Cytogenetic/Molecular | Re-emergence of a pre-existing cytogenetic or molecular abnormality that is confirmed |
| Relapse | by repeat testing |

^{a}Cytogenetic and molecular responses are not required for CR assignment.
^{b}Immature myeloid cells constitute blasts + promyelocytes + myelocytes + metamyelocytes + nucleated red blood cells. In splenectomized patients, <5% immature myeloid cells is allowed.
^{c}Increase in severity of anemia constitutes the occurrence of new transfusion dependency or a ≥2 g/dL decrease in hemoglobin level from pretreatment baseline that lasts for ≥12 weeks. Increase in severity of thrombocytopenia or neutropenia is defined as a 2-grade decline, from pretreatment baseline, in platelet count or absolute neutrophil count, according to the CTCAE version 5.0. In addition, assignment to CI requires a minimum platelet count of ≥25,000 × 10(9)/L and absolute neutrophil count of ≥0.5 × 10(9)/L.
^{d}Applicable only to patients with baseline hemoglobin of <10 g/dL. In patients not meeting the strict criteria for transfusion dependency at the time of study enrollment, but have received transfusions within the previous month, the pretransfusion hemoglobin level should be used as the baseline.
^{e}In splenectomized patients, palpable hepatomegaly is substituted with the same measurement strategy.
^{f}Spleen or liver responses (to be applicable, baseline splenomegaly or hepatomegaly must be palpable) must be confirmed by imaging studies where a ≥35% reduction in spleen volume, as assessed by ultrasound (or other imaging modality), is required. Furthermore, a ≥35% volume reduction in the spleen or liver, by ultrasound or other imaging modality, constitutes a response regardless of what is reported with physical examination.
^{g}Progressive disease assignment for splenomegaly requires confirmation by ultrasound or other imaging modality showing a ≥25% increase in spleen volume from baseline.
Adapted from Tefferi 2013, Blood. 122; 1395-1398

### Safety Assessments

Safety will be monitored by the SET. Safety assessments will be measured by adverse events (AEs), clinical laboratory test results, ECGs, vital sign measurements, physical examination findings (including basic neurologic exam), and assessment of ECOG performance status score at the timepoints in Table 9. Safety monitoring (clinical evaluations and laboratory assessments) may be performed more frequently, if clinically indicated, and AEs should be evaluated by the investigator according to the standard practice.

### Adverse Events

AEs will be reported and followed by the investigator, and AEs will be graded according to the NCI-CTCAE, Version 5.0, except for CRS and ICANS which will be graded by ASCTC. Any clinically relevant changes occurring during the study must be recorded in the AE section of the eCRF.

The study will include the following evaluations of safety and tolerability according to the time points provided in the SoA (Table 9).

Adverse events of special interest are systemic administration-related reaction (sARR), neurotoxicity, and cytokine release syndrome (CRS) of Grade ³3 will be followed as AESIs.

### F. Analytical Procedures

### Pharmacokinetics

Serum samples will be analyzed to determine concentrations of COMPOUND 1 using a validated, specific, and sensitive immunoassay method by or under the supervision of the sponsor.

PK parameters will be estimated for individuals, and descriptive statistics will be calculated for each dose level. PK parameters include but are not limited to Cmax, Ctrough, Tmax, T1/2, AUC0-t, AUC0-τ, apparent total body clearance (CL/F), and apparent volume of distribution (V/F); parameters will be calculated if sufficient data are available for estimation. In addition, exploratory population PK-based approach may also be applied for PK analysis. The results of the population PK analysis will be reported separately.

### Immunogenicity

The detection and characterization of anti-COMPOUND 1 antibodies will be performed using a validated assay method by or under the supervision of the sponsor. All serum samples collected for detection of anti-COMPOUND 1 antibodies will also be evaluated for serum COMPOUND 1 concentration to enable interpretation of the antibody data. Immune response analysis may be conducted on PK samples collected at other time points. Additionally, serum samples should also be collected at the final visit from participants who are discontinued from treatment or withdraw from the study. These samples will be tested by the sponsor or sponsor's designee.

Serum samples will be screened for antibodies binding to COMPOUND 1 and the titer of confirmed positive samples will be reported. The ADA positive samples may be tested further for neutralizing antibodies to COMPOUND 1. Immune response analysis may be conducted on PK samples collected at other timepoints, if deemed necessary. Other analyses may be performed to characterize immunogenicity.

### Receptor Occupancy (RO)

Whole blood samples will be collected and analyzed for RO assessment to quantify the binding of COMPOUND 1 to CALRmut on the cell surface via flow cytometry. RO samples will be collected in Part 2 if the RO sample results from Part 1 are comprehensive and meaningful for study treatment response assessments.

### Soluble CALRmut

Soluble CALRmut levels in serum will be measured to explore its relationship with PK, safety, and efficacy of COMPOUND 1. Serum samples will be analyzed to determine soluble CALRmut concentrations using Meso Scale Discovery (MSD) ECLIA method. Serum samples for soluble CALRmut assessment will be collected in Part 1 and Part 2.

### Biomarkers

Biomarker samples will be collected to determine PD markers indicative of the MOA for COMPOUND 1 and to evaluate mechanisms of response/resistance.

Samples collected for biomarker evaluations will include a bone marrow biopsy and PB samples (Table 12). These samples may be analyzed by technologies including but not limited to next-generation sequencing analysis and proteomics. Flow cytometry-based evaluations or Cytometry by Time-of-Flight (CYTOF) of immune cells subsets from PB will also be performed to determine exploratory biomarkers and to evaluate the hypothesis that the treatment may be modulated by the T-cell phenotype. Serum fraction isolated from blood will be used for analysis of cytokines (including but not limited to IFN-γ, IL-2, IL-6, and IL-10), chemokines, or other secreted proteins. Samples may be used to help address emerging issues and enable the development of safer, more effective therapies. Biological samples collected on this study may be used to develop companion diagnostics. The analyses planned for the samples collected during the study are provided in the table below.

TABLE 12
Pharmacodynamic Biomarkers

| Material | Analysis |
|---|---|
| Bone Marrow | H&E and ancillary studies (IHC and special stains; |
| Biopsy (FFPE | age-adjusted cellularity, megakaryocyte hyperplasia, |
| block) | fibrosis, CALRmut, CD3, CD34, CD61) and |
| | others as required to determine hematopoietic |
| | recovery based on emerging data) |
| Peripheral | Serum Biochemical parameters: including |
| Blood - cellular | but not exclusive to Ferritin, LDH, C- |
| fractions and serum | Reactive protein. |
| | Mutation profiling for |
| | CALRmut VAF and co-mutations |
| | Proteomics (cytokines, chemokines & |
| | soluble receptors) |
| | Lymphocyte subsets by |
| | immunophenotyping and/or CYTOF |
| | Circulating CD34+ cells |

Abbreviations: CALRmut = calreticulin mutant; CD = cluster of differentiation; CYTOF = Cytometry by Time-of-Flight; H&E = Hematoxylin and eosin; IHC = immunohistochemistry; FFPE = formalin-fixed, paraffin-embedded; LDH = lactate dehydrogenase; VAF = variant allele frequency.

### Immunophenotyping

Bispecific antibodies function by engaging T cells, leading to T-cell activation, margination, expansion, and tumor killing activity. Excessive stimulation of the TCR and robust co-inhibitory molecule signaling or restricted presence of concomitant co-stimulation through CD28 can lead to T-cell exhaustion and lack of clinical responses to T-cell engagers. Consequently, markers of T-cell activation and exhaustion will be evaluated, and the proportion of T-cell subsets will be enumerated to help identify a dose and course that might optimize therapeutic potential.

### Pro-Inflammatory Cytokines

Activated T cells produce certain pro-inflammatory cytokines to induce cytotoxicity and immune responses. Therefore, pro-inflammatory cytokines will be measured to corroborate MOA. In addition, as chronic inflammation is a hallmark of MPN and increases in cytokine release can lead to CRS events, the levels of pro-inflammatory cytokines will be monitored for safety and to implement mitigation strategies when needed.

### Mutational Profiling

The variant allele frequency of mutant CALR will be monitored in peripheral blood to correlate mutational burden with clinical responses. In addition, numerous disease-specific co-mutations or cytogenetic abnormalities are common during disease progression. These mutations can drive severity of disease and promote resistance to numerous therapies. Therefore, it will be important to explore whether specific genetic alterations correlate with antitumor activity of the study treatment.

### Immunohistochemistry

To evaluate bone marrow responses, fibrosis, megakaryocyte morphology, and presence of mutant CALR+, CD34+, and T cells will be reported centrally in bone marrow biopsies by H&E, Special stains and IHC.

### Predictive Biomarkers

To identify predictors of response, clinical outcomes will be correlated with mutant CALR burden, presence of co-mutations, cytokine expression, and the level of immune cell subsets within tumor samples from whole blood at baseline. Other biomarkers may also be explored if new data emerges. These results may predict markers of clinical response or resistance to guide future studies.

### G. Sample Size Determination

Part 1: It is anticipated that 40-60 participants will be enrolled in Part 1; the total number of participants enrolled in Part 1 will depend on the number of cohorts explored to identify the putative RP2D(s) and the number of participants who will be enrolled in each cohort.

Part 2: Up to approximately 20 participants will be enrolled for each disease category cohort at each candidate RP2D and selected schedule in Part 2.

The total number of participants enrolled in the clinical trial, including both in Part 1 and Part 2, is anticipated to be up to approximately 100 participants.

Regarding the sample size determination for Part 2 and the evaluation of clinical activity or an AE rate with 20 participants in a cohort, 95% exact confidence intervals for several of potential observed numbers of responses or AEs are provided in Table 13.

TABLE 13
Observed Response or Adverse Event Rates and
95% Confidence Intervals (Sample Size of 20)

| Number of Responses or | Observed Response Rate or | 95% Confidence |
|---|---|---|
| Adverse Events | Adverse Event Rate | Interval |
| 2 | 10% | (1%, 32%) |
| 3 | 15% | (3%, 38%) |
| 4 | 20% | (6%, 44%) |
| 5 | 25% | (9%, 49%) |
| 6 | 30% | (12%, 54%) |

### H. Statistical Analysis

### Primary Endpoints

The primary endpoint is the frequency of DLTs and the frequency and severity of AEs.

The PK analysis set will be used. All serum COMPOUND 1 concentrations below the lowest quantifiable concentration or missing data will be labeled as such in the concentration data presentations or Statistical Analysis System (SAS) dataset. Serum COMPOUND 1 concentrations below the lower quantifiable concentration will be treated as zero in the summary statistics.

Descriptive statistics will be used to summarize serum COMPOUND 1 concentrations at each sampling timepoint and PK parameters of COMPOUND 1: AUC0-τ, Ctrough, and Cmax. Other PK parameters, including but not limited to Tmax, T1/2, AUC0-t, CL/F, and V/F, when available, will also be summarized. Mean (standard deviation) serum COMPOUND 1 concentration-time profiles will be plotted after the study treatment administration, and individual serum COMPOUND 1 concentration-time profiles may also be plotted.

Immunogenicity analyses will be descriptive in nature and will include the number and percentage of participants who developed anti-COMPOUND 1 antibodies. A listing of participants who are positive for antibodies to COMPOUND 1 will be provided. The maximum titers of antibodies to COMPOUND 1 will be summarized for participants who are positive for antibodies to COMPOUND 1. The impact of anti-COMPOUND 1 antibodies on PK, safety, and efficacy may also be evaluated.

Analyses are planned to identify biomarkers that are indicative of the MOA of the study treatment, or predictive of efficacy and resistance. The associations of biomarkers with clinical response or time-to-event endpoints will be assessed using the appropriate statistical methods (analysis of variance [ANOVA], categorical, or survival model), depending on the endpoint. Correlation of baseline expression levels or changes in expression levels with response or time-to-event endpoints will identify responsive or resistant subgroups.

### Secondary Endpoints

In Part 1 (dose escalation), the primary markers of response will be the hematologic, molecular, histologic, and splenic response. Part 2 (cohort expansion) will also incorporate a measure of symptom burden according to an appropriate Patient Reported Outcome (PRO) questionnaire.

Response rate will be tabulated, together with its 2-sided 95% exact confidence interval. In addition, the number and percentage of participants in each response category will be tabulated. Time to Response (TTR) and DOR estimates will be provided using Kaplan-Meier method. These will be applied to the components of response according to ELN 2013 as well as Overall Response Rate.

The efficacy endpoints are:
Overall Response Rate (ORR): ORR is defined as the proportion of participants who have a PR and CR according to the disease criteria. Response to treatment with the study treatment will be evaluated by the investigator.
Complete Response Rate (CRR): CR rate is defined as the proportion of participants who achieve a best response of CR according to disease.
Time to Response (TTR): TTR is defined for participants who achieved PR or CR as the time from the first dose of study treatment to the first response of PR or CR.
Duration of Response (DOR): DOR is defined for participants who achieved PR or CR as the time between the date of initial documentation of PR or CR to the date of first documented evidence of disease progression or death, whichever comes first. For participants who have not progressed or died, data will be censored at the last disease evaluation.

### Results

The study is ongoing. No unexpected safety or tolerability issues were identified from the interim safety and tolerability results. All patients experienced low grade injection site reactions (ISR). All grade 1 except one case of grade 2 ISR. Some patients had reoccurrences of ISR with subsequent doses, with improvement over time. No increase in peak ISR grade with dose escalation from 0.6 mg to 4.8 mg (8-fold). Low grade CRS was observed in some patients. Step up strategy was implemented for all new cohorts to mitigate ISR and CRS.

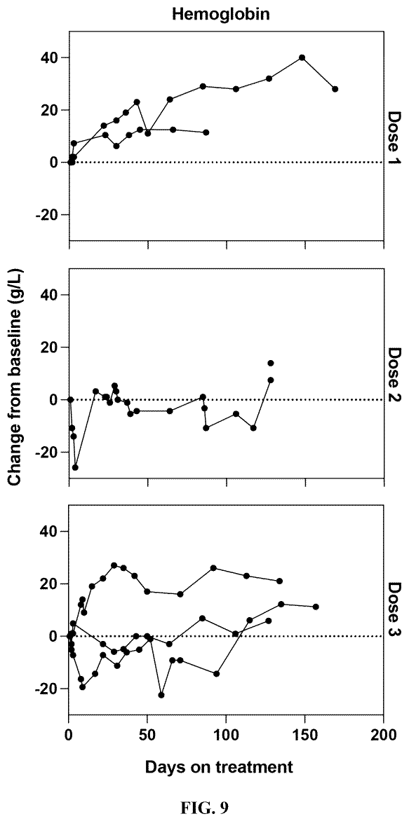

Early efficacy observations guided dose escalation. Accumulating evidence showed benefit from 2.4 mg dosing upwards. Cohort results showed the recovery of hemoglobin following treatment with COMPOUND 1. See, e.g., Table 14, and FIG. 9.

TABLE 14
Hemoglobin changes from the baseline of subjects
treated at various doses of COMPOUND 1.

| | | Day of | Hemoglobin | Change from baseline |
|---|---|---|---|---|
| Dose | Participant | treatment | (g/L) | (g/L) |
| Dose 1 | 4 | 1 | 80 | 0 |
| | | 2 | 80 | 0 |
| | | 3 | 82 | 2 |
| | | 22 | 94 | 14 |
| | | 30 | 96 | 16 |
| | | 36 | 99 | 19 |
| | | 43 | 103 | 23 |
| | | 50 | 91 | 11 |
| | | 64 | 104 | 24 |
| | | 85 | 109 | 29 |
| | | 106 | 108 | 28 |
| | | 127 | 112 | 32 |
| | | 148 | 120 | 40 |
| | | 169 | 108 | 28 |
| | 10 | 1 | 96 | 0 |
| | | 2 | 98 | 2 |
| | | 3 | 103 | 7 |
| | | 23 | 106 | 10 |
| | | 30 | 102 | 6 |
| | | 38 | 106 | 10 |
| | | 45 | 108 | 13 |
| | | 66 | 108 | 13 |
| | | 87 | 107 | 11 |
| Dose 2 | 2 | 1 | 93 | 0 |
| | | 2 | 83 | −11 |
| | | 3 | 80 | −14 |
| | | 4 | 69 | −26 |
| | | 17 | 96 | 3 |
| | | 23 | 94 | 1 |
| | | 24 | 94 | 1 |
| | | 26 | 92 | −1 |
| | | 29 | 98 | 5 |
| | | 30 | 96 | 3 |
| | | 31 | 93 | 0 |
| | | 37 | 92 | −1 |
| | | 39 | 88 | −5 |
| | | 43 | 89 | −4 |
| | | 64 | 89 | −4 |
| | | 85 | 94 | 1 |
| | | 86 | 90 | −3 |
| | | 87 | 83 | −11 |
| | | 106 | 88 | −5 |
| | | 117 | 83 | −11 |
| | | 128 | 100 | 8 |
| | | 128 | 106 | 14 |
| Dose 3 | 5 | 1 | 98 | 0 |
| | | 2 | 93 | −5 |
| | | 3 | 91 | −7 |
| | | 8 | 82 | −16 |
| | | 9 | 79 | −19 |
| | | 17 | 84 | −14 |
| | | 22 | 91 | −7 |
| | | 31 | 87 | −11 |
| | | 37 | 92 | −6 |
| | | 45 | 93 | −5 |
| | | 52 | 97 | −1 |
| | | 59 | 76 | −22 |
| | | 66 | 89 | −9 |
| | | 71 | 89 | −9 |
| | | 94 | 84 | −14 |
| | | 115 | 104 | 6 |
| | | 135 | 110 | 12 |
| | | 157 | 109 | 11 |
| | 6 | 1 | 102 | 0 |
| | | 2 | 99 | −3 |
| | | 3 | 107 | 5 |
| | | 22 | 99 | −3 |
| | | 29 | 96 | −6 |
| | | 35 | 97 | −5 |
| | | 43 | 102 | 0 |
| | | 50 | 102 | 0 |
| | | 64 | 99 | −3 |
| | | 85 | 109 | 7 |
| | | 106 | 103 | 1 |
| | | 127 | 108 | 6 |
| | | 1 | 80 | 0 |
| | | 2 | 81 | 1 |
| | | 3 | 81 | 1 |
| | | 8 | 92 | 12 |
| | | 9 | 94 | 14 |
| | | 10 | 89 | 9 |
| | | 15 | 99 | 19 |
| | 8 | 22 | 102 | 22 |
| | | 29 | 107 | 27 |
| | | 35 | 106 | 26 |
| | | 42 | 103 | 23 |
| | | 50 | 97 | 17 |
| | | 71 | 96 | 16 |
| | | 92 | 106 | 26 |
| | | 113 | 103 | 23 |
| | | 134 | 101 | 21 |

Dose 1: 1.2 mg
Dose 2: 1.8 mg
Dose 3: 2.4 mg (with or without Step-Up)
Negative values indicate a reduction in Hemoglobin levels from baseline
Baseline is the Hemoglobin value at pre-dose on day 1 of treatment.

SEQUENCE LISTING

| SEQ | |
|---|---|
| ID | |
| NO: | Sequence |
| 1 | KMSPARPRTSCREACLQGWTEA |
| 2 | RRKMSPARPRTSCREACLQGWTEA |
| 3 | EPAVYFKEQFLDGDGWTSRWIESKHKSDFGKFVLSSGKFYGDEEKDKGLQTSQDARFYALSAS |
| | FEPFSNKGQTLVVQFTVKHEQNIDCGGGYVKLFPNSLDQTDMHGDSEYNIMFGPDICGPGTKK |
| | VHVIFNYKGKNVLINKDIRCKDDEFTHLYTLIVRPDNTYEVKIDNSQVESGSLEDDWDFLPPKKI |
| | KDPDASKPEDWDERAKIDDPTDSKPEDWDKPEHIPDPDAKKPEDWDEEMDGEWEPPVIQNPEY |
| | KGEWKPRQIDNPDYKGTWIHPEIDNPEYSPDPSIYAYDNFGVLGLDLWQVKSGTIFDNFLITNDE |
| | AYAEEFGNETWGVTKAAEKQMKDKQDEEQRLKEEEEDKKRKEEEEAEDNCRRMMRTKMRM |
| | RRMRRTRRKMRRKMSPARPRTSCREACLQGWTEADYKDDDDK |
| 4 | EPAVYFKEQFLDGDGWTSRWIESKHKSDFGKFVLSSGKFYGDEEKDKGLQTSQDARFYALSAS |
| | FEPFSNKGQTLVVQFTVKHEQNIDCGGGYVKLFPNSLDQTDMHGDSEYNIMFGPDICGPGTKK |
| | VHVIFNYKGKNVLINKDIRCKDDEFTHLYTLIVRPDNTYEVKIDNSQVESGSLEDDWDFLPPKKI |
| | KDPDASKPEDWDERAKIDDPTDSKPEDWDKPEHIPDPDAKKPEDWDEEMDGEWEPPVIQNPEY |
| | KGEWKPRQIDNPDYKGTWIHPEIDNPEYSPDPSIYAYDNFGVLGLDLWQVKSGTIFDNFLITNDE |
| | AYAEEFGNETWGVTKAAEKQMKDKQDEEQRLKEEEEDKKRKEEEEAEDKEDDEDKDEDEED |
| | EEDKEEDEEEDVPGQAKDELDYKDDDDK |
| 5 | MRGSHHHHHHGMASMTGGQQMGRDLYDDDDKDRWGSELEAEPAVYFKEQFLDGDGWTSR |
| | WIESKHKSDFGKFVLSSGKFYGDEEKDKGLQTSQDARFYALSASFEPFSNKGQTLVVQFTVKHE |
| | QNIDCGGGYVKLFPNSLDQTDMHGDSEYNIMFGPDICGPGTKKVHVIFNYKGKNVLINKDIRCK |
| | DDEFTHLYTLIVRPDNTYEVKIDNSQVESGSLEDDWDFLPPKKIKDPDASKPEDWDERAKIDDP |
| | TDSKPEDWDKPEHIPDPDAKKPEDWDEEMDGEWEPPVIQNPEYKGEWKPRQIDNPDYKGTWI |
| | HPEIDNPEYSPDPSIYAYDNFGVLGLDLWQVKSGTIFDNFLITNDEAYAEEFGNETWGVTKAAE |
| | KQMKDKQDEEQRTRRMMRTKMRMRRMRRTRRKMRRKMSPARPRTSCREACLQGWTEA |
| 6 | MGSHHHHHHGSSGEPAVYFKEQFLDGDGWTSRWIESKHKSDFGKFVLSSGKFYGDEEKDKGL |
| | QTSQDARFYALSASFEPFSNKGQTLVVQFTVKHEQNIDCGGGYVKLFPNSLDQTDMHGDSEYN |
| | IMFGPDICGPGTKKVHVIFNYKGKNVLINKDIRSKDDEFTHLYTLIVRPDNTYEVKIDNSQVESG |
| | SLEDDWDFLPPKKIKDPDASKPEDWDERAKIDDPTDSKPEDWDKPEHIPDPDAKKPEDWDEEM |
| | DGEWEPPVIQNPEYKGEWKPRQIDNPDYKGTWIHPEIDNPEYSPDPSIYAYDNFGVLGLDLWQV |
| | KSGTIFDNFLITNDEAYAEEFGNETWGVTKAAEKQMKDKQDEEQRTRRMMRTKMRMRRMRR |
| | TRRKMRRKMSPARPRTSCREACLQGWTEA |
| 7 | QSSQSVYNNNWLS |
| 8 | DASKLES |
| 9 | AGGFTGNVYT |
| 10 | NNAIS |
| 11 | GIDLSNNAIS |
| 12 | VIGNTGDTY |
| 13 | GPPSYSSSVKNI |
| 14 | EVQLLESGGGLVQPGGSLRLSCAVSGIDLSNNAISWVRQAPGKGLEYVGVIGNTGDTYYADSA |
| | KGRFTISRDSSKTTLYLQMNSLRAEDTAVYFCVRGPPSYSSSVKNIWGQGTLVTVSS |
| 15 | EVQLLESGGGLVQPGGSLRLSCAVSGIDLSNNAISWVRQAPGKGLEYVGVIGNTGDTYYADSA |
| | KGRFTISRDSSKTTLYLQMNSLRAEDTAVYFCVRGPPSYSSSVKNIWGQGTLVTVSSASTKGPS |
| | VFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLSCAVKGFYPSDIAVE |
| | WESNGQPENNYKTTPPVLDSDGSFFLVSKLTVDKSRWQQGNVFSCSVMHEALHNRFTQKSLSL |
| | SPGK |
| 16 | DIQMTQSPSSVSASVGDRVTITCQSSQSVYNNNWLSWLQQKPGKAPKRLIYDASKLESGVPSRF |
| | SGSGSGTDFTLTISSVQPEDAATYYCAGGFTGNVYTFGGGTKVEIK |
| 17 | DIQMTQSPSSVSASVGDRVTITCQSSQSVYNNNWLSWLQQKPGKAPKRLIYDASKLESGVPSRF |
| | SGSGSGTDFTLTISSVQPEDAATYYCAGGFTGNVYTFGGGTKVEIKRTVAAPSVFIFPPSDEQLK |
| | SGTASVVCLLNNFYPREAKVQWKVDNALQSGNSQESVTEQDSKDSTYSLSSTLTLSKADYEKH |
| | KVYACEVTHQGLSSPVTKSFNRGEC |
| 18 | ATGGCCAGGAAGTCCGCTCTGCTCGCTCTGGCACTTCTGCTTCTGGGATTTGGACCTGCTTG |
| | GGCTGAGGTTCAGCTGCTGGAATCTGGCGGAGGATTGGTTCAGCCTGGCGGCTCTCTGAGA |
| | CTGTCTTGTGCTGTGTCTGGCATCGACCTGTCCAACAACGCCATCTCCTGGGTCCGACAGGC |
| | TCCTGGCAAAGGCCTGGAATATGTGGGCGTGATCGGCAACACCGGCGACACCTACTACGCC |
| | GATTCTGCCAAGGGCAGATTCACCATCTCTCGGGACTCCTCCAAGACCACACTGTACCTGC |
| | AGATGAACTCCCTGAGAGCCGAGGATACCGCCGTGTACTTTTGTGTGCGGGGACCTCCTAG |
| | CTACTCCTCCAGCGTGAAGAACATCTGGGGCCAGGGCACACTGGTCACCGTTTCTTCTGCCT |
| | CCACCAAGGGCCCATCGGTCTTCCCCCTGGCACCCTCCTCCAAGAGCACCTCTGGGGGCAC |
| | AGCGGCCCTGGGCTGCCTGGTCAAGGACTACTTCCCCGAACCGGTGACGGTGTCGTGGAAC |
| | TCAGGCGCCCTGACCAGCGGCGTGCACACCTTCCCGGCTGTCCTACAGTCCTCAGGACTCTA |
| | CTCCCTCAGCAGCGTGGTGACCGTGCCCTCCAGCAGCTTGGGCACCCAGACCTACATCTGC |
| | AACGTGAATCACAAGCCCAGCAACACCAAGGTGGACAAGAAAGTTGAGCCCAAATCTTGT |
| | GACAAAACTCACACATGCCCACCGTGCCCAGCACCTGAAGCCGCCGGGGGACCGTCAGTCT |
| | TCCTCTTCCCCCCAAAACCCAAGGACACCCTCATGATCTCCCGGACCCCTGAGGTCACATGC |
| | GTGGTGGTGAGCGTGAGCCACGAAGACCCTGAGGTCAAGTTCAACTGGTACGTGGACGGC |
| | GTGGAGGTGCATAATGCCAAGACAAAGCCGCGGGAGGAGCAGTACAACAGCACGTACCGT |
| | GTGGTCAGCGTCCTCACCGTCCTGCACCAGGACTGGCTGAATGGCAAGGAGTACAAGTGCA |
| | AGGTGTCGAACAAAGCCCTCCCAGCCCCCATCGAGAAAACCATCTCCAAAGCCAAAGGGC |
| | AGCCCCGAGAACCACAGGTGTACACCCTGCCCCCATCCCGGGAGGAGATGACCAAGAACC |
| | AGGTCAGCCTGTCCTGCGCCGTCAAAGGCTTCTATCCCAGCGACATCGCCGTGGAGTGGGA |
| | GAGCAATGGGCAGCCGGAGAACAACTACAAGACCACGCCTCCCGTGCTGGACTCCGACGG |
| | CTCCTTCTTCCTCGTGAGCAAGCTCACCGTGGACAAGAGCAGATGGCAGCAGGGGAACGTC |
| | TTCTCATGCTCCGTGATGCATGAGGCTCTGCACAACCGGTTCACGCAGAAGTCTCTCTCCCT |
| | GTCTCCGGGAAAA |
| 19 | ATGGCCAGGAAGTCCGCTCTGCTCGCTCTGGCACTTCTGCTTCTGGGATTTGGACCTGCTTG |
| | GGCTGACATCCAGATGACCCAGTCTCCATCCTCCGTGTCTGCCTCTGTGGGCGACAGAGTG |
| | ACCATCACCTGTCAGTCCAGCCAATCCGTGTACAACAACAACTGGCTGTCCTGGCTGCAGC |
| | AGAAGCCTGGCAAGGCTCCCAAGAGACTGATCTACGACGCCTCCAAGCTGGAATCCGGCGT |
| | GCCCTCCAGATTTTCCGGCTCTGGCTCTGGCACCGACTTTACCCTGACCATCTCCTCCGTGC |
| | AGCCTGAGGATGCCGCCACCTATTATTGTGCTGGAGGCTTCACCGGCAACGTGTACACATTT |
| | GGCGGAGGCACCAAGGTGGAAATCAAGCGTACTGTGGCTGCACCATCTGTCTTCATCTTCC |
| | CGCCATCTGATGAGCAGTTGAAATCTGGAACTGCCTCTGTTGTGTGCCTGCTGAATAACTTC |
| | TATCCCAGAGAGGCCAAAGTACAGTGGAAGGTGGATAACGCCCTCCAATCGGGTAACTCCC |
| | AGGAGAGTGTCACAGAGCAGGACAGCAAGGACAGCACCTACAGCCTCAGCAGCACCCTGA |
| | CGCTGAGCAAAGCAGACTACGAGAAACACAAAGTCTACGCCTGCGAAGTCACCCATCAGG |
| | GCCTGAGCTCGCCCGTCACAAAGAGCTTCAACAGGGGAGAGTGT |
| 20 | GFTFSRYNMN |
| 21 | SISTSSNYIY |
| 22 | GWGPFDY |
| 23 | EVQLVESGGGLVKPGGSLRLSCAASGFTFSRYNMNWVRQAPGKGLEWVSSISTSSNYIYYADS |
| | VKGRFTFSRDNAKNSLDLQMSGLRAEDTAIYYCTRGWGPFDYWGCGTLVTVSS |
| 24 | RARQSIGTAIH |
| 25 | YASESIS |
| 26 | QQSGSWPYT |
| 27 | DIQMTQSPSSLSASVGDRVTITCRARQSIGTAIHWYQQKPGCAPKLLIKYASESISGVPSRFSGSG |
| | SGTDFTLTISSLQPEDFATYYCQQSGSWPYTFGQGTKLEIK |
| 28 | DIQMTQSPSSLSASVGDRVTITCRARQSIGTAIHWYQQKPGCAPKLLIKYASESISGVPSRFSGSG |
| | SGTDFTLTISSLQPEDFATYYCQQSGSWPYTFGQGTKLEIKGGGSGGSGGCPPCGGSGGEVQLV |
| | ESGGGLVKPGGSLRLSCAASGFTFSRYNMNWVRQAPGKGLEWVSSISTSSNYIYYADSVKGRF |
| | TFSRDNAKNSLDLQMSGLRAEDTAIYYCTRGWGPFDYWGCGTLVTVSSEPKSSDKTHTCPPCP |
| | APEAAGGPSVFLFPPKPKDTLMISRTPEVTCVVVSVSHEDPEVKFNWYVDGVEVHNAKTKPRE |
| | EQYNSTYRVVSVLTVLHQDWLNGKEYKCKVSNKALPAPIEKTISKAKGQPREPQVYTLPPSREE |
| | MTKNQVSLWCLVKGFYPSDIAVEWESNGQPENNYKTTPPVLDSDGSFFLYSKLTVDKSRWQQ |
| | GNVFSCSVMHEALHNHYTQKSLSLSPGK |
| 29 | ATGGCCAGGAAGTCCGCTCTGCTCGCTCTGGCACTTCTGCTTCTGGGATTTGGACCTGCTTG |
| | GGCTGACATCCAGATGACCCAGTCTCCATCCTCTCTGTCCGCCTCTGTGGGCGACAGAGTGA |
| | CCATTACCTGCCGGGCCAGACAGTCTATCGGCACCGCTATCCACTGGTATCAGCAGAAGCC |
| | TGGCTGTGCCCCTAAGCTGCTGATTAAGTACGCCTCCGAGTCCATCTCCGGCGTGCCCTCCA |
| | GATTTTCTGGCTCTGGATCTGGCACCGACTTTACCCTGACAATCTCCAGCCTGCAGCCTGAG |
| | GACTTCGCCACCTACTACTGTCAGCAGTCCGGCTCTTGGCCTTACACCTTTGGTCAGGGCAC |
| | CAAGCTGGAAATCAAGGGCGGAGGTTCTGGTGGATCTGGCGGATGTCCTCCTTGTGGTGGA |
| | AGCGGCGGAGAGGTGCAGCTGGTTGAATCTGGCGGAGGACTGGTTAAGCCTGGCGGCTCTC |
| | TGAGACTGTCTTGTGCTGCTTCTGGCTTCACCTTCAGCCGGTACAACATGAACTGGGTCCGA |
| | CAGGCTCCTGGCAAAGGCCTGGAATGGGTGTCCTCCATCTCCACCTCCAGCAACTACATCT |
| | ACTACGCCGACTCCGTGAAGGGCAGATTCACCTTCTCCAGAGACAACGCCAAGAACTCCCT |
| | GGACCTGCAGATGTCTGGCCTGAGAGCTGAGGACACCGCTATCTACTACTGCACCAGAGGC |
| | TGGGGACCCTTCGATTATTGGGGCTGTGGAACCCTGGTCACCGTGTCATCTGAGCCCAAATC |
| | TAGCGACAAAACTCACACATGCCCACCGTGCCCAGCACCTGAAGCCGCCGGGGGACCGTCA |
| | GTCTTCCTCTTCCCCCCAAAACCCAAGGACACCCTCATGATCTCCCGGACCCCTGAGGTCAC |
| | ATGCGTGGTGGTGAGCGTGAGCCACGAAGACCCTGAGGTCAAGTTCAACTGGTACGTGGAC |
| | GGCGTGGAGGTGCATAATGCCAAGACAAAGCCGCGGGAGGAGCAGTACAACAGCACGTAC |
| | CGTGTGGTCAGCGTCCTCACCGTCCTGCACCAGGACTGGCTGAATGGCAAGGAGTACAAGT |
| | GCAAGGTGTCGAACAAAGCCCTCCCAGCCCCCATCGAGAAAACCATCTCCAAAGCCAAAG |
| | GGCAGCCCCGAGAACCACAGGTGTACACCCTGCCCCCATCCCGGGAGGAGATGACCAAGA |
| | ACCAGGTCAGCCTGTGGTGCCTGGTCAAAGGCTTCTATCCCAGCGACATCGCCGTGGAGTG |
| | GGAGAGCAATGGGCAGCCGGAGAACAACTACAAGACCACGCCTCCCGTGCTGGACTCCGA |
| | CGGCTCCTTCTTCCTCTACAGCAAGCTCACCGTGGACAAGAGCAGATGGCAGCAGGGGAAC |
| | GTCTTCTCATGCTCCGTGATGCATGAGGCTCTGCACAACCACTACACGCAGAAGTCTCTCTC |
| | CCTGTCTCCGGGAAAA |
| 30 | TRRMMRTKMRMRRMRRTRRKMRRKMSPARPRTSCREACLQGWTEA |
| 31 | NCRRMMRTKMRMRRMRRTRRKMRRKMSPARPRTSCREACLQGWTEA |
| 32 | QRTRRMMRTKMRMRRMRRTRRKMRRKMSPARPRTSCREACLQGWTEA |
| 33 | RRRQRTRRMMRTKMRMRRMRRTRRKMRRKMSPARPRTSCREACLQGWTEA |
| 34 | GRTRRMMRTKMRMRRMRRTRRKMRRKMSPARPRTSCREACLQGWTEA |
| 35 | RRQRTRRMMRTKMRMRRMRRTRRKMRRKMSPARPRTSCREACLQGWTEA |
| 36 | RRMMRTKMRMRRMRRTRRKMRRKMSPARPRTSCREACLQGWTEA |
| 37 | RRQRTRRMMRTKMRMRRMRRTRRKMRRKMSPARPRTSCREACLQGWTEA |
| 38 | RQRTRRMMRTKMRMRRMRRTRRKMRRKMSPARPRTSCREACLQGWTEA |
| 39 | MCRRMMRTKMRMRRMRRTRRKMRRKMSPARPRTSCREACLQGWTEA |
| 40 | DQRQRTRRMMRTKMRMRRMRRTRRKMRRKMSPARPRTSCREACLQGWTEA |
| 41 | RRRRQRTRRMMRTKMRMRRMRRTRRKMRRKMSPARPRTSCREACLQGWTEA |
| 42 | QRRRQRTRRMMRTKMRMRRMRRTRRKMRRKMSPARPRTSCREACLQGWTEA |
| 43 | RRRQRTRRMMRTKMRMRRMRRTRRKMRRKMSPARPRTSCREACLQGWTEA |
| 44 | RRRERTRRMMRTKMRMRRMRRTRRKMRRKMSPARPRTSCREACLQGWTEA |
| 45 | QRRQRTRRMMRTKMRMRRMRRTRRKMRRKMSPARPRTSCREACLQGWTEA |
| 46 | RRQWTRRMMRTKMRMRRMRRTRRKMRRKMSPARPRTSCREACLQGWTEA |
| 47 | RMMRTKMRMRRMRRTRRKMRRKMSPARPRTSCREACLQGWTEA |
| 48 | RQRTRRMMRTKMRMRRMRRTRRKMRRKMSPARPRTSCREACLQGWTEA |
| 49 | GRRQRTRRMMRTKMRMRRMRRTRRKMRRKMSPARPRTSCREACLQGWTEA |
| 50 | AFKRTRRMMRTKMRMRRMRRTRRKMRRKMSPARPRTSCREACLQGWTEA |
| 51 | NAKRRRRQRTRRMMRTKMRMRRMRRTRRKMRRKMSPARPRTSCREACLQGWTE |
| 52 | CVRRRRQRTRRMMRTKMRMRRMRRTRRKMRRKMSPARPRTSCREACLQGWTEA |
| 53 | RRQRTRRMMRTKMRMRRMRRTRRKMRRKMSPARPRTSCREACLQGWTEA |
| 54 | RQRTRRMMRTKMRMRRMRRTRRKMRRKMSPARPRTSCREACLQGWTEA |
| 55 | NAKRRRRQRTRRMMRTKMRMRRMRRTRRKMRRKMSPARPRTSCREACLQGWTE |
| 56 | CFAKRRRRQRTRRMMRTKMRMRRMRRTRRKMRRKMSPARPRTSCREACLQGWT |
| 57 | RRMMRTKMRMRRMRRTRRKMRRKMSPARPRTSCREACLQGWTEA |
| 58 | PPLCLRRMMRTKMRMRRMRRTRRKMRRKMSPARPRTSCREACLQGWTEA |
| 59 | DHPCRRMMRTKMRMRRMRRTRRKMRRKMSPARPRTSCREACLQGWTEA |
| 60 | GNCRRMMRTKMRMRRMRRTRRKMRRKMSPARPRTSCREACLQGWTEA |
| 61 | CRRMMRTKMRMRRMRRTRRKMRRKMSPARPRTSCREACLQGWTEA |
| 62 | CRRMMRTKMRMRRMRRTRRKMRRKMSPARPRTSCREACLQGWTEA |
| 63 | TCRRMMRTKMRMRRMRRTRRKMRRKMSPARPRTSCREACLQGWTEA |
| 64 | ICRRMMRTKMRMRRMRRTRRKMRRKMSPARPRTSCREACLQGWTEA |
| 65 | CRRMMRTKMRMRRMRRTRRKMRRKMSPARPRTSCREACLQGWTEA |
| 66 | QSVYNNNW |
| 67 | VIGNTGDTYYADSAKG |
| 68 | GIDLSNN |
| 69 | GNTGD |
| 70 | GIDLSNNA |
| 71 | IGNTGDT |
| 72 | VRGPPSYSSSVKNI |
| 73 | RYNMN |
| 74 | SISTSSNYIYYADSVKG |
| 75 | GFTFSRY |
| 76 | STSSNY |
| 78 | GFTFSRYN |
| 79 | ISTSSNYI |
| 80 | TRGWGPFDY |
| 81 | QSIGTA |
| 82 | DIQMTQSPSSLSASVGDRVTITCRARQSIGTAIHWYQQKPGCAPKLLIKYASESISGVPSRFSGSG |
| | SGTDFTLTISSLQPEDFATYYCQQSGSWPYTFGQGTKLEIKGGGSGGSGGCPPCGGSGGEVQLV |
| | ESGGGLVKPGGSLRLSCAASGFTFSRYNMNWVRQAPGKGLEWVSSISTSSNYIYYADSVKGRF |
| | TFSRDNAKNSLDLQMSGLRAEDTAIYYCTRGWGPFDYWGCGTLVTVSS |

1. A method of inhibiting the growth or proliferation of a myeloproliferative neoplasm (MPN) or treating a MPN, the method comprising administering to a subject, preferably a human subject, in need thereof a treatment dose of 0.6-400 mg per administration of an anti-mutant calreticulin (CALRmut)/anti-CD3 bispecific antibody, wherein the anti-CALRmut/anti-CD3 bispecific antibody comprises a first antigen binding domain that binds specifically to CALRmut, and a second antigen binding domain that binds specifically to CD3ε.

2. The method of claim 1, wherein the first antigen binding domain comprises a first HCDR1, a first HCDR2 and a first HCDR3 of a first heavy chain variable region (VH1) of SEQ ID NO:14, and the first antigen binding domain further comprises a first light chain complementarity determining region (LCDR) 1, a first LCDR2, and a first LCDR3 of a first light chain variable region (VL1) of SEQ ID NO:16, and wherein the first HCDR1, first HCDR2, first HCDR3, first LCDR1, first LCDR2 and first LCDR3 are defined by the Kabat, Chothia, IMGT or AbM numbering system.

3. The method of claim 2, wherein the first antigen binding domain comprises the first HCDR1, the first HCDR2, the first HCDR3, the first LCDR1, the first LCDR2 and the first LCDR3 having the amino acid sequence of:
1) SEQ ID NOs:11, 12, 13, 7, 8, and 9, respectively;
2) SEQ ID NOs: 68, 69, 13, 7, 8, and 9, respectively;
3) SEQ ID NOs: 10, 67, 13, 7, 8, and 9, respectively; or
4) SEQ ID NOs: 70, 71, 72 and 66, the amino acid sequence of DAS, and SEQ ID NO: 9, respectively.

4. The method of claim 3, wherein the first antigen binding domain comprises a heavy chain variable region having an amino acid sequence that is at least 90%, 91%, 92%, 93%, 94%, 95%, 96%, 97%, 98%, or 99% identical to the amino acid sequence of SEQ ID NO:14, and a light chain variable region having an amino acid sequence that is at least 90%, 91%, 92%, 93%, 94%, 95%, 96%, 97%, 98%, or 99% identical to the amino acid sequence of SEQ ID NO:16.

5. The method of claim 1, wherein the first antigen binding domain comprises the VH1 of SEQ ID NO:14, and the VL1 of SEQ ID NO:16.

6. The method of claim 1, wherein the first antigen binding domain comprises a Fab.

7. The method of claim 1, wherein the second antigen binding domain comprises a second HCDR1, a second HCDR2, and a second HCDR3 of a second heavy chain variable region (VH2) of SEQ ID NO:23 and a second LCDR1, a second LCDR2, and a second LCDR3 of second light chain variable region (VL2) of SEQ ID NO:27, wherein the second HCDR1, second HCDR2, second HCDR3, second LCDR1, second LCDR2 and second LCDR3 are defined by the Kabat, Chothia, IMGT or AbM numbering system.

8. The method of claim 7, wherein the second antigen binding domain comprises the second HCDR1, the second HCDR2, the second HCDR3, the second LCDR1, the second LCDR2 and the second LCDR3 having the amino acid sequence of:
1) SEQ ID NOs:20, 21, 22, 24, 25, and 26, respectively;
2) SEQ ID NOs: 75, 76, 22, 24, 25, and 26, respectively;
3) SEQ ID NOs: 73, 74, 22, 24, 25, and 26, respectively; or
4) SEQ ID NOs: 78, 79, 80 and 81, the amino acid sequence of YAS, and SEQ ID NO: 26, respectively.

9. The method of claim 8, wherein the second antigen binding domain comprises a heavy chain variable region having an amino acid sequence that is at least 90%, 91%, 92%, 93%, 94%, 95%, 96%, 97%, 98%, or 99% identical to the amino acid sequence of SEQ ID NO:23, and a light chain variable region having an amino acid sequence that is at least 90%, 91%, 92%, 93%, 94%, 95%, 96%, 97%, 98%, or 99% identical to the amino acid sequence of SEQ ID NO:27.

10. The method of claim 9, wherein the second antigen binding domain comprises the VH2 of SEQ ID NO:23, and the VL2 of SEQ ID NO:27.

11. The method of claim 1, wherein the second antigen binding domain comprises an scFv.

12. The method of claim 11, wherein the scFv is a spFv having an amino acid sequence that is at least 90%, 91%, 92%, 93%, 94%, 95%, 96%, 97%, 98%, or 99% identical to the amino acid sequence of SEQ ID NO:82.

13. The method of claim 1, wherein the first binding domain comprises a Fab comprising a VH1 having the amino acid sequence of SEQ ID NO:14 and a VL1 having the amino acid sequence of SEQ ID NO:16; and wherein the second binding domain comprises an spFv having the amino acid sequence of SEQ ID NO:82.

14. The method of claim 1, wherein the first antigen binding domain is linked to a first immunoglobulin (Ig) constant region or a fragment of the first Ig constant region and/or the second antigen binding domain is linked to a second immunoglobulin (Ig) constant region or a fragment of the second Ig constant region, wherein the first Ig constant region or the fragment of the first Ig constant region and/or the second Ig constant region or the fragment of the second Ig constant region is of an IgG1, an IgG2, and IgG3 or an IgG4 isotype.

15. The method of claim 14, wherein the first Ig constant region or the fragment of the first Ig constant region and/or the second Ig constant region or the fragment of the second Ig constant region is of an IgG1 isotype.

16. The method of claim 14, wherein the first Ig constant region or the fragment of the first Ig constant region and/or the second Ig constant region or the fragment of the second Ig constant region comprises at least one mutation that results in reduced binding of the anti-CALRmut/anti-CD3 bispecific antibody to a Fc R, such as one, two or three of the mutations of L234A, L235A and D265S, wherein the residues are numbered according to the EU index of Kabat.

17. The method of claim 16, wherein the Fc R is Fc RI, Fc RIIA, Fc RIIB or Fc RIII, or any combination thereof.

18. The method of claim 14, wherein the first Ig constant region or the fragment of the first Ig constant region and the second Ig constant region or the fragment of the second Ig constant region comprise one or more heterodimerization mutations to promote heterodimerization, such as knob-into-hole (KiH) substitutions.

19. The method of claim 18, wherein one of the first Ig constant region (or the fragment of the first Ig constant region) and the second Ig constant region (or the fragment of the second Ig constant region) comprises the heterodimerization mutation T366W, and the other one of the first Ig constant region (or the fragment of the first Ig constant region) and the second Ig constant region (or the fragment of the second Ig constant region) comprises the heterodimerization mutations T366S, L368A and Y407V.

20. The method of claim 14, wherein one of the first Ig constant region (or the fragment of the first Ig constant region) and the second Ig constant region (or the fragment of the second Ig constant region) further comprises the mutations H435R and Y436F.

21. The method of claim 1, wherein the anti-CALRmut/anti-CD3 bispecific antibody comprises a first heavy chain (HC1) having an amino acid sequence that is at least 90%, 91%, 92%, 93%, 94%, 95%, 96%, 97%, 98%, or 99% identical to the amino acid sequence of SEQ ID NO:15 and a first light chain (LC1) having an amino acid sequence that is at least 90%, 91%, 92%, 93%, 94%, 95%, 96%, 97%, 98%, or 99% identical to the amino acid sequence of SEQ ID NO:17, and a second heavy chain (HC2) having an amino acid sequence that is at least 90%, 91%, 92%, 93%, 94%, 95%, 96%, 97%, 98%, or 99% identical, to the amino acid sequence of SEQ ID NO: 28.

22. The method of claim 21, wherein the anti-CALRmut/anti-CD3 bispecific antibody comprises the HC1 having the amino acid sequence of SEQ ID NO:15, the LC1 having the amino acid sequence of SEQ ID NO:17 and the HC2 having the amino acid sequence of SEQ ID NO:28.

23. The method of claim 1, wherein the treatment dose of the anti-CALRmut/anti-CD3 bispecific antibody is administered subcutaneously, preferably at the treatment dose of 20-200 mg per administration.

24. The method of claim 1, wherein the anti-CALRmut/anti-CD3 bispecific antibody is administered once every 1 to 8 weeks.

25. The method of claim 24, wherein the treatment dose of the anti-CALRmut/anti-CD3 bispecific antibody is administered once every three weeks.

26. The method of claim 24, wherein the treatment dose of the anti-CALRmut/anti-CD3 bispecific antibody is administered once every week.

27. The method of claim 24, wherein the treatment dose of the anti-CALRmut/anti-CD3 bispecific antibody is administered once every two weeks.

28. The method of claim 24, wherein the treatment dose of the anti-CALRmut/anti-CD3 bispecific antibody is administered once every four weeks.

29. The method of claim 1, further comprising administering to the subject one or more step-up doses of the anti-CALRmut/anti-CD3 bispecific antibody prior to the administration of the treatment dose, wherein none of the step-up doses exceed the treatment dose.

30. The method of claim 29, wherein the step-up dose is administered to the subject 3-8 days, such as 3, 4, 5, 6, 7, or 8 days, prior to the initial administration of the treatment dose.

31. The method of claim 29, comprising administering to the subject a first step-up dose and a second step-up dose of the anti-CALRmut/anti-CD3 bispecific antibody, and wherein the first step-up dose is administered prior to the administration of the second step-up dose, preferably 3-8 days, such as 3, 4, 5, 6, 7, or 8 days, prior to the administration of the second step-up dose, and wherein the second step-up dose is administered prior to the administration of the initial treatment dose, preferably 3-8 days, such as 3, 4, 5, 6, 7, or 8 days, prior to the administration of the initial treatment dose.

32. The method of claim 31, wherein the first step-up dose does not exceed the second step-up dose, and the second step-up dose does not exceed the treatment dose initially administered.

33. A method of treating a myeloproliferative neoplasm, the method comprising subcutaneously administering to a human subject in need thereof, once every three weeks, a treatment dose of 1.2-400 mg per administration, such as 20-200 mg per administration, of an anti-CALRmut/anti-CD3 bispecific antibody, wherein the anti-CALRmut/anti-CD3 bispecific antibody comprises a first heavy chain having the amino acid sequence of SEQ ID NO:15, a first light chain having the amino acid sequence of SEQ ID NO:17 and a second heavy chain having the amino acid sequence of SEQ ID NO:28, optionally, the method further comprises subcutaneously administering to the subject a step-up dose of 0.6 or 1.2 mg per administration of the anti-CALRmut/anti-CD3 bispecific antibody one week before the initial administration of the treatment dose.

34. The method of claim 33, wherein the MPN is characterized by the presence of a mutant calreticulin, such as Type 1 like and/or Type 2 like CALR mutations.

35. The method of claim 33, wherein the subject is ineligible, intolerant or resistant to JAK inhibitor therapy.

36. The method of claim 33, wherein the subject has a splenectomy.

37. The method of claim 33, wherein the subject has an allograft, e.g., an allogeneic bone marrow or stem cells transplant.

38. The method of claim 33, wherein the subject has been administered a prior therapy for treating the MPN, such as a JAK inhibitor and/or hydroxyurea, optionally, the subject has failed one or more lines of prior treatments.

39. The method of claim 33, wherein the MPN is selected from the group consisting of chronic myelogenous leukemia, polycythemia vera, primary myelofibrosis (MF), essential thrombocythemia (ET), chronic neutrophilic leukemia, and chronic eosinophilic leukemia, such as a myelostratic syndrome selected from ET, prefibrotic MF, overt primary MF, and accelerated blast phase ME.

40. The method of claim 39, wherein the subject is diagnosed with ET, particularly an ET with high-risk of thrombosis or hemorrhage, defined as any 1 of the following:
age >60 years,
platelet count >1500×109/L at any point during the participant's disease,
previous documented thrombosis (including transient ischemic attack [TIA]), erythromelalgia, or migraine (severe, recurrent, requiring medications, and felt to be secondary to the MPN) either after diagnosis or within 10 years before diagnosis and considered to be disease-related,
previous hemorrhage or coagulopathy related to ET, or
diabetes mellitus or hypertension requiring pharmacological therapy >6 months and
intolerant or resistant or refractory to hydroxyurea (HU), defined as any 1 of the following according to NCCN Guidelines Version 1.2023:
platelet count >600×109/L after 3 months of at least 2 g/day or maximum tolerated dose (MTD) of HU (2.5 g/day in participants with a body weight >80 kg),
platelet count >400×109/L and WBC <2.5×109/L at any dose of HU (for a period of at least 3 months),
platelet count >400×109/L and hemoglobulin <10 g/dL at any dose of HU (for a period of at least 3 months),
presence of leg ulcers or other unacceptable mucocutaneous manifestations at any dose of HU, or
HU-related fever.

41. The method of claim 40, wherein the method results in at least one of: 1) normal spleen size on imaging; and 2) platelet count ≤400×109/L and/or white cell count ≤10×109/L in peripheral blood.

42. The method of claim 39, wherein the subject is diagnosed with MF, particularly a primary MF, such as a primary MF with a Dynamic International Prognostic Scoring System (DIPSS) risk score of Intermediate 1 (Int-1), Intermediate 2 (Int-2) or High-Risk (HR), optionally with a blast percentage not consistently exceeding 20% in blood or bone marrow; or a post-ET MF, such as a post-ET MF with a Myelofibrosis Secondary to PV and ET-Prognostic Model (MYSEC-PM) risk score of Int-1, Int-2 or HR, optionally with a blast percentage not consistently exceeding 20% in blood or bone marrow.

43. The method of claim 42, wherein the method results in a reduction in splenic volume of the subject compared to a baseline splenic volume measured before administration of the anti-mutant calreticulin (CALRmut)/anti-CD3 bispecific antibody, preferably the splenic volume is reduced by at least 35% as compared to a baseline splenic volume measured before administration of the anti-mutant calreticulin (CALRmut)/anti-CD3 bispecific antibody.

44. The method of claim 42, wherein the method results in at least one of: 1) bone marrow age-adjusted normocellularity, 2) <5% blasts, and 3) Hemoglobin ≥10 g/dL, neutrophil count ≥1×109/L, and/or platelet count ≥100×109/L in peripheral blood of the subject.

45. The method of claim 33, wherein the method decreases soluble CALRmut levels in serum of the subject, preferably the soluble CALRmut levels are reduced by at least 50% compared to a baseline level measured before administration of the anti-mutant calreticulin (CALRmut)/anti-CD3 bispecific antibody.

46. The method of claim 33, wherein the method results in a reduction of CALRmut positive cells in bone marrow of the subject.

47. The method of claim 33, wherein the method results in improved bone marrow architecture, such as reduction in bone marrow reticulin fibrosis and bone marrow cellularity.

48. The method of claim 33, further comprising administering to the subject at least one first additional therapeutic prior to being administered with the treatment dose of the anti-CALRmut/anti-CD3 bispecific antibody, or prior to being administered with the set-up dose of the anti-CALRmut/anti-CD3 bispecific antibody.

49. The method of claim 48, wherein the at least one first additional therapeutic is a glucocorticosteroid, antihistamine, antipyretic, antiemetic, or any combination thereof.

50. The method of claim 48, wherein at least one first additional therapeutic is dexamethasone.

51. The method of claim 48, wherein at least one first additional therapeutic is diphenhydramine or equivalent.

52. The method of claim 48, wherein at least one first additional therapeutic is acetaminophen.

53. The method of claim 48, wherein the at least one first additional therapeutic is ondansetron or equivalent thereof.

54. The method of claim 1, further comprising administering to the subject at least one second additional therapeutic prior to, and optionally after, being administered with the treatment dose of the anti-CALRmut/anti-CD3 bispecific antibody.

55. The method of claim 54, wherein the at least one second additional therapeutic is a H1 antagonist, H2 antagonist or a Leukotriene inhibitor, or any combination thereof.

56. The method of claim 54, wherein the at least one second additional therapeutic is loratadine, cetirizine or equivalent thereof.

57. The method of claim 54, wherein the at least one second additional therapeutic is ranitidine, or equivalent thereof.

58. The method of claim 54, wherein the at least one second additional therapeutic is montelukast, or equivalent thereof.

59. An anti-CALRmut/anti-CD3 bispecific antibody for use in the method of claim 33.
